(19) United States
(12) Patent Application Publication
(10) Pub. No.: US 2008/0242834 A1
(43) Pub. Date: Oct. 2, 2008

(54) Methods of Predicting Hyp-Glycosylation Sites For Proteins Expressed and Secreted in Plant Cells, and Related Methods and Products

(75) Inventors: Marcia J. Kieliszewski, Albany, OH (US); Jianfeng Xu, Athens, OH (US)

Correspondence Address: WOOD, HERRON & EVANS, LLP, 2700 CAREW TOWER, 441 VINE STREET, CINCINNATI, OH, 45202, US

(73) Assignee: OHIO UNIVERSITY, Athens, OH (US)

(21) Appl. No.: 11/995,063

(22) PCT Filed: Jul. 10, 2006
(86) PCT No.: PCT/US2006/026594

§ 371 Date: Mar. 14, 2008

Related U.S. Application Data
(60) Provisional application No. 60/697,337, filed on Jul. 8, 2005.

(51) Int. Cl. C07K 2/00; C12P 21/00

(52) U.S. Cl. 530/300; 435/69.1

(57) ABSTRACT

Proteins with Hyp-glycosylation are more likely to be secreted in plant cells at high levels than those without. Methods are disclosed for the prediction of Pro-hydroxylation and Hyp-glycosylationsites in proteins. Such methods can be used to identify (1) proteins which, without modification, are predisposed to develop Hyp-glycosylation, if expressed in plant cells, and (2) modifications (especially substitution mutations) which increase the propensity of a protein to develop Hyp-glycosylation, with a view to high level or increased secretion. It is also possible to determine empirically whether a particular protein will undergo Hyp-glycosylation suitable for the desired level of secretion in plant cells. Both modified proteins, and methods for the expression and secretion of predisposed and modified proteins, are claimed.

This application claims the benefit, under 35 USC 119(e), of prior U.S. provisional application 60/697,337, filed Jul. 8, 2005, and incorporated by reference in its entirety.

### CROSS-REFERENCE TO RELATED APPLICATIONS

The instant application is related most closely to the following prior applications: U.S. Provisional Appls. 60/536,486, filed Jan. 14, 2004; 60/582,027, filed Jun. 22, 2004; and 60/602,562, filed Aug. 18, 2004, and PCT/US2005/001160 and U.S. Ser. No. 11/036,256, both filed Jan. 14, 2005, all of which are hereby incorporated by reference in their entirety.

### MENTION OF GOVERNMENT RIGHTS

The work leading to this invention was supported, at least in part, by NSF Grant No. MCB9874744 and USDA Project No. OHOW200206201. The U.S. government has certain rights in the invention.

### BACKGROUND OF THE INVENTION

1. Field of the Invention

This invention relates to the secretion of proteins in plant cells.

2. Description of the Background Art

In 1966, Edwin H. Eylar proposed that all glycosylation, regardless of amino acid addition site, enhances secretion. Eylar, “On the biological role of glycoproteins,” Journal of Theoretical Biology, Vol 10, issue 1, pp 89-113 (1966). However, his hypothesis was dismissed by the scientific community after the discovery of signal peptide sequences, which were credited as the sole agent needed for protein secretion. See P J Winterburn and C. F. Phelps (1972) The significance of glycosylated proteins, Nature Vol 235, Mar. 24, 1972. Winterbourn concludes, “there is no substance in the belief that carbohydrates are added as passports for export from the cell.” Instead, Winterbourn suggested that “sugars are included in protein structures as a means of coding for the topographical location within the organism.”

Spiro, “protein glycosylation: nature, distribution, enzymatic formation, and disease implications of glycopeptide bonds,” Glybiology, 12(4): 43R-56R (2002) presents a mini-review of the subject. According to Spiro, O-glycosylation occurs at Ser, Thr, Tyr, Hyp (hydroxyproline) and Hyl (hydroxylysine) residues, and N-glycosylation at Asn and Arg. Spiro notes that Gal and Ara saccharides linked to Hyp are features of plant glycoproteins, and states that for arabinosylation of Hyp, the consensus site is a repetitive Hyp rich domain, e.g., Lys-Pro-Hyp-Hyp-Val, SEQ ID NO:1).

Support of young growing plant tissues depends largely on the turgidity of cells restrained by an elastic cell wall comprised of three interpenetrating networks, namely, cellulosic-xyloglucan, pectin, and hydroxyproline-rich glycoproteins (HRGPs). When these networks are loosened, turgor drives cell extension. Significantly, HRGPs have no animal homologs, thus emphasizing a plant-specific function.

Quantitatively, most of the cell surface HRGPs (extensins) form a covalently cross-linked cell wall network. Unlike extensins, another set of HRGPs, arabinogalactan-proteins (AGPs) occur as monomers that are hyperglycosylated by arabinogalactan polysaccharides. AGPs are initially tethered to the plasma membrane by a lipid anchor whose cleavage results in their movement from the periplasm through the cell wall to the exterior. Although implicated in diverse aspects of plant growth and development, the precise functions of AGPs remain unclear.

Shpak, Leykam, and Kieliszewski, “Synthetic genes for glycoprotein design and the elucidation of hydroxyproline-O-glycosylation codes”, Proc. Nat. Acad. Sci. (USA), 96(26: 14736-14741 (Dec. 21, 1999), explains that hydroxyproline (Hyp)-O-glycosylation uniquely characterizes an ancient and diverse group of structural glycoproteins associated with the cell wall. These Hyp-rich glycoproteins (HRGPs) are broadly implicated in all aspects of plant growth and development, including fertilization, differentiation and tissue organization, control of cell expansion growth, and responses to stress and pathogenesis.

There are three major HRGP families: arabinogalactan proteins (AGPs), extensins, and proline-rich proteins (PRPs). AGPs [>90% (wt/wt) sugar] have repetitive variants of (Xaa-Hyp)n motifs with O-linked arabinogalactan polysaccharides involving an O-galactosyl-Hyp glycosidic bond. Extensins [50% (wt/wt) sugar] have a diagnostic Ser-Hyp4 repeat that contains short oligosaccharides of arabinose (Hyp arabinosides) involving an O-L-arabinosyl-Hyp linkage. Finally, the lightly arabinosylated PRPs [2-27% (wt/wt) sugar] are the most highly periodic, consisting largely of pentapeptide repeats, typically variants of Pro-Hyp-Val-Tyr-Lys (SEQ ID NO:2). Recombinant production of some Hyp-rich glycoproteins is discussed in Kielizewski et al., U.S. Pat. Nos. 6,548,642, 6,570,062, and 6,639,050.

According to the Hyp contiguity hypothesis, discussed in Shpak et al. (1999) but advanced previously, clustered, noncontiguous Hyp residues (e.g., Hyp's in Xaa-Hyp-Xaa-Hyp) are sites of arabinogalactan polysaccharide attachment, while small arabinooligosaccharides (1-5 Ara residues/Hyp) are attached to contiguous (dipeptidyl or larger) Hyp residues. Di-Hyp blocks are found in PRPs and tetra-Hyp blocks in extensins.

Shpak et al. (1999) expressed two synthetic genes, encoding putative AGP glycomodules, in plants. “The construct expressing noncontiguous Hyp [32 Ser-Hyp repeats] showed exclusive polysaccharide addition, whereas another construct containing noncontiguous Hyp and additional contiguous Hyp [contained three repeats of a 19 amino acid sequence, SOOOTLSOSOTOTOOOGPH, SEQ ID NO: 3, from gum arabic glycoprotein, GAGP] showed both polysaccharide and arabinooligosaccharide addition consistent with the predictions of the Hyp contiguity hypothesis.”

Shpak, et al., “Contiguous hydroxyproline residues direct hydroxyproline arabinosylation in Nicotiana tabacum”, J. Biol. Chem. 276(14): 11272-8 (2001) sought to determine the minimum level of Hyp contiguity to achieve arabinosylation by expressing synthetic genes encoding repetitive (Ser-Pro-Pro), (Ser-Pro-Pro-Pro, SEQ ID NO:4), and (Ser-Pro-Pro-Pro-Pro, SEQ ID NO:5). Half of the Hyp residues in the di-Hyp blocks were arabinosylated, and almost 100% of those in the tetra-Hyp blocks. In the case of the tri-Pro blocks, these were incompletely hydroxylated at each of the three Pro's, resulting in a mixture of contiguous and non-contiguous Hyp and thus in partial arabinosylation.

Schultz C J, Rumsewicz M R, Johnson K L, Jones B J Gaspar Y and Bacic A (2002). Using genomic resources to guide research directions: The arabinogalactan-protein gene family as a test case. Plant Physiol. 129, 1448-1463. describes a computer program to look for AGPs.

The first criterion for classification as an AGP was that the protein had a PAST (Pro, Ala, Ser, Thr content) over 50%. The second criterion was that the protein had an N-terminal signal sequence identifiable by the program SignalP, see Nielsen et al., Protein Eng 10:1-6 (1997). Applied to the known proteins encoded by the Arabidopsis genome, 62 proteins were identified by the first criterion, of which 49 were predicted to be secreted. Schultz et al. admit that the 50% PAST threshold did not pickup PRP1-PRP4, for which the PAST value is 32-45%.

Schultz et al. also identified putative AG peptides by the following criteria: length of 50-75 amino acids; PAST composition of over 35%; and predicted to be secreted.

FLAs could not be found by a simple biased amino acid composition search because they are chimeric AGPs, that is, they include fasciclin domains, which are not AGP-like glycomodule domains. For example, the FLA7 protein is 39% PAST, but if the fasciclin domain is ignored, it is 52% PAST. Schultz therefore screened for Arabidopsis proteins which were at least 39% PAST. Schultz et al. then used a hidden markov model for 88 known fasciclin domains to create a position-specific score matrix for identification of fasciclin domains.

Schultz et al. suggest that additional proteins containing AGP glycomodules might be found by calculating the PAST percentage in overlapping windows of 15-25 amino acid residues.

Shimizu, et al., “Experimental determination of proline hydroxylation and hydroxyproline arabinogalactosylation motifs in secretory proteins,” Plant Journal (2005) (doi: 10.1111/j.1365-313X.2005.02419.x) postulates both proline hydroxylation and hydroxyproline arabinogalactosylation motifs. These were identified by studying deletion and substitution mutants of plant sporamins.

According to Shimizu et al., hydroxylation of a proline residue requires the five amino acid sequence

[AVSTG]-Pro-[AVSTGA]-[GAVPSTC]-[APS or acidic]

(where Pro is the modification site)

Glycosylation of hydroxyproline (Hyp), according to Shimizu et al., requires the seven amino acid sequence

[not basic]-[not T]-[neither P, T, nor amide]-Hyp-[neither amide nor P]-[not amide]-[APST], although charged amino acids at the −2 position and basic amide residues at the +1 position relative to the modification site seem to inhibit the elongation of the arabinogalactan side chain.

Based on the combination of these two requirements, Shimizu et al. concluded that the sequence motif for efficient hydroxylation followed by arabinogalactosylation, including the elongation of the glycan side chain, is

[not basic]-[not T]-[AVSG]-Pro-[AVST]-[GAVPSTC]-[APS].

Shimizu does not propose mutating any non-plant protein so that it can be secreted, or secreted more efficiently, in plant cells. Shimizu does not propose expressing, in secretible form, any plant protein which is not natively secreted, even if that protein natively has the postulated Hyp-glycosylation motif. Shimizu does not propose mutating any plant protein which does not include any sequences fitting the motif so that it possesses the motif. Shimizu does not propose mutating any plant protein to increase the number of prolines which fit the motif.

Russell, U.S. Pat. No. 6,080,560, “Method for producing antibodies in plant cells”, reports that the chimeric L6 single chain antibody was expressed and secreted at high levels in tobacco NT1 cells. The expression system included a gene encoding a tobacco 5′ extensin or cotton signal sequence, and an sFv antigen recognition sequence, under the transcriptional control of a CaMV 35S promoter and an nos poly A addition sequence. The reported yields were as high as 200 mg/L.

Russell did not deliberately mutate the sFv-encoding sequence in order to facilitate expression and secretion in plant cells, and did not state any opinion as to why the single chain antibody was so efficiently produced therein. However, the present inventors believe that Russell unsuspectingly chose to produce a single chain antibody which had several prolines which, according to the predictions of the present inventor's algorithm, would be hydroxylated and O-glycosylated, thus resulting in high-level secretion. That algorithm predicts that six of the prolines in Russell SEQ ID NO:6 would be so processed. (The present inventors also believe that the Asn-Pro-Ser site in Russell SEQ ID NO:8 would be N-glycosylated.)

Several papers have reported high expression and secretion of proteins which, according to our algorithm, would contain one or more Hyp-glycosylation sites. See Ziegler, et al, “Accumulation of a Thermostable Endo-1,4-beta-D-glucanase in the apoplast of Arabidopsis thaliana leaves,” Molecular Breeding 6:37-46 (2000) (this protein accumulated to a level accounting for 26 of total soluble protein; the glucanase converts cellulose to fermentable glucose); Shin, et al, “High level of expression of recombinant human granulocyte-macrophage colony stimulating factor in transgenic rice cell suspension culture, Biotechnology and Bioengineering, 82(7): 778-83 (2003) (yield of 129 mg·L culture medium. However, none of these authors recognize the relationship between Hyp-glycosylation and high-level expression and secretion in plants.

Gil, et al., “High yield expression of a viral peptide vaccine in transgenic plants,” FEBS Lett., 488: 13-17 (2001) reports expression of a viral peptide vaccine in plants. However, his nucleic acid construct did not include a signal sequence, consequently, the encoded peptide could not have been secreted. Since it was not secreted, the prolines in that sequence could not have been hydroxylated and subsequently glycosylated, as those processes occur in the membrane. The sequence of this viral peptide corresponds to residues 1 to 23 of “virus protein 2”, sequence EMBL database # AAV36761.1, with the position 23 Ser (S) being identified as Glp (Pyrrolidone carboxylic acid (pyroglutamate)) in Gil.

Karnoup, et al., “O-linked glycosylation in maize-expressed human IgA1”, Glycobiology 15(10): 965-81 (published online May 18, 2005) reports that prolines in the conserved heavy chain hinge region, which is rich in proline, experienced hydroxylation and O-linked arabinosylation. The article characterized this, inaccurately, as the first observation of Hyp-glycosylation in a recombinant therapeutic protein in transgenic plants (compare, e.g., PCT/US2005/001160 cited above). In any event, no suggestion was made that Hyp-glycosylation could enhance secretion, etc.

### SUMMARY OF THE INVENTION

This invention arises from the discovery of, first, the “code” controlling whether plant cells hydroxylate proline and glycosylate hydroxyproline in native proteins, and second, the relationship between Hyp-glycosylation and high-level secretion. By exploiting this information, it is possible to recombinantly produce, in plant cells, proteins which are not natively secreted in such cells, and have them secreted at high levels. The plant cells may be in cell culture, in tissue culture, or part of a plant.

When a protein is expressed in a plant, certain prolines may become hydroxylated, and certain of the resulting hydroxyprolines are glycosylated. It is the presence of glycosylated hydroxyprolines which is the most important determinant of the degree of secretion of the protein. Hence, we have developed methods of predicting which prolines will be hydroxylated and which hydroxyprolines will be glycosylated. If these methods are applied to a protein, the glycosylated residues (more specifically, prolines which will be post-translationally modified into arabinosylated or arabinogalactosylated hydroxyproline residues), can be identified in advance. In that manner, we can determine which proteins are likely to be readily secreted if expressed, in secretable form, in plant cells.

One class of proteins of interest are naturally occurring non-plant proteins which fortuitously possess one or more prolines which, if expressed and secreted by suitable plant cells, will be hydroxylated and glycosylated.

Another class of proteins of interest are non-plant proteins which are deficient in favorable prolines, but which can be engineered, based on the design methods set forth in this disclosure, to remedy this deficiency.

A third class of proteins of interest are plant proteins which are not naturally secreted, but which, if expressed as fusion proteins including a suitable signal peptide, fortuitously possess the favorable prolines.

A fourth class of proteins of interest are plant proteins which are deficient in favorable prolines, but which can be engineered to remedy this deficiency.

It will be appreciated that, among non-plant proteins, human proteins, or mutants thereof, are of particular interest. The discussion of human proteins which follows applies, mutatis mutandis, to other proteins of interest.

Thus, if the goal is to use plant cell culture to produce a protein having the biological activity of a human protein of interest, the first step is to analyze the sequence of the human protein and determine whether it would, without modification, be hydroxylated and glycosylated by plant cells in such a manner as to achieve the desired level of secretion. If so, then this invention teaches that it is desirable that a mature protein coding sequence, suitable for plant cell expression, and operably linked to a signal sequence functional in plant cells, and to a promoter functional in plant cells, be introduced into such cells, and the transformed plant cells cultivated under conditions in which that human protein is expressed and secreted.

If the sequence of the human protein is not such as would achieve a desired level of secretion, then one may instead produce a mutant protein which does achieve that level, and which either retains substantially all of the desired biological activity of the reference human protein, or which can be processed (e.g., cleaved), in the culture medium or at a later stage of recovery, to yield a final protein which does satisfy this biological activity test.

There are two major approaches to designing a suitable mutant protein. In the first approach (described in our prior related applications cited above, but further refined here), the human protein is mutated by insertion of at least one “Hyp-glycomodule” at the amino and/or carboxy ends of the protein (in which case the reader may prefer to speak of the glycomodule as being “added” to the protein). The term “Hyp-glycomodule” refers generally to a sequence containing one or more prolines so positioned that the plant cell will hydroxylate and glycosylate them (hence the “glyco” of the name). The term will be defined more precisely in a later section of this application.

It is quite common for proteins with biological activity to have at least one free end, to which additional amino acids can be attached without substantial loss of biological activity. The glycomodule addition strategy exploits this aspect of protein behavior.

Moreover, it is possible to link the Hyp-glycomodule to the native human protein moiety by a spacer which either 1) acts to distance the native human protein moiety from the Hyp-glycomodule in such manner as to increase the retention of native human protein biological activity by the Hyp-glycomodule-spacer-human protein fusion relative to that retained by a direct Hyp-glycomodule-human protein fusion, or 2) provides a site-specific cleavage site for an enzyme or chemical agent such that, after cleavage at that site, a new product is generated which does have the desired biological activity.

In addition to, or instead of, using a spacer, it is possible that if the addition of the Hyp-glycomodule results in reduction of biological activity, that this can be ameliorated by mutations within the human protein moiety proper. These mutations may be substitution mutations (not necessarily introducing prolines) or truncation of one or more amino acids from either or both ends of the human protein (e.g., so that the Hyp-glycomodule is in whole or in part replacing an amino or carboxy sequence).

In the second strategy, the human protein is mutated internally. Most often, this will be by one or more substitution mutations which introduce prolines at sites collectively favored for hydroxylation and subsequent glycosylation. Alternatively or additionally, amino acids in the vicinity of a native or introduced proline may be replaced with other amino acids, so that said native or introduced proline becomes one collectively favored for hydroxylation and subsequent glycosylation. Of course, any other desired substitutions can be made if they do not substantially adversely affect either plant cell secretion or (with certain caveats) the biological activity of the mutant protein. It is also possible, although more difficult from the standpoint of preserving biological activity, to foster proline hydroxylation and subsequent hydroxyproline glycosylation by deletion and/or internal insertion.

It should be recognized that the first strategy in effect creates a Hyp-glycomodule within the protein by addition, whereas the second does so by substitution and/or deletion and/or internal insertion.

These two approaches may of course be combined, that is, one can attach a Hyp-glycomodule to one end of a human protein and also introduce glycosylation-increasing substitution mutations into the human protein moiety.

In any event, proteins comprising at least one native Hyp-glycomodule and/or at least one substitution and/or at least one internal insertion Hyp-glycomodule, whether or not they also comprise an addition Hyp-glycomodule, are of particular interest. However, proteins comprises only one or more addition Hyp-glycomodules and no substitution Hyp-glycomodules are also within the contemplation of the present invention.

It is worth noting that in some instances, the modification may usefully inhibit one of the biological activities of the parental protein, while leaving another biological activity intact. For example, an agonist must bind to and activate a receptor. If the modification inhibits activation, but permits binding, then the agonist is converted into an antagonist. An example of the use of a modification to introduce Hyp-glycosylation while converting an agonist into an antagonist is given in the Examples, in the discussion of Fibroblast Growth Factor 7.

### DETAILED DESCRIPTION OF THE PREFERRED EMBODIMENTS OF THE INVENTION

### Overview

The present invention thus relates, in part, to
methods of predicting Hyp-glycosylation sites in proteins
methods of designing a mutant protein with an increased number of predicted Hyp-glycosylation sites relative to its parental protein
methods of expressing and secreting proteins (including both mutant proteins, and wild-type proteins not previously produced in plant cells), with one or more Hyp-glycosylation sites, in plant cells, where such proteins have not previously been expressed in and secreted by plant cells
non-naturally occurring mutant proteins, with one or more Hyp-glycosylation sites, not previously expressed in and secreted by plant cells, in secreted (mature) form
precursor proteins consisting essentially of a plant specific signal peptide and a mature protein as described above, with one or more Hyp-glycosylation sites, not previously expressed in and secreted by plant cells
DNA sequences encoding such proteins
expression vectors for expressing such mature or precursor proteins in plant cells.

The glycoproteins of the present invention are expected to be more efficiently secreted in plant cells; this of course presumes that they are expressed in a precursor form comprising a secretory signal peptide recognized by the host plant cell, which signal peptide is cleaved off, releasing the mature core protein. Glycosylation is post-translational, and occurs after the signal peptide is removed. In the glycoproteins of the present invention, one or more of the glycosylated residues are hydroxyprolines. Hydroxyprolines arise through hydroxylation of proline residues; it is not presently known whether hydroxylation is co-translational or post-translational, and thus its timing relative to signal peptide cleavage.

The contemplated glycoproteins may exhibit various additional advantages over their wild-type counterparts, including increased solubility, increased resistance to proteolytic enzymes, and/or increased stability. They may have comparable biological activity, or they may have improved pharmacodynamic or pharmacokinetic properties, such as increased biological half-life as compared to wild-type proteins. Finally, glycosylation makes possible the purification of the protein by carbohydrate affinity chromatography.

### DEFINITIONS

A glycoprotein is a protein containing one or more carbohydrate chains. The core of a glycoprotein is the corresponding unglycosylated protein having the same amino acid sequence. This core protein may include non-genetically encoded, and even non-naturally occurring, amino acids.

The sequence as determined solely by the genetic code is referred to as the “genetically encoded sequence”, the “genetically encodable sequence”, the “translated sequence”, the “nascent sequence”, the “initial sequence”, or the “initial core sequence”. In this sequence, what the plant cell might ultimately process into a hydroxyproline, glycosylated or not, is considered merely a proline. The term “proline skeleton” typically refers to this level of sequence analysis.

The sequence resulting from the complete action of the proline hydroxylases of the host cell, but otherwise unprocessed (i.e., no signal peptide cleavage or glycosylation), is referred to as the “core sequence,”, the “modified core sequence”, the “hydroxylase-processed sequence”, or the “intermediate sequence.” It is not in fact known whether the proline hydroxylase action is co-translational, post-translational, or a combination of the two. However, unless otherwise explicitly indicated, the terms in question refer to the sequence in which all prolines which are hydroxylated prior to secretion of the protein are listed as hydroxyprolines, regardless of whether such hydroxylation in fact occurs prior to signal peptidase cleavage. In this sequence, prolines and hydroxyprolines are distinguished, but the state of glycosylation is ignored. The term “hydroxyproline skeleton” refers to this level of sequence analysis.

The portion of the intermediate sequence which ultimately becomes part of the mature protein—that is, which excludes the signal peptide—is referred to as the mature portion.

The “completely processed sequence”, also known as the “mature sequence”, the “secreted sequence” or the “final sequence”, is the result the hydroxylation of the prolines, the removal of the signal peptide, and the glycosylation. In this sequence, prolines, unglycosylated hydroxyprolines, and glycosylated hydroxyprolines are distinguished. However, unless otherwise explicitly indicated, sequences are not distinguished on the basis of the precise nature of the glycosylation at a particular amino acid position. We can however refer to proteins with different “glycosylation patterns.”

The term “predicted Pro-hydroxylation site” means a proline residue which, according to the specified prediction method, is predicted to be hydroxylated if the protein to which it belongs is expressed and secreted in a plant cell. In the claims, if no particular method is specified, then any disclosed method, or art-recognized method, may be used. Each disclosed method herein corresponds to a separate series of preferred embodiments, but the most preferred embodiments are those in which the standard quantitative prediction method, with the new matrix, is used.

The term “actual Pro-hydroxylation site” refers to a proline residue which in fact is hydroxylated if the protein to which it belongs is expressed and secreted in a plant cell.

The term “predicted Hyp-glycosylation site” means a proline residue which, according to the specified prediction method, is predicted to be hydroxylated to form hydroxyproline, and which hydroxyproline is predicted to be glycosylated, at least in part. In the claims, if no particular method is specified, then any disclosed method, or art-recognized method may be used. Each disclosed method herein corresponds to series of preferred embodiments, but the more preferred embodiments are those in which the new standard prediction method is used.

The term “actual Hyp-glycosylation site” means a proline residue which, in a protein expressed and secreted in a plant cell, in fact acts as a target site of plant cell hydroxylation (forming a hydroxyproline) and subsequent glycosylation. Such glycosylation need not be complete; a Hyp is considered an actual target site for plant cell glycosylation if at least 25% of the protein molecules are glycosylated at that position in at least one species of plant cell.

Predicted hydroxyproline (i.e., Pro-hydroxylation) sites are deemed to be non-contiguous but clustered if they are part of a series (i.e., two or more) of non-contiguous sites, wherein any site is separated from the nearest site, on either side, by one and only amino acid, and that separating amino acid is not a proline or hydroxyproline. Thus, the smallest possible cluster, other than at the N- or C-terminal, is of the form -X-O-X-O-X-, since the two O are non-contiguous, and separated by each other by one separating amino acid.

It follows that, in O-O-X-O-X-O-X-O-X-X-O-X-X (SEQ ID NO: 50), the third, fourth and fifth hydroxyprolines, which are boldfaced, are part of a single cluster of non-contiguous hydroxyprolines, while the first and second hydroxyprolines are a contiguous dipeptide block, and the final hydroxyproline is isolated (a hydroxyproline which is not part of a contiguous series, and not part of a cluster, is considered isolated).

On the other hand, O-O-X-O-X-O-O (SEQ ID NO: 51) does not feature a cluster, but rather two dipeptidyl Hyp with a lone unclustered Hyp in-between.

Clustered actual hydroxyproline sites are analogously defined.

Predicted Pro-hydroxylation or Hyp-glycosylation sites are deemed to be proximate to each other if there are no intervening prolines (or hydroxyprolines) and if they are separated by not more than four intervening amino acids which are not prolines or hydroxyprolines (e.g., O-X-X-X-X-O). Proximate actual Pro-hydroxylation or Hyp-glycosylation sites are analogously defined.

Sites of a particular kind (e.g., predicted Hyp) are said to be grouped if they are a series (i.e., two or more) of non-contiguous sites, each site is proximate to the next site in the series, and the sites don't satisfy the definition of clustered sites. Isolated sites may be grouped or not. If not grouped, they may be termed “highly isolated.”

As used herein, the term “predicted Hyp-glycomodule” is meant to refer to an amino acid sequence consisting of (1) an uninterrupted series of proximate predicted Hyp-glycosylation sites, (2) the amino acids, if any, between any two such Hyp-glycosylation sites of that series which are not themselves such Hyp-glycosylation sites, (3) the two amino acids, if any, before the first Hyp-glycosylation site of such series, and (4) the two amino acids, if any, after the last Hyp-glycosylation site of such series. For this purpose, predicted Hyp-glycosylation sites are said to be in series if the first site is proximate to the second, the second to third (if any), the third to the fourth (if any), and so on without any gap of more than four intervening amino acids which are not prolines or hydroxyprolines. Thus, a Hyp-glycomodule could be, e.g., X-X-O-O-X-O-X-X-O-X-X-X-O-X-X-X-X-O-X-X (SEQ ID NO: 52), assuming that all of the hydroxyprolines (O) are in fact Hyp-glycosylation sites, as the sequence then includes a series of six sites, each proximate to the next one. The term “actual Hyp-glycomodule” is analogously defined.

The term “Hyp-glycomodule” may be used not only to refer to the final processed form of the moiety, including one or more glycosylated hydroxyprolines, but also, more loosely, to refer to the amino acid sequence of the Hyp-glycomodule before it undergoes any post-translational modification, or to the sequence which is hydroxylated (and thus includes one or more hydroxyprolines), but those hydroxyprolines are unglycosylated or incompletely glycosylated. If it is necessary to distinguish these concepts, then the equilibrium glycosylated form may be referred to as the mature or final Hyp-glycomodule, the immediately expressed form, prior to hydroxylation or glycosylation, may be referred to as the nascent Hyp-glycomodule, and any intermediate form may be referred as an intermediate Hyp-glycomodule. The amino acid sequence of the nascent Hyp-glycomodule may be referred to as the initial core sequence thereof and the amino acid sequence of the final Hyp-glycomodule, with hydroxyprolines identified (but ignoring glycosylation), may be referred to as the modified core sequence thereof.

### Hyp-Glycosylation Types

Hyp-Glycosylation types include, but are not limited to, arabinosylation and arabinogalactan-polysaccharide addition. Arabinosylation generally involves the addition of short (e.g., generally about 1-5) arabinooligosaccharide (generally L-arabinofuranosyl residues) chains. Arabinogalactan-polysaccharides, on the other hand, are larger and generally are formed from a core β-1,3-D-galactan backbone periodically decorated with 1,6-additions of small side chains of D-galactose and L-arabinose and occasionally with other sugars such as L-rhamnose and sugar acids such as D-glucuronic acid and its 4-o-methyl derivative. Arabinogalactan-polysaccharides can also take the form of a core β-1,6-D-galactan backbone periodically decorated with 1,6-additions of small side chains of arabinofuranosyl. Note that these adducts are added by a plant's natural enzymatic systems to proteins/peptides/polypeptides that include the target sites for glycosylation, i.e., the glycosylation sites. There may be variation in the actual molecular structure of the glycosylation that occurs. The oligosaccharide chains may include any sugar which can be provided by the host cell, including, without limitation, Gal, GalNAc, Glc, GlcNAc, and Fuc.

### Prediction of Pro-Hydroxylation and Hyp-Glycosylation Sites

In general, methods of predicting Pro-hydroxylation and Hyp-glycosylation sites will strike a balance between the competing goals of simplicity and accuracy. Prediction rules which attempt to explain the patterns of hydroxylation and glycosylation for all known proteins, without exception, are likely to be too complex.

Moreover, a rule created to explain a single site in a single protein may invoke a feature which is actually irrelevant or only marginally relevant to the susceptibility of that site to hydroxylation and glycosylation, and hence lead, when applied to new proteins, to erroneous predictions. (This is sometimes referred to as “over-training” a rule to match a data set.)

Hence, any reasonable prediction rule will result in both false positives (saying it is hydroxylated or glycosylated, when in fact it isn't) and false negatives (saying it isn't, when in fact it is). For this reason, we have been careful to define both predicted and actual Hyp-glycosylation sites. Nonetheless, we believe that the current prediction methods are sufficiently accurate to be useful in designing systems for secreting biologically active proteins (or proteins cleavable to release biologically active proteins) in plant cells.

All predicted/actual Hyp-glycosylation sites are also, necessarily, predicted/actual Pro-hydroxylation sites, but not vice versa.

The present disclosure sets forth three methods for the prediction of proline hydroxylation. In one series of embodiments, the qualitative standard method is used. In a second and most preferred series of embodiments, the quantitative standard method, which generates a Hyp-score, is used. (This preferably uses the new standard matrix, but may alternatively use the old one.) In a third series of embodiments, the qualitative alternative method is used. These three series of embodiments overlap a great deal, but are not identical. The quantitative standard method may further be classified into subseries of embodiments depending on the choice of the three parameters of the method.

The present disclosure sets forth three methods for the prediction of hydroxyproline glycosylation: 1) the old standard method, 2) the old alternative method, and 3) the new standard method. In one series of embodiments, the new standard method is used. In a second, overlapping series of embodiments, the old standard method is used. There is further a subset in which the “extension” (dealing with isolated Hyp residues) is used, and a subset in which it isn't. In a third overlapping, series of embodiments, the alternative method is used.

While these methods attempt to predict the type of glycosylation which occurs at a particular residue, this is not as important as knowing whether glycosylation occurs at all.

The present program implementation of the methods for predicting hydroxylation and glycosylation doesn't include any subroutines for the prediction of signal peptidase cleavage sites. Consequently, if the sequence of the protein, as input into the program, includes the signal sequence, the program may predict Pro-hydroxylation sites and Hyp-glycosylation sites within the signal peptide. Moreover, residues in the signal sequence may be close enough to a Pro outside the signal sequence to influence the predictions made concerning that proline.

If Proline hydroxylation is co-translational, and thus begins before the signal peptide is cleaved, then signal peptide residues could conceivably affect the hydroxylation of nearby non-signal prolines (but not the glycosylation of nearby Hyp). However, we have noticed that the first Pro at the amino-terminal of our secreted synthetic test proteins (e.g., those with numerous SP repeats) is often not hydroxylated.

It is optional, but within the contemplation of the present invention, to add such subroutines, and to limit the input to the predictive method to the putative mature sequence. Alternatively, the full sequence can be input, and the location of the signal sequence may be taken into account when reviewing the predictions made.

Likewise, the programs don't include any subroutines for the prediction of GPI addition signals. Consequently, there could be prediction of Pro-hydroxylation or Hyp-glycosylation within or near the GPI addition signal, which might not be predicted if that signal were not within the inputted sequence. It is believed that GPI addition is post-translational, which implies that the GPI addition sequence (cleaved off, and the GPI anchor added, in the endoplasmic reticulum) can influence hydroxylation of nearby Pro, but not glycosylation of nearby Hyp.

If the protein under consideration is a naturally occurring protein which, in nature, is not secreted, then it shouldn't have GPI addition signals. Likewise, if it is a modified protein, if the parental protein, in nature, is not secreted, then it shouldn't have GPI addition signals (unless those are deliberately or fortuitously created by the modifications). Thus, GPI addition signals are primarily a concern in the case of naturally secreted proteins and modifications thereof.

It is optional, but within the contemplation of the invention, to include, at some stage, means for identifying GPI addition signals and, if desired, ignoring the part of the sequence which would be replaced by the GPI anchor.

### Prediction of Pro-Hydroxylation

### Qualitative Prediction of Proline Hydroxylation (Standard Method)

We have the following standard qualitative rules for predicting whether a proline is hydroxylated:

1. A proline immediately preceded by Lys, Ile, Gln, Arg, Leu, Phe, Tyr, Asp, Asn, Cys, Trp or Met is not hydroxylated.

2. A proline immediately preceded by Ala, Ser, Val, Thr or Pro is likely to be hydroxylated. This is even more likely to occur if the proline is both immediately preceded and immediately followed by one of those five amino acids, e.g., SPS, APS, TPA, APT, APA, APV, SPV, etc.

3. A proline immediately preceded by Glu, Gly or H is can be hydroxylated, but this is more sensitive to the nature of other amino acids in the vicinity of that proline.

A quantitative prediction method is set forth in the next section.

### Quantitative Prediction of Proline Hydroxylation (Hydroxyproline Formation), Standard Method

The standard quantitative prediction method draws upon, but goes beyond, the teachings of the qualitative method set forth in the last section. In particular, it considers the effects of residues which are not adjacent to the target proline.

For each proline in the protein, one may calculate a hydroxyproline (Hyp) score:

HypScore=(LCF/LCFB)*(MV),

where LCF is the Local Composition Factor Score, LCFB is the Local Composition Factor Baseline, and MV is the Matrix Value, all as defined below.

In preferred embodiments of the quantitative standard method, the proline is predicted to be hydroxylated if the HypScore is greater than the Score Threshold. The preferred (default) value of the Score Threshold is 0.5. A proline for which the Hyp Score thus calculated is greater than the Score Threshold is considered to be a predicted Pro-Hydroxylation Site for that Score Threshold. Such a site is a candidate for evaluation for hydroxyproline glycosylation, as described in a later section. For the purpose of the claims, if no LCFB or Score Threshold is specified in the claims, the preferred (default) values are assumed.

### Matrix Value

The Matrix value is the sum of the matrix scores, from the table below, for the amino acids in positions n−2, n−1, n+1 and n+2, where the target proline is at position n. If position n is so close to the amino or carboxy terminal that one or more of these positions is null, then the null position(s) can be given a matrix score of zero. However, we would recommend that the proteins of choice be ones for which at least one proline predicted to be hydroxylated and glycosylated is not within three amino acids of the amino or carboxy terminal, as the applicability of our algorithm to these extreme cases is less certain.

### Proline Hydroxylation Score Matrix:

| | Position Relative to Target Proline (−2, −1, 1, 2) | | | |
|---|---|---|---|---|
| | and Corresponding Position Values Used to Determine | | | |
| | Likelihood of Hydroxylation* | | | |
| Amino Acid | −2 | −1 | +1 | +2 |
| A | 1 | 3 | 3 | 0.5 |
| C | −8 | −8 | −5 | −8 |
| D | −1 | −8 | 0 | −2 |
| E | −1 | −0.5 | −0.1 | −0.5 |
| F | −2 | −8 | 0.1 | −1 |
| G | 1 | 0 | 1 | −0.6 |
| H | 1 | −5 | −0.3 | 1 |
| I | −0.5 | −8 | −0.5 | −0.5 |
| K | 0.5 | −8 | 1 | 1 |
| L | −0.5 | −8 | −0.5 | −0.5 |
| M | −0.5 | −8 | −0.5 | −0.5 |
| N | −0.5 | −8 | 0.5 | −2.5 |
| O | 2 | 3 | 2 | 1 |
| P | 2 | 3 | 3 | 3 |
| Q | −2 | −8 | −1 | −0.5 |
| R | −0.5 | −8 | 1 | −3 |
| S | 1 | 4 | 2 | 0.5 |
| T | 1.5 | 2 | 1 | 0.5 |
| V | 1 | 1 | 1 | 1 |
| W | −5 | −8 | −2.5 | −1 |
| Y | 1 | −8 | 0.5 | 0.5 |

The “new standard” matrix shown above differs slightly from the “old standard” one set forth in 60/697,337. Specifically, D (Asp) in position +1 was previously scored as −1 (now 0), and G (Gly) in position −1 was formerly scored as −0.75 (now 0). These changes make the scoring system more permissive, which should increase the number of both hits (correct prediction of hydroxylated prolines) and false positives (prolines predicted to be hydroxylated which aren't). In general, false positives are preferred to false negatives.

Preferably, the new standard matrix is used, and references to the matrix, without qualification, assume its use. However, in an alternative embodiment, the old standard matrix is used.

Please also consider the row beginning 0 (Hyp). This row is not part of the old or new standard matrix; its use is optional. In normal usage, the protein sequence is scanned only once, and hydroxylation is “applied” only after the scan is complete. Consequently, the flanking amino acids −2, −1, +1 and +2 can be Pro, but not Hyp. However, one can optionally conduct multiple scans, in which case those positions could be Hyp as a result of a previous iteration. Since the scores for Hyp at +1 and +2 are lower than those for Pro, this could lead to a reduction of the Hyp Score for some positions.

Comparing the matrix with the qualitative rules, we can see that the residues which are expected by rule 1 to block hydroxylation if they occur at position −1 are given matrix values of −8, and that the highest possible matrix score is then zero (sum of +2 −8 +3 +3).

The residues favored by rule 2 are assigned matrix values ranging from +1 to +4. Thus, depending on the nature of the residues at positions −2, +1 and +2, the matrix score can be negative or positive.

The matrix reveals that the nearby residues most likely to hinder hydroxylation, are, at the −2 position, Cys, Trp and Gln; at the +1 position, Cys and Trp; and at the +2 position, Cys, Asp, Asn and Arg.

The residues referred to by rule 3 are given, when they appear at the −1 position, matrix values of −0.5 (Glu), −0.75 (Gly), or −5 (His); i.e., they are considered unfavorable, but not as much as are the rule 1 residues. Note that Gly is favorable in the +1 position, so a GPG has a net, slightly favorable, partial matrix score.

Rule 4 is not considered directly in the present version of the quantitative method, except to the extent that if the Cys in question is within two amino acids of the proline, it has a strongly unfavorable effect on the matrix score.

### Local Composition Factor: Entropy and Order

Pro hydroxylation is common in proteins and regions of proteins that are highly repetitive and rich in Pro/Hyp (therefore less random); Pro hydroxylation is less likely in those that are not repetitive.

In signal theory, Shannon entropy is defined as the sum of the −(pᵢ log₂ (pᵢ)) for all signals i for which pᵢ>0, where pᵢ is the probability of occurrence of signal i, where the signal i is either yes or no (i.e., a binary channel). In applying this entropy measure to sequence analysis, the pᵢ are the proportions of amino acids in a sequence which are a particular type i of amino acid (e.g., proline, or leucine, or glycine). Thus, in a normal protein, up to twenty types may be represented. Thus, we define the absolute entropy score for an amino acid sequence as being the Shannon entropy, with the pᵢ calculated as explained above. In calculating the absolute entropy score for a protein sequence, we ignore post-translational modifications, such as Pro to Hyp, or glycosylation.

Repetitiveness is a form of order, and the entropy score is a formal mathematical measure of disorder. The repetitiveness of the protein sequence is evaluated in a window around the target proline, so the entropy is a measure of the repetitiveness of the protein in a region localized around the target proline, rather than that of the protein as a whole (unless the window is large enough to include the entire protein).

It should be noted that the entropy calculated in this manner is an incomplete measure of repetitiveness in the sense that it only considers the amino acid composition of the sequence, and not the ordering of the amino acids within it, so a sequence in which two amino acids alternate would have the same Shannon entropy as a random sequence which is 50% one and 50% the other.

If a protein sequence was a homopolymer, i.e., all the same amino acid, then the absolute entropy score would be zero. That is the smallest possible value. If a protein sequence had an equal number of each of the twenty possible amino acids (we will call this an equipolymer), the absolute entropy score would be −log₂ ( 1/20), or 4.32198, which is the maximum entropy for an amino acid sequence.

We can then define the following:

absolute order=maximum entropy−absolute entropy score

relative entropy=absolute entropy score/maximum entropy

relative order=absolute order/maximum order
(maximum order equals the maximum entropy, since the minimum absolute entropy score is zero)

The Local Composition Factor is the relative order as defined above, and it is normally evaluated over a window centered on and including the target Proline. The window may be an odd or an even number of amino acids. If it is an odd number, and the position of the target proline is denoted n, then the normal window is from position n−a to position n+a, where a is the (width−1)/2, and the width is 2a+1. If the window is even in size, then the window can be defined in two ways, either from position n−a to position n+a−1, or from position n−a+1 to position n+a, where a is the half-width, so the width is 2a. The preferred standard window size is 21 amino acids, so the preferred standard window is from n−10 to n+10.

When the target proline is close to the amino acid or carboxy terminal of the protein of interest, the window will be truncated on that side of the proline, reducing the effective window size. For example, if we were using a standard window size of 21 amino acids, but the target proline were at the amino terminal, then the “left half” of the window would be truncated, reducing the effective window size to 11, and the Local Composition Factor would be calculated over positions 1-11 of the protein.

Note that when the effective window size is less than 20, it is impossible to achieve the maximum entropy since it is impossible for all twenty amino acids to be present in the effective window.

The Local Composition Factor Baseline (LCFB) is the value of the Local Composition Factor (LCF) for which the effect of the local composition on hydroxylation of prolines, measured as described above, is considered to be neutral. The preferred (default) value is 0.4.

Comparison with Shimizu

It is interesting to compare the standard method quantitative scoring algorithm to the consensus sequence of Shimizu. Shimizu says that hydroxylation of proline requires the five amino acid sequence
Xaa1-Pro-Xaa3-Xaa4-Xaa5 where
   where Xaa1 is Ala, Val, Ser, Thr or Gly,
   Xaa3 is Ala, Val, Ser, Thr, Gly or Ala [sic],

### Xaa4 is Gly, Ala, Val, Pro, Ser, Thr or Cys, and

Xaa5 is Ala, Pro, Ser or acidic (Asp or Glu)

Our matrix score ignores Shimizu's Xaa5 position, and Shimizu ignores the residue at the n−2 position relative to the proline at n. Someone following Shimizu's teaching could have an n−2 residue with a matrix value anywhere from −8 (Cys) to +2 (Hyp, Pro). H is n−1 residues (Xaa1) have matrix values ranging from −0.75 (Gly) to 1.5. H is n+1 residues range from 1 to 3. H is N+2 residues range from −0.6 (Gly) to 3 (Pro). Hence, the Prolines predicted by Shimizu to be hydroxylated could have matrix scores, according to our algorithm, ranging from −6.6 to +9.5. Shimizu does not consider the entropy of the larger sequence environment, which further increases the variability in our scoring of proline-containing sequences which Shimizu would predict to be modified.

It is also interesting to inquire into the highest matrix score possible for a sequence which does not satisfy Shimizu's consensus sequence. These sequences fall into two categories.

First, there are those for which Shimizu's Xaa5 criterion is not satisfied. Our matrix score does not consider Shimizu's Xaa5 position at all.

Secondly, there are those for which Shimizu's Xaa1, Xaa3 and/or Xaa4 criteria are violated. Shimizu does not consider the n−2 position, at which the matrix score could be as high as 2. At Xaa1 (our n−1), Shimizu ignores the possibility of Pro, which we would score as +3. At Xaa3 (our n+1), Shimizu ignores the positive scoring Phe (+0.1), Lys (+1), Hyp (+2), Pro (+3), Arg (+1), and Tyr (+0.5). At Xaa4 (our n+2), Shimizu ignores the positive scoring H is (+1), Lys (+1), and Tyr (+0.5).

Note also that we could tolerate a negative scoring AA at Xaa1, Xaa3 or Xaa4 if the other positions compensated. If the LCF equals the LCFB, then we would predict a target proline to be hydroxylated if its matrix value (the sum of the four matrix scores) exceeded 0.5. For example, if the target proline were preceded by SE and followed by SV, the Matrix Value would be (+1)+(−0.5)+(+2)+(+1)=3.5, even though the residue at Xaa1 was the negative scoring Glu (E).

Hence, a class of embodiments of interest are those proteins in which at least one proline is predicted to be hydroxylated by our algorithm, even though that proline would not be predicted to be hydroxylated on the basis of Shimizu's consensus sequence. (We are presently uncertain whether Shimizu considers Asn and Gln to be acidic residues in reference to Xaa5 above. Hence, there are two contemplated subclasses, one in which we assume that they are allowed by Shimizu at Xaa5, and another in which we assume that they aren't.) Of particular interest are those proteins in which at least one proline is predicted to be hydroxylated by our algorithm, even though none of the prolines in that protein satisfy Shimizu's consensus sequence.

The present computer implementation of the quantitative method doesn't take the species of plant cell into account, i.e.,

GP is not hydroxylated in Acacia or tobacco, but is in Arabidopsis

HP is not hydroxylated in the solanaceae (e.g., tobacco, tomato, eggplant, nightshade, peppers) but is in maize and probably other graminaceous monocots

EP is partially hydroxylated in potato.

Instead, in the −1 position, G has a matrix weight of 0 (neutral), H of −5 (strongly unfavorable), and E of −0.5 (slightly unfavorable). That means that the computer program will tend to overlook, e.g., HP which would be hydroxylated in a suitable plant cell.

### Prediction of Pro-Hydroxylation, Alternative Method

We have the following alternative qualitative rules for predicting whether a proline is hydroxylated:

1. A proline immediately preceded by Lys, Ile, Gln, Arg, Leu, Phe, Tyr, Asp, Asn, Cys, Trp, Met, or Glu (i.e., they are in the −1 position) is not hydroxylated. A proline immediately preceded by Gly is hydroxylated in Arabidopsis, but not in Solanaceae or Leguminaceae. A proline immediately preceded by His is usually not hydroxylated, but there is at least one exception (in maize).

2. A proline immediately preceded by Ala, Ser, Thr or Pro is likely to be hydroxylated. However, the sequence PPP (as in SPPP) is incompletely hydroxylated in tobacco, presumably because it is very rare in tobacco HRGPs and not a favored substrate for prolyl hydroxylase.

3. Pro in the sequence Pro-Val is always hydroxylated unless hydroxylation is forbidden by rule 1.

Note that these alternative rules do not make any predictions as to the effect of the amino acids Val and Gly in the −1 position. If the alternative rules are used, then Val and Gly would be considered superior to the alternative rule 1 amino acids (which are clearly unfavorable) but inferior to the alternative rule 2 amino acids (which are clearly favorable).

### Comments

The folding of a protein may be such as to occlude potential Pro-hydroxylation sites. This is most likely to be a problem with proteins which have significant tertiary or supersecondary structure. Indicators of potential problem proteins are the presence of disulfide bonds (which may be inferred from the presence of paired cysteines) and low proline (proline tends to interfere with the formation of secondary structures such as alpha helices and beta strands, and hence with formation of higher structures).

While there are tools for predicting secondary, supersecondary and tertiary structure, the worker in the art may prefer to simply express the protein of interest in plants to determine whether the predicted Pro-hydroxylation sites are in fact hydroxylated.

### Significance of Predicted Pro-Hydroxylation Sites

Pro-hydroxylation sites are preferably predicted, as described above, on the basis of the Hyp-score. The number of predicted Pro-hydroxylation sites is then dependent on the choice of values in the Hyp-Score calculation for the LCFB, taken together with the Score Threshold, which determines whether the target proline is classified as a predicted Pro-hydroxylation site. Only predicted Pro-hydroxylation sites can be predicted Hyp-glycosylation sites. If the LCFB is given its preferred value as set forth above, then the number of predicted Pro-hydroxylation sites will be inversely (but not necessarily linearly) dependent on the Score Threshold.

Preferably, the prediction of Pro-hydroxylation sites (and thus, of candidate Hyp-glycosylation sites) is based on the preferred Score Threshold of 0.5. This value was found to yield acceptable results in predicting the hydroxylation of a “problem set” of weakly hydroxylated proteins. However, it is within the contemplation of the invention to predict Pro-hydroxylation and Hyp-glycosylation sites, and consequently to identify Hyp-glycosylation-predisposed and Hyp-glycosylation proteins, and to design Hyp-glycosylation-supplemented mutant proteins, on the basis of a different Score Threshold, such as 0.4, 0.45, 0.55 or 0.6.

It is within the contemplation of the invention to mutate a protein so as to improve the Hyp-score of one or more of the predicted Hyp-Glycosylation sites, rather than to create a new Hyp-Glycosylation site. Whether a mutation merely improves the Hyp-Score of a predicted site, or creates a new site, is dependent on the Score Threshold. For example, if a parental protein has four prolines, with Hyp scores of 0.6, 0.71, 0.83, and 1.2, and mutation increases the lowest score from 0.6 to 0.7, then there is an increase in the number of Pro-hydroxylation sites if the Score Threshold is 0.7, but not if the Score Threshold is 0.5. Thus, the improvement of the Hyp-Score of a Pro-hydroxylation site predicted with the default Score Threshold can be characterized as equivalent to the creation of a new predicted Pro-hydroxylation site if a more stringent Score Threshold is employed.

### Prediction of Hyp-Glycosylation

By designing and characterizing our own very simple HRGPs possessing repeats of only one putative Hyp-glycosylation glycomodule, we were able to determine that AOAOAOA (SEQ ID NO:53) and SOSOSOS (SEQ ID NO:54) repeats are exclusive sites of arabinogalactan addition to Hyp and that as soon as the Hyp became contiguous, as in SOOSOOSOO (SEQ ID NO:55), the Hyp glycosylation switched to arabinosylation only.

We found that the peptide structural isomers, Lys-Pro-Hyp-Val-Hyp (SEQ ID NO:56) and Lys-Pro-Hyp-Hyp-Val (SEQ ID NO:57), which differ only in Hyp contiguity, had marked differences in Hyp arabinosylation. Lys-Pro-Hyp-Val-Hyp is arabinosylated 20% of the time on the second Hyp residues. Lys-Pro-Hyp-Hyp-Val is always arabinosylated at Hyp residue 1. We also found that the peptide Ile-Pro-Pro-Hyp (SEQ ID NO:58) was not glycosylated. We found no arabinogalactosylation of any Hyp residues in this protein despite it having instances of clustered non-contiguous Hyp in the major repeat motif:

### Lys-Pro-Hyp-Val-Hyp-Val-Ile-Pro-Pro-Hyp-Val-Val-Lys-Pro-Hyp-Hyp-Val-Tyr-Lys-Pro-Hyp-Val-Hyp-Val-Ile-Pro-Pro-Hyp-Val-Val-Lys-Pro-Hyp-Hyp-Val-Tyr- . . . (SEQ ID NO:59)

(see Kieliszewski, M. J., de Zacks, R., Leykam, J. F., and Lamport, D. T. A. (1992) A repetitive proline-rich protein from the gymnosperm Douglas Fir is a hydroxyproline-rich glycoprotein. Plant Physiology, 98: 919-926.)

One wonders why PRPs, like the one above, are at best lightly arabinosylated but not arabinogalactosylated despite having some clustered non-contiguous Hyp. An examination of protein sequence and composition provides clues. Both PRPs and AGPs are Hyp-rich. However AGPs are also rich in Ala, Ser, Thr, and sometimes Gly, but notably in Tyr and Lys, at least in the Hyp-rich domains . . . and AGPs are not highly repetitive. PRPs are the most repetitive of the HRGPs and rich in Hyp, Val, Tyr, and Lys and seldom contain Ala or Gly. The most common repeat motifs of PRPs are variations of the pentapeptide/hexapeptide: Lys-Pro-Hyp-Val-Tyr/Lys-Pro-Hyp-Hyp-Val-Tyr (SEQ ID NO:60).

These general principles hold for extensins, too, which are highly arabinosylated HRGPs that contain some lone Hyp residues, as in the common sequence: Ser-Hyp-Hyp-Hyp-Hyp-Thr-Hyp-Val-Tyr-Lys (SEQ ID NO:61).

Like the PRPs, Extensins are highly repetitive (Ser-Hyp-Hyp-Hyp-Hyp, SEQ ID NO:62, is the extensin identifying sequence), Lys, Tyr, Val-rich, generally Ala and Gly-poor. Extensins are not arabinogalactosylated.

### Prediction of Hyp-Glycosylation, Old Standard Method

1. Hyp in blocks of three or more contiguous Hyp (“large block Hyp”) are about 100% arabinosylated.

2. Hyp in blocks of only two contiguous Hyp (“dipeptidyl Hyp”) are about 50-65% arabinosylated.

3. Non-contiguous Hyp residues can be arabinosylated, arabinogalactosylated, or non-glycosylated, as predicted by the rules below.
3.1. If the Hyp residues are Clustered Hyp residues (e.g., (X-Hyp)n, where X=Ser, Ala, Thr, Val or Gly and n>1), then
   3.1.1. they are arabinogalactosylated if the sum of Tyr, Lys and H is residues within the 11 amino acid window running from position −5 to position +5 (the target hydroxyproline being position 0) is zero or one.
   3.1.2. If condition 3.1.1 is not met, they are arabinosylated or non-glycosylated, and it is prudent to assume that they are non-glycosylated
3.2 If the Hyp residues are isolated Hyp residues then
   3.2.1. they are arabinogalactosylated if, within the aforementioned 11 amino acid window, all of the following conditions are met:
   (a) Hyp+Pro residues is less than 4;
   (b) Ser+Thr+Ala residues is greater than 3;
   (c) the number of different types of amino acids is greater than three OR Ser+Thr+Ala is greater than 4, e.g., SOOAAOAAAOS (SEQ ID NO: 63), in which the target hydroxyproline is boldfaced, there are only three types of amino acids in the window, but S+T+A=7, so (c) is met); and
   (d) the Hyp residue is not immediately followed by Lys, Arg, His, Phe, Tyr, Trp, Leu or Ile.
   3.2.2 otherwise, they are either arabinosylated or non-glycosylated.

If condition 3.2.2 applies, then the following method may be used to predict whether the Hyp is arabinosylated or not, but it should be noted that this extension is considered less accurate than the method as described up to this point. In essence, if condition 3.2.2 applies, the Hyp are non-glycosylated if at least two of the four conditions below are met for the aforementioned 11 amino acid window:

i) Hyp+Pro greater than 5;

ii) Ser+Thr+Ala less than 5;

iii) number of different types of amino acids less than 5; and

iv) Tyr+Lys greater than 1.

It will be appreciated that if the target proline is within five amino acids of the amino or carboxy terminal, the window will be truncated on the terminal side.

If the goal is to estimate the total number of glycosylated Hyp, rather than to identify which Hyp sites are glycosylated, then instead of applying this extension, 20% of the isolated Hyp may be assumed to be arabinosylated. See Kieliszewski et al., J. Biol. Chem., 270:2541-9 (1995).

### Comment:

Dipeptidyl Hyp: Our earlier work (Shpak et al 2001, J. Biol. Chem. 276, 11272-11278) with repetitive Ser-Hyp-Hyp motifs, which necessarily include dipeptidyl Hyp, indicated the first Hyp in the dipeptide block is always arabinosylated and the second one is incompletely arabinosylated.
The old standard method classifies all Hyp residues as large block Hyp, dipeptidyl Hyp, clustered Hyp or isolated Hyp. It may be advantageous to recognize a spectrum of isolation, e.g.,

### XXOXX*XXOXX

### XXXOXXX*XXXOXXX

### XXXXOXXXX*XXXXOXXXX

### XXXXXOXXXXX*XXXXXOXXXXX

Note that in the first three lines, the hydroxyprolines form a series of three (including the target Hyp) proximate Hyp, and are therefore considered “grouped”, while in the fourth line, the three hydroxyprolines are not proximate to each other and therefore are considered highly isolated.
We would expect grouped Hyp to be more likely to be glycosylated than would be highly isolated Hyp.
It is straightforward to synthesize simple diheteropolymeric polypeptides consisting essentially of repetitions of such sequences, e.g., repetitions of OXX, OXXX, OXXXX or OXXXXX with X being the same throughout the peptide (e.g., X=Ser, or X=Thr, etc.), in order to determine the effect of spacing of isolated Hyp residues on their glycosylation propensities.

### Prediction of Hyp-Glycosylation, Old Alternative Method

This old alternative method is much simpler than the old standard method.

1. Hyp in blocks of three or more contiguous Hyp are about 100% arabinosylated.

2. Hyp in blocks of only two contiguous Hyp (“dipeptidyl Hyp) are about 50-65% arabinosylated.

3. Hyp which are not contiguous with other Hyp are arabinogalactosylated.

### Prediction of Hyp-Glycosylation, New Standard Method

After predicting which prolines are hydroxylated to form hydroxyproline, we predict which hydroxyprolines are arabinosylated, galactoarabinosylated, or left “unaltered” (unglycosylated). We predict whether a particular Hyp will be glycosylated by considering a window of 11 consecutive residues centered on that Hyp. For the purposes of the algorithm described below, consider the residues of the window to be numbered 0-10, i.e., number 5 is the center. Also, note that whenever a summation is required, the “target Hyp” at position 5 of the window is ignored; i.e., the summation is over residues 0-4 and 6-10 of the window.

Test A: If residue 4 is Hyp then do test B, otherwise do Test C.

Test B: If residue 6 is Hyp OR residue 3 is Hyp then return an answer of Arabinosylated for residue 5. Otherwise return an answer of unaltered Hydroxyproline for residue 5. End all tests for this window.

Test C: If residue 6 is Hyp return an answer of Arabinosylated for residue 5 and end all tests for this window, otherwise do Test D.

Test D: If residue 3 is Hyp or Pro AND residue 2 is not Hyp then do test E, otherwise do test G.

Test E: If residue 4 is one of (Ser, Ala, Val or Gly) AND the total number of (Lys, Tyr, His) is fewer than two then return an answer of Arabinogalactosylated for residue 5, otherwise do test F.

Test F: If residue 4 is Thr then return an answer of Arabinosylated for residue 5, otherwise return an answer of unaltered Hydroxyproline for residue 5. End all tests for this window.

Test G: If residue 7 is Hyp or Pro AND residue 8 is not Hyp do test E, otherwise do test H.

Test H: If residues 4 to 6 inclusive have the one of the sequences (Thr-Hyp-Lys), (Thr-Hyp-His), (Gly-Hyp-Lys) or (Ser-Hyp-Lys) then return an answer of Arabinosylated for residue 5, otherwise do test I.

Test I: If residue 7 or residue 3 is Pro do test J, otherwise do test K.

Test J: If residue 4 is one of (Ser, Ala, Val or Gly) AND residue 6 is one of (Leu, Ile, Glu or Asp) then return an answer of Arabinogalactosylated for residue 5, otherwise do test K.

Test K: If residue 6 is one of (Lys, Arg, His, Phe, Tyr, Trp, Leu or Ile) then return an answer of unaltered Hydroxyproline for residue 5, otherwise do test L.

Test L: If the total number of (Hyp, Pro) is greater than three then return an answer of unaltered Hydroxyproline for residue 5, otherwise do test M.

Test M: If the total number of (Ser, Thr, Ala) is fewer than four then return an answer of unaltered Hydroxyproline, otherwise do test N.

Test N: If the total number of different residue types is greater than three then return an answer of Arabinogalactosylated for residue 5, otherwise do test O.

Test O: If the total number of (Ser, Thr, Ala) is greater than four then return an answer of Arabinogalactosylated for residue 5, otherwise return an answer of unaltered Hydroxyproline for residue 5. End all tests for this window.

### Discussion:

Tests A-C deal with contiguous Hyp. If the scan encounters O*O, OO*, or X*O (where * is the target Hyp, O is other Hyp, and X is another amino acid), these tests predict that * is arabindsylated. Note that X*O could mean either the beginning of 3+ block of Hyp, or the first Hyp of dipeptidyl Hyp. If it encounters XO*X it predicts that the * (the second Hyp of dipeptidyl Hyp) is left unglycosylated. Thus, the subtle difference between new standard tests A-C and rule 2 of the old standard method is that for dipeptidyl Hyp, the old method said that the dipeptide was about 50% arabinosylated, while the new method identifies the first Hyp as arabinosylated and the second as non-glycosylated.

The remaining tests of the new standard method relate to non-contiguous Hyp (X*X).

If test D is satisfied, we have a clustered non-contiguous Hyp/Pro sequences (specifically, X(O/P)X*X), and are directed to tests E and possibly also F. Arabinogalactans are associated with such sequences when they are Ala, Ser, Val, Gly rich and Lys, Tyr, His poor.

Test E looks to whether there is A/S/V/G preceding *, and whether the window in general is K/Y/H poor. If so, then the * (which is the second, or later, Hyp of a cluster) is predicted to be arabinogalactosylated.

While Thr can also promote arabinogalactan addition in this situation (as we have observed in tobacco cells expressing a repetitive TP synthetic sequence), and is common in AGPs, it was excluded from Test E because it doesn't appear to have the same effect in maize. The person skilled in the art may wish to modify the algorithm to account for differences between, e.g., dicots like tobacco, and graminaceous monocots like maize. That is part of the test in view of, e.g., the lack of arabinogalactosylation of * in certain X(O/P0T*X sequences in, maize THRGP (CAA45514) and maize-expressed human IgA1.

If test E is failed, the complementary test F predicts arabinosylation of * in X(O/P)T*X.

In combination, tests E and F predict arabinosylation, but not arabinogalactosylation, of certain T*X sequences, consistent with N. tabaccum extensin (JU0465), maize THRGP (CAA45514) and maize-expressed human IgA1.

(It might be profitable to instead specify that Hyp in T*X in maize and other Graminae can only be arabinosylated, while allowing arabinogalactan addition if the T*X is expressed in a non-graminaceous species.)

If test D is failed, we go to test G. If test G is satisfied, we reach test E by a new route. The prior failure of test D means that the * is the first Hyp of a cluster. Satisfaction of test E means that it is arabinogalactosylated. Test G was inspired by LeAGP-1 and the sequence HSOLPT (SEQ ID NO: 64) in Jay's gum, wherein the SOLP (Aas 1-4 thereof), while of the form XOXP, behaves much like XOXO.

Tests D-G of the new method deal, as did old rule 3.1, with clustered Hyp residues. However, unlike the old rule, they don't accept T*X. That is a problem with certain maize THRGP sequences, so test H, if satisfied, predicts arabinosylation of the * in the sequences T*K, T*H, G*K and S*K.

Tests I through K distinguish among AGP-like sequences having clustered Pro/Hyp, and PRP/extensin sequences having clustered Pro/Hyp.

Tests J and K deal with unique modules in ‘problem proteins’ like Jay's Gum and THRGP from Maize, which was a particular problem. Test J was designed for test case ‘Jay's Gum’ (AKA [Gum-I]n in the paper: M J Kieliszewski and J Xu, “Synthetic Genes for the Production of Novel Arabinogalactan-proteins and Plant Gums,” Foods and Food Ingredients Journal of Japan, 211 (1): 32-36. (2006). Ile, Glu and Asp were added, speculatively as amino acids following Pro that are likely to allow arabinogalactosylation. Test K surveys composition in similar sequences and determines that when the target Hyp is followed by bulky amino acids like Lys, H is, Tyr, I, F, L (at residue 6) the Hyp remains non-glycosylated. R, W were thrown in for cases that might arise although these amino acids are rare in HRGPs.Gum Arabic Glycoprotein is one example; it contains the sequence TOOTG*HSOSOA (SEQ ID NO:43), with target Hyp shown as *. The O in GOH is not arabinoglycosylated.

Test L-O deal with the situation of isolated Hyp residues, as did old 3.2. Tests L-M are defined so that if either are positive, the target Hyp is unaltered. On the other hand, tests N and O are defined so that if either is positive, the target Hyp is arabinogalactosylated.

The old standard says that if all of 3.3.1(a)-(d) are positive, then the target Hyp is arabinogalactosylated. Whereas if any are negative, then by 3.2.2 the target Hyp is unaltered. (Ignoring the extension to 3.2.2 which accounts for the possibility of arabinosylation).

If we reach test L, we know that old 3.3.1(d) is negative, because if old 3.3.1(d) were positive, then test K would have been positive and unaltered target Hyp predicted.

Tests L-O are related to old rule 3.2, as follows: if old 3.2.1(a) is negative, test L is positive; if old 3.2.1(b) is negative, test M is positive; and if old 3.2.1 (c) is positive, test N and/or test O are positive.

### Evaluation

In developing the preferred Pro-Hydroxylation and Hyp-glycosylation predictive methods, we considered amino acid sequences (see Reference List H below for citations) of characterized HRGPs, i.e. those where both the proline hydroxylation and Hyp glycosylation profiles had been experimentally determined. This included extensins from tomato, Asparagus, Douglas fir, sugar beet, tobacco, Gingko, Maize and melon; PRPs from Douglas fir and soybean, and AGPs from Acacia senegal and tobacco, and a tomato systemin. We then tested the accuracy of the Hyp Predictor by comparing its predictions with three recently characterized HRGPs [REF] from Arabidopsis, namely: At1g21310 (an extensin), At1g28290 (an AGP chimera), and At4g31840 (a small AGP similar to an early nodulin). These weren't part of the training set used to devise the methods. The table below shows its performance on those proteins, as well as on representative cases of the major classes of proteins with native Hyp-glycomodules.

TABLE
The Hyp content and Hyp glycosylation profiles of characterized HRGPs compared
with estimations made by the default method, implemented in a computer program.

| | Mol % | Mol % | % Hyp- | % Hyp- | % Hyp- | % Hyp- | % Hyp | % Hyp |
|---|---|---|---|---|---|---|---|---|
| | Hyp | Hyp | PS | PS | Ara | Ara | Gly | Gly |
| Sample | Pred | Meas | Pred | Meas | Pred | Meas | Pred | Meas |
| Arabidopsis | | | | | | | | |
| At1g21310 | 39 | 30 | 0 | 3 | 99 | 80 | 99 | 83 |
| At1g28290 | 16 | 16 | 2 | 43 | 9 | 52 | 11 | 95 |
| At4g31840 | 5 | 5 | 71 | 92 | 14 | 0 | 85 | 92 |
| Maize THRGP | 36 | 25 | 1 | 0 | 48 | 52 | 49 | 52 |
| CAA31854 | | | | | | | | |
| Tobacco P1 | 39 | 36 | 0 | 0 | 70 | 90 | 70 | 90 |
| S33158 | | | | | | | | |
| Tobacco (TP)₁₀₁ | 53 | 37 | 0 | ~60 | 100 | ~29 | 100 | ~89 |
| (SEQ ID NO: 70) | | | | | | | | |
| Synthetic gene | | | | | | | | |
| product | | | | | | | | |
| Tomato LeAGP-1 | 24 | 29 | 50 | 54 | 24 | 33 | 74 | 87 |
| CAA67585.1 | | | | | | | | |

PS = polysaccharide (i.e., arabinogalactosylation), Ara = arabinosylation, Gly = glycosylation (sum of PS and Ara).

It should be noted that for the purpose of the present invention, what is most important is that it correctly predicts that a protein will exhibit some degree of Hyp-glycosylation. It is less important that it predicts the exact number of actual Hyp-glycosylation site. If a protein is predicted to contain one or more Hyp-glycosylation sites, then one would generally want to try expressing and secreting it in plant cells before going to the trouble of mutating it to create additional Hyp-glycosylation sites (or improve the existing ones).

### Meaning of “Predicted”

The term “predicted”, as applied to a Pro-Hydroxylation or Hyp-Glycosylation site, is not intended to imply that the prediction must actually have been made prior to the expression and secretion of the protein in plant cells. Rather, it means that the site is predictable to be a such a site. The only exception would be in the context of a claim which explicitly recites a prediction step occurring before the expression step.

### Number of Predicted and Actual Hyp-Glycosylation Sites

While a protein with predicted Hyp-glycosylation sites, and no actual Hyp-glycosylation sites, may be biologically active, and hence useful, it is highly desirable that the proteins of the present invention have at least one actual Hyp-glycosylation site.

The number of actual Hyp-glycosylation sites should be sufficient to achieve the desired levels of secretion in plant cells. It does not appear that the level of secretion increases as a smooth function of the number of actual Hyp-glycosylation. The non-plant proteins with addition glycomodules featuring as few as two and as many as over one hundred Hyp-glycosylation sites have demonstrated increased secretion. It is believed that even a single site can provide at least an improved level of secretion.

Nonetheless, it is desirable to provide proteins with more than one actual Hyp-Glycosylation site, to provide greater assurance that the threshold required for increased or high level secretion is reached. Thus, the number of actual Hyp-glycosylation sites may be one, two, three, four, five, six, seven, eight, nine, ten or more, such as at least fifteen, at least twenty, etc.

The main limitation on the number of actual Hyp-glycosylation sites is that the level of Hyp-glycosylation not so great as to substantially interfere with expression, e.g., through excessive demand for sugar for incorporation into the glycoprotein. Preferably the number of actual Hyp-glycosylation sites is not more than 1000, more preferably not more than 500, still more preferably not more than 200, even more preferably not more than 150, and most preferably not more than 100. That said, proteins with addition Hyp-glycomodules featuring as many as 160 Hyp-glycosylation sites have been expressed and secreted in plants.

In some embodiments, all of the predicted Hyp-glycosylation sites are actual Hyp-glycosylation sites. In other embodiments, only some of them are actual Hyp-glycosylation sites, the others being false positives. Whether a predicted site is an actual site may in fact vary depending on the species of plant cell, as there are differences in hydroxylation and perhaps also glycosylation patterns, depending on the species. There may also be one or more false negatives (unpredicted actual Hyp-glycosylation sites).

In general, the goal is to achieve a particular number (or range of numbers) of actual Hyp-glycosylation sites. The desired number of predicted Hyp-glycosylation sites will then depend on the propensity of the Hyp-glycosylation prediction method toward false positives and negatives. For example, if you wanted to achieve at least two actual Hyp-glycosylation sites, and the prediction method was such that there was a 50% chance that the predicted Hyp-glycosylation site was a false positive (and there was a 0% chance of a false negative), then you would want at least four predicted Hyp-glycosylation sites.

Predicted Hyp-glycosylation site may vary in terms of the probability that they are actually glycosylated, and the prediction method may be devised so as to state such a probability for each site.

For a site to be an actual Hyp-glycosylation site, it must also be an actual Pro-Hydroxylation site. Hence, to achieve a particular number of actual Hyp-glycosylation sites, the protein must have at least that number of actual Pro-Hydroxylation sites.

In like manner, for a site to be a predicted Hyp-glycosylation site, it must also be a predicted Pro-hydroxylation site. However, bear in mind that predicted Pro-hydroxylation sites may vary in terms of the probability that the prolines in question are in fact hydroxylated, and the prediction method may be devised so as to state a probability for each site. The Hyp-Score referred to above is believed to be related to that probability, with a high score indicating a high probability of hydroxylation.

To achieve a particular number of predicted Hyp-glycosylation sites, you will generally need an equal or greater number of predicted Pro-hydroxylation sites.

### Experimental Determination of the Existence, or the Total Number, of Actual Pro-Hydroxylation and Hyp-Glycosylation Sites.

The existence, or the total number, of the actual Pro-Hydroxylation sites and of the actual Hyp-glycosylation sites may be determined by any suitable method.

We determine the Hyp-O-glycosylation profiles of hydroxyproline-rich glycoproteins (HRGPs); whether naturally occurring or products of synthetic gene expression, as previously described. Lamport, D. T. A. and D. H. Miller. “Hydroxyproline arabinosides in the plant kingdom.” Plant Physiol. 48: 454-56 (1971).

Unlike the serine and threonine O-glycosylation which are base-labile linkages (the glycans are attached to a β-carbon and β-eliminate in base), the glycosyl-Hyp linkage is base-stable. Thus base hydrolysis of a protein O-glycosylated through Hyp residues gives rise to a mixture of amino acids and Hyp-glycosides (the peptide bonds, but not the Hyp-glycosyl linkages, are broken).

The free amino acid Hyp and the Hyp occurring in Hyp-glycosides can be calorimetrically assayed and the amount of Hyp in a protein thereby quantified after base or acid hydrolysis of that protein (Hyp assays), see Kivirikko, K. I. and Liesmaa, M., “A colorimetric method for determination of hydroxyproline in tissue hydrolysates,” Scand. J. Clin. Lab. Invest. 11:128-131 (1959). The assay involves opening of the Hyp ring by oxidation with alkaline hypobromite, subsequent coupling with acidic Ehrlich's reagent and monitoring absorbance at 560 nm.

We quantify the relative abundance of each Hyp-glycoside and non-glycosylated Hyp in a protein by base hydrolysis of the protein, fractionation of the hydrolysate on a C2-Chromobeads strong cation exchange resin equilibrated in water and eluted with an acid gradient. The cation exchange column separates the amino acids including the Hyp-glycosides, which elute from the column in order, the largest first and non-glycosylated Hyp last. Individual fractions can be collected and assayed manually for Hyp using the colorimetric assay. Alternatively, we have automated the process which allows constant colorimetric monitoring of the post-column eluate by combining the eluate with the alkaline hypobromite and Ehrlich's reagent automatically. A flow-through spectrophotometer attached to a chart recorder records the flow at 560 nm. The peak response at 560 nm is directly related to the amount of Hyp in that peak. Integration of the area of the 560 nm-absorbing peaks (only Ehrlich's-coupled Hyp absorbs at 560 nm) allows us to determine the relative abundance of the Hyp-glycosides: Hyp-arabinogalactan polysaccharide, Hyp-Ara₄, Hyp-Ara₃, Hyp-Ara₂, Hyp-Ara, and non-glycosylated Hyp.

The number of Hyp residues (i.e., actual Pro-hydroxylation sites) in a protein can be determined by amino acid analysis of the protein, see Bergman, T., M. Carlquist, and H. Jomvall; Amino Acid Analysis by High Performance Liquid Chromatography of Phenylthiocarbamyl Derivatives. Ed. B. Wittmann-Liebold. Berlin: Springer Verlag, 1986. 45-55.

If one also knows the relative abundance of each Hyp-glycoside, the number of each Hyp species in a protein can be calculated. For instance, if a 200 residue protein contains 10 mol % Hyp, the 200-residue protein has 20 Hyp residues in it. If it also has 10% of its Hyp residues occurring as Hyp-arabinogalactan polysaccharide, 20% with Hyp-Ara₃ and 70% non-glycosylated Hyp, the protein contains 2 Hyp-arabinogalactan polysaccharides, 4 Hyp-Ara₃ moieties, and 14 non-glycosylated Hyp residues.

In this manner, one can determine the total number of actual Hyp-glycosylation sites.

### Experimental Determination of the Location of the Actual Proline-Hydroxylation Sites

The location of the hydroxyprolines (actual proline-hydroxylation sites) may be determined by fragmenting the proteins into peptides of sequenceable length, optionally deglycosylating the peptides, and then sequencing the peptides.

The proteins may be fragmented by treatment with one or more proteolytic non-enzymatic chemicals (e.g., cyanogen bromide) and/or one or more proteolytic enzymes.

Peptides may be deglycosylated, to simplify sequencing, by treatment with anhydrous hydrogen fluoride for 3 h at room temperature, according to the method of Moor and Lamport.

Peptides may be sequenced by automated Edman degradation. In each cycle, the liberated amino acid is analyzed by reverse phase HPLC, by which it is compared to amino acid standards. Hydroxyproline standards are available.

Alternatively, peptides may be sequenced by tandem mass spectrometry.

### Experimental Determination of the Location of the Actual Hyp-Glycosylation Sites

The first Hyp-glycosylation site identification for an HRGP was described in Kieliszewski, M., O'Neill, M., Leykam, J. F., and Orlando, R. “Tandem mass spectrometry and structural elucidation of glycopeptides from a hydroxyproline-rich plant cell wall glycoprotein indicate that contiguous hydroxyproline residues are the major sites of hydroxyproline O-arabinosylation,” Journal of Biological Chemistry, 270: 2541-2549 (1995). We used tandem mass spectrometry with collisionally induced dissociation to identify the arabinosylation sites in small glycopeptides isolated from a Douglas fir proline-rich protein (PRP).

Nonetheless, in general, it is difficult to determine the location (as distinct from the total number) of actual Hyp-glycosylation sites. Edman degradation is not likely to identify glycosylation sites unequivocally, and the structures are usually too complex for NMR structure analysis. MS/MS is primarily useful for very small glycopeptides with very small glycans. Hence, to proceed, one would normally fragment the glycoprotein into more readily analyzable fragments.

Unfortunately, a polypeptide with extensive Hyp glycosylation can be resistant to proteolysis, making it difficult to generate such fragments and thus to localize the actual Hyp-glycosylation sites.

In the context of the present invention, this is not an important limitation. In order to derive the rules for predicting whether a Hyp would be glycosylated, and how, we designed short peptides with simple sequence patterns containing prolines predicted to be hydroxylated, expressed them in plant cells, and determined which hydroxyprolines were glycosylated, and how.

If, on the other hand, we are attempting to determine whether a particular non-plant protein in fact has a native Hyp-glycomodule or (as a result of genetic engineering) or a substitution Hyp-glycomodule, we are usually primarily interested in the number of actual Hyp-glycosylation sites, rather than their location, because it is that number which affects whether we reach the threshold required for high-level secretion of the protein in plant cells.

Reaching that threshold is most in doubt when the number of predicted Hyp-glycosylation sites is small. But that also implies that the overall level of Hyp-glycosylation is likely to be low, and hence that the protein in question will not be resistant to proteolysis. In other words, the proteins which we are most likely to need to analyze to determine the location of the actual Hyp-glycosylation sites—e.g., so we can fine tune them by “fixing” predicted sites which were not actually glycosylated—are the ones which are most amenable to such analysis.

### Proteins of Interest

The proteins of interest may be known, naturally occurring proteins which, without further modification, already contain a sufficient number of Hyp-glycosylation sites to be desirably secreted if suitably expressed in plant cells. They may be referred to as predisposed proteins because they are predisposed, by virtue of their translated amino acid sequence, and its propensity to Pro-hydroxylation and Hyp-glycosylation, to the desired level of Hyp-glycosylation. (Of course, one may choose to increase that level still further.) The predisposed proteins may be non-plant proteins (preferably a vertebrate protein, more preferably a mammalian protein, most preferably a human protein), or they may be plant proteins which are not normally secreted.

The proteins of interest may also be known proteins which are modified, in accordance with the teachings of the present invention, in such manner as to increase the number of predicted or actual Hyp-glycosylation sites therein, to increase the likelihood of Hyp-glycosylation at an existing site, and/or to alter the nature of the glycosylation at a Hyp-glycosylation site. The modified (mutant) proteins may but need not feature additional mutations, for other purposes, as well.

Parental proteins for which such modification is considered desirable may be collectively referred to as Hyp-glycosylation-deficient proteins, and the suitably modified proteins as Hyp-glycosylation-supplemented proteins.

When such modification is considered desirable, it may be helpful to distinguish the parental protein from the expressed (modified) protein. While the latter is necessarily a mutant protein, the parental protein could be a naturally occurring protein, or a protein mutated for other purposes. In those embodiments in which the protein is not modified to affect Hyp-glycosylation, the expressed protein is also the parental protein.

While we speak formally of modifying a parental protein, it is not necessary to synthesize a parental protein and then modify it chemically. Rather, we mean that the parental protein is used as a guide in the design of a mutant protein which differs from it at one or more amino acid positions, so that the mutant protein can be formally characterized as a modification of the parental protein.

The plant cell-expressed and -secreted protein is preferably biologically active. However, if it is not itself biologically active, it preferably is cleavable, by a site-specific cleaving agent such as an enzyme, so as to release a biologically active polypeptide. If it is biologically active, it preferably retains one or more biological activities, and more preferably all biological activities, of the parental protein.

The parental protein which is mutated may be a non-plant protein (preferably a vertebrate protein, more preferably a mammalian protein, most preferably a human protein), or it may be a plant protein, as not all plant proteins are in fact predisposed to Hyp-glycosylation. (they may lack prolines, or the prolines may have a low predicted Hyp-score).

Most of the proteins of interest are proteins which comprise at least one predicted Hyp-glycosylation site, and which, if expressed and secreted in plant cells, exhibit Hyp-glycosylation (thus necessarily comprising at least one actual Hyp-glycosylation site, regardless of whether the location of the site is correctly predicted). Preferably, at least one predicted Hyp-glycosylation site is also an actual Hyp-glycosylation site.

However, a protein is also of interest if it is a non-plant protein which, in nascent form, comprises at least one proline, and exhibits Hyp-glycosylation, regardless of whether it was predicted to contain a Hyp-glycosylation sites. It is possible to simply express DNA encoding a non-plant protein, said DNA including at least one proline codon, and determine experimentally whether the protein, when expressed and secreted in plant cells, exhibits Hyp-glycosylation, without making any attempt to predict whether such Hyp-glycosylation would occur.

The mutant proteins of interest preferably have a greater number of actual Hyp-glycosylation sites and/or a greater number of predicted Hyp-glycosylation sites than does the parental protein.

Applicants are aware that certain proteins have previously been expressed and secreted in plant cells, which, by applicants' methods, are predicted to contain Hyp-glycosylation sites. The parties involved didn't recognize that there was any correlation between Hyp-glycosylation and the level of secretion, and hence had no motivation to generally express Hyp-glycomodule-containing proteins in plant cells, or to modify proteins to introduce or strengthen Hyp-glycomodules. Nonetheless, it may be desirable to disclaim the prior protein/plant cell combinations from the claimed methods, or the prior mutant proteins from the claimed mutant proteins, in order to avoid inadvertent anticipation. It should be understood that for the purpose of these disclaimers, and related preferred embodiments discussed in this section, the proteins are compared on the basis of the mature (non-signal) portions of their translated amino acid sequences, i.e., ignoring subsequent hydroxylation and glycosylation.

For the purpose of claims to methods of expressing and secreting proteins in plant cells, said protein being one which is not secreted by plant cells in nature, Applicants hereby disclaim certain protein-plant cell combinations, i.e., the expression and secretion in plant cells of particular species, of the particular Hyp-glycomodule-containing proteins (whether or not naturally occurring) which have previously been expressed and secreted in such cells, provided that such expression and secretion is within the body of prior art against this application.)

This disclaimer expressly includes, but is not limited to, the expression in tobacco cells of chimeric L6 single chain antibody (sFv and cys sFv), or of the anti-TAC sFv of Russell, U.S. Pat. No. 6,080,560, the thermostable Endo-1,4-beta-D-glucanase of Ziegler et al. (2000) (sequence database #P54583), the synthetic test proteins described by Shpak et al. (1999, 2001) and the mutant proteins described by Shimizu et al.

The synthetic test proteins of Shpak et al. (1999) were (Ser-Hyp)32-EGFP (a fusion of (Ser-Hyp)32, SEQ ID NO: 65, to enhanced green fluorescent protein, and (GAGP)3-EGFP (a fusion of (GAGP)3, SEQ ID NO:66, to enhanced green fluorescent protein.). The synthetic test proteins of Shpak et al. (2001) were fusions of (SPP)24 (SEQ ID NO:67), (SPPP)15 (SEQ ID NO:68) or (SPPPP)18 (SEQ ID NO:69) to enhanced green fluorescent protein.

The test proteins of Shimizu et al. were mutants of sweet potato sporamin, namely, the deletion mutants deltaPro, delta23-26, delta27-30, delta31-34, delta35-38, the substitution mutant P36Q, and, in the delta25-30 background, single substitution mutants in which one of residues 31-35 or 37-41 was replaced with another amino acid. Shimizu et al. didn't comment on the level of secretion in plant cells. It should be noted that for the sake of simplicity we have disclaimed almost all of Shimizu's test proteins without actually analyzing whether they have, or should have, Hyp-glycosylation modules. (The mutants in which P36 is replaced or deleted, i.e., deltaPro, delta 35-38 and P36Q, needn't be disclaimed because they necessarily lack a Hyp-glycosylation site.)

This disclaimer also expressly includes the protein-plant cell combinations set forth in Table Q below. It should be noted that a significant number of the proteins in this table are ones which lack predicted Hyp-glycosylation sites, and hence may be excluded by the main limitations of the claim. However, since these proteins do contain proline, they too are included in the disclaimer, just in case there is some actual Hyp-glycosylation site overlooked by the predictive method. Note that the recombinant human granulocyte-macrophage colony stimulating factor of Shin et al. (2003) (sequence database #AAU21240), and the human IgA1 of Karnoup, et al., are included in Table Q.

It must be emphasized that these publications didn't report a connection between the presence of a Hyp-glycomodule, and the level of secretion.

In a preferred embodiment, the method is one in which, if the protein is included in the above disclaimer of protein-plant cell combinations, the plant cell not only is not of the disclaimed plant species, it is not of any plant species belonging to the same family of plants, e.g., if the disclaimed prior expression was of the protein in tobacco cells, the protein is preferably not expressed in any Solanaceae plant cell.

In a more preferred embodiment, the method is one in which, the protein of interest is not any protein included in the above disclaimer of protein-plant cell combinations, regardless of the choice of plant cell. It must be emphasized that such disclaimer, and such preferred embodiment, don't exclude the use of a protein whose translated sequence differs from that of the protein of the prior art.

For the purpose of claims to non-naturally occurring proteins per se, Applicants hereby disclaim proteins which are non-naturally occurring, which comprise at least one Hyp-glycosylation module, and which are within the body of prior art against this application. This disclaimer expressly includes, but is not limited to, the chimeric L6 single chain antibody (sFv and cys sFv) and the antiTAC sFv of Russell, U.S. Pat. No. 6,080,560, the above-noted proteins described by Shimizu et al. and by Shpak et al. (1999, 2001), and the proteins whose names are italicized in Table Q. The Ziegler, Shin and Karnoup proteins noted above are naturally occurring proteins and hence are excluded by a non-naturally occurring” claim limitation, without the need for a particular disclaimer.

It will be appreciated that these disclaimers do not extend to mutants of the aforementioned disclaimed proteins, especially mutants which differ from the disclaimed proteins by one or more insertions or deletions, or by one or more non-conservative substitutions. However, the preferred proteins of the present invention are those which are less than 95% identical to the disclaimed proteins (or the proteins of the method claims' disclaimed protein-plant cell combinations), more preferably less than 80% identical, still more preferably less than 50% identical, and most preferably are not even homologous to the aforementioned disclaimed proteins (that is, the best alignment doesn't provide an alignment score which is significantly higher than what would be expected on the basis of amino acid composition).

One of the proteins listed in Tables P and Q is human collagen alpha1 type 1. In a preferred embodiment, the protein of the claimed proteins and methods is not a collagen of any human type, more preferably not a collagen of any type of any species, and still more preferably, is not a polypeptide consisting essentially of tandem repeats of the collagen helix motif GPP (or hydroxylated/glycosylated forms thereof). In one series of embodiments, the protein is a polypeptide which comprises an immunoglobin domain. Such polypeptides include immunoglobulin light chains, immunoglobulin heavy chains, single chain Fv (resulting from the fusion of the variable domains of the light and heavy chains, with or without an intermediate linker), and isolated immunoglobulin variable or constant domains. The polypeptides may be chimeric, e.g., combination of a variable domain from one species and a constant domain from another.

In another, more preferred series of embodiments, the protein of the claimed proteins and methods is not a polypeptide which comprises an immunoglobulin domain.

### Classification of Proteins

The proteins of interest (Hyp-glycosylation-predisposed proteins, the Hyp-glycosylation-deficient parental proteins, and the Hyp-glycosylation-supplemented proteins), may each be classified in a number of ways.

First, they may be classified according to sequence features. One important feature is the number of prolines in the translated sequence (i.e., ignoring possible subsequent hydroxylation and Hyp-glycosylation).

For the Hyp-glycosylation-deficient parental proteins, there may be zero, one, two, three, four, five, six, seven, eight, nine, ten or even more prolines. Typically, these Hyp-glycosylation deficient proteins have relatively few prolines, because each proline, if in a region favorable to hydroxylation and glycosylation, can become a Hyp-glycosylation site. The Hyp-glycosylation-predisposed proteins and Hyp-glycosylation supplemented proteins necessarily include at least one proline. They may have one, two, three, four, five, six, seven, eight, nine, ten or even more prolines, such as at least fifteen, at least twenty, or at least twenty five prolines.

In a related manner, they may be classified according to the percentage of amino acids which are prolines. In vertebrate proteins, on average, 5% of all of the amino acids are prolines. Hence, we may classify the Hyp-glycosylation-disposed and Hyp-glycosylation-deficient proteins as follows: less than 2.5% proline, 2.5-10% proline, and more than 10% proline.

Again, these proteins of interest may be classified according to the number of predicted Hyp-glycosylation sites. There may be zero (for Hyp-glycosylation-deficient proteins only), one, two, three, four, five, six, seven, eight, nine, ten or even more such sites, such at least fifteen, at least twenty, or at least twenty five such sites.

The proteins of interest may also be classified according to their total Hyp score, according to the quantitative standard method, for all of the prolines in the protein, divided by the score threshold. This could be, e.g., less than 2, at least 2 but less than 4, at least 4 but less than 8, at least 8 but less than 16, or at least 16.

Another structural feature of interest is the length of the protein. For this purpose, it is convenient to classify the proteins of interest into the following size classes: less than 35 amino acids, 35-69 amino acids, 70-139 amino acids, 140-279 amino acids, and 280 or more amino acids.

Still another structure feature of interest is the number of disulfide bonds, which can be zero, one, two, three, four or more than four.

A different approach to classification is one which considers the origin of the proteins. NCBI/GenBank maintains a taxonomy database. The proteins of interest may be classified according to their species of origin, each taxonomic grouping defining a particular class of proteins of interest. (Mutant proteins are classified according to the species of origin of the parental protein.) At the highest level, these are Archaea, Bacteria, Eukaryota, Viroids, Viruses, and Other. Eukaryotic taxons of particular interest include Viridiplantae and Vertebrata; within Vertebrata, Mammalia; and within Mammalia, Homo sapiens.

The protein may be a plant protein, in which case the plant may be an algae (which are in some cases also microorganisms), or a vascular plant, especially a gymnosperm (particularly conifers) or an angiosperm. Angiosperms may be monocots or dicots. The plants of greatest interest are rice, wheat, corn, alfalfa, soybeans, potatoes, peanuts, tomatoes, melons, apples, pears, plums, pineapples, fir, spruce, pine, cedar, and oak.

The protein may be that of a microorganism, in which case the microorganism may be an alga, bacterium, fungus or virus. The microorganism may be a human or other animal or plant pathogen, or it may be nonpathogenic. It may be a soil or water organism, or one which normally lives inside other living things, or one which lives in some other environment.

The protein may be that of an animal, and the animal may be a vertebrate or a nonvertebrate animal. Nonvertebrate animals which are human or economic animal pathogens or parasites are of particular interest. Nonvertebrate animals of interest include worms, mollusks, and arthropods.

The vertebrate animal may be a mammal, bird, reptile, fish or amphibian. Among mammals, the animal preferably belongs to the order Primata (humans, apes and monkeys), Artiodactyla (e.g., cows, pigs, sheep, goats, horses), Rodenta (e.g., mice, rats) Lagomorpha (e.g., rabbits, hares), or Carnivora (e.g., cats, dogs). Among birds, the animals are preferably of the orders Anseriformes (e.g., ducks, geese, swans) or Galliformes (e.g., quails, grouse, pheasants, turkeys and chickens). Among fish, the animal is preferably of the order Clupeiformes (e.g., sardines, shad, anchovies, whitefish, salmon).

A third approach to classification is by gene ontology, and is discussed in a later section.

If any defined class of proteins, or any combination of defined classes of proteins, is inherently anticipated by a prior art protein, it is within the contemplation of the inventors to exclude it from the claims, while otherwise retaining generic coverage.

### Specific Proteins

The proteins of interest (without differentiation between predisposed proteins and parental proteins) include, but are not limited to, (1) the specific proteins set forth in sections I-III, classifying proteins on the basis of their native predicted Hyp-glycosylation sites, and (2) whether or not already listed under (1), vertebrate, preferably mammalian, more preferably human, proteins selected from the group consisting of growth hormone, growth hormone mutants which act as growth hormone or prolactin agonists or antagonists (a category discussed in more detail below), growth hormone releasing hormone, somatostatin, ghrelin, leptin, prolactin, prolactin mutants which act as prolactin or growth hormone antagonists, monocyte chemoattractant protein-1, interleukin-10, pleiotropin, interleukin-7, interleukin-8, interferon omega, interferon-Alpha 2a and 2b, interferon gamma, interleukin-1, fibroblast growth factor 6, IFG-1, insulin-like growth factor I, insulin, erythropoietin, and GMCSF, and any humanized monoclonal antibody or monoclonal antibody, all except as explicitly disclaimed above.

### Level of Expression

The level of expression of a protein may be determined by any art-recognized method. The level of expression is directly related to the level of transcription, which can be determined by a northern blot analysis of the corresponding mRNA. The level of expression may also be determined by Western blot analysis. (If the Western blot analysis is of the protein in the culture medium, then the analysis is measuring the level of protein both expressed and secreted. To determine the total expression, the cells may be lysed and the analysis consider the lysate as well as the medium.)

### Level of Secretion

Preferably, the non-plant proteins of the present invention are secreted in plant cells at a level which is increased relative to the level at which they have previously been secreted in non-plant cells.

Preferably, the modified proteins of the present invention are secreted in plant cells at a level which is increased relative to that at which the parental protein can be secreted, using the identical plant cell species, culture conditions, promoter and secretion signal.

The level of secretion may be determined by any art-recognized method, including Western blot analysis of the level of the protein in the culture medium.

The level of secretion may be characterized by the concentration of the protein in the medium, by the level of the protein in the medium as a percentage of total soluble protein TSP) in the medium, or by the level of the protein in the medium as a percentage of total secreted proteins in the medium.

Preferred (high) levels of secretion are at least 1 mg/L protein equivalent in medium, more preferably at least 5 mg/L, still more preferably at least 10 mg/L to 150 mg/L, most preferably at least about 30 mg/L. It is expected that for the parental proteins lacking Hyp-glycosylation, the level of secretion is typically less than 100 ug/L, or even less than 1 ug/L. That implies preferred, increases in secretion of at least 10 fold, more preferably at least 100 fold, still more preferably at least 1,000-fold, most preferably at least 10,000-fold.

With addition glycomodules, we found that secretion of human IFN alpha-2 was improved from 0.2-0.4% TSP (0.002-0.02 mg/L in medium) for the native protein to 0.9-1.5% TSP (7-11 mg/L for one with an (SO)2 glycomodule (amino acids 1-4 of SEQ ID NO:118), 2.0-3.5% TSP (17-28 mg/L) for one with an (SO)10 (amino acids 1-20 of SEQ ID NO:118) addition glycomodule, and 2.4-3.0% TSP (23-27 mg/L) for one with an (SO)20 (SEQ ID NO:118) addition glycomodule. Likewise, for human growth hormone, secretion was improved from 0.3-0.6% TSP (0.001-0.07 mg/L) for the native protein to 2.2-4.0% TSP (16-35 mg/L) for HGH with the aforementioned (SO)10 addition glycomodule.

Preferably, the protein of the present invention, as a result of the native or introduced Hyp-glycomodules, the choice of secretion signal peptide, and, optionally, N-glycosylation, has a level of secretion of at least 1% TSP, more preferably at least 2% TSP.

Preferably, the secreted protein of interest is at least 50%, more preferably at least 75%, still more preferably at least 85%, of the secreted proteins in the medium.

### Non-Naturally Occurring Mutant Proteins

### Relationship of Mutated Protein to Parental Protein

A “non-naturally occurring protein” is one which is not known to occur in a cell or virus, except as a result of human manipulation.

The present invention contemplates mutation of a parental protein to create a mutant, non-naturally occurring protein with an increased propensity to Pro-hydroxylation and/or Hyp-glycosylation. Preferably there is a net increase in the number of Pro-hydroxylation and Hyp-glycosylation site. More preferably, no Pro-hydroxylation and Hyp-glycosylation sites are lost as a result of the mutation.

The practitioner designing the mutant protein will of course have a particular parental protein in mind. In general, the mutant is designed with reference to a particular protein, i.e., incorporating predetermined insertions, deletions and substitutions relative to a predetermined parental protein. However, if there are a sufficient number of mutations, the mutant may come to more closely resemble some other protein, either fortuitously, or because the practitioner was guided by more than one parental protein in designing the mutant protein.

A first protein may be considered a mutant of a second protein if the first protein has an amino acid sequence which, when aligned by BlastP, with default parameters, to the sequence of the second protein, generates an alignment score which is statistically significant, i.e., is a higher score then would be expected if the mutant amino acid sequence were aligned with randomly jumbled amino acid sequences of the same length and amino acid composition. Thus, even if the predetermined parental protein used in such design is not known to the practitioner, it may be identifiable by using the sequence of the mutant protein as a query sequence in searching a suitable sequence database containing the parental sequence. A mutant protein is not necessarily non-naturally occurring, as a mutant of protein A may coincidentally be identical to naturally occurring protein B.

A protein is considered to be a mutant of a non-plant protein if 1) it has known to have been designed as a mutant of a predetermined non-plant protein and remains more than 50% identical to that non-plant protein, 2) it was made by expression of a gene derived by mutation of a gene encoding a non-plant protein, 3) it has, or comprises a sequence which has, a biological activity which is found in a naturally occurring non-plant protein but which biological activity is not known to occur in any plant protein, or 4) it has, ignoring all Hyp-glycomodules as herein defined, a higher alignment score (aligning with BlastP, default settings) with respect to a non-plant protein than with respect to any known plant protein. The reason we ignore Hyp-glycomodules is that Hyp-glycomodules are common in some plant proteins and hence incorporating Hyp-glycomodules into, e.g., a human protein, will cause it to have a higher alignment score with those plant proteins than would otherwise be the case. If need be, each of these four definitional considerations may be used to define a separate class of mutants of non-plant proteins.

Mutants of vertebrate, mammalian and human proteins, as well as mutants of non-vertebrate, non-mammalian, and non-human proteins, may be defined in an analogous manner.

Mutations may take the form of insertions, deletions or substitutions. While we recognized that a substitution may be conceptualized as a deletion followed by an insertion, we don't so consider it here. When the sequence of the mutant protein is aligned to that of the parental protein, each residue of the mutant protein is 1) aligned with an identical residue of the parental protein (in which case that is considered an unmutated position), 2) aligned with a non-identical residue of the parental protein (in which case that is considered a substitution), or 3) aligned with a null character (usually represented as a space or hyphen), implying that there is no corresponding residue in the parental protein (in which case the residue in question is considered an inserted amino acid). A residue of the parental protein, instead of being aligned with a residue of the mutant protein (resulting in the position being considered either unmutated or substituted), may be aligned with a null character, implying that there is no corresponding residue in the mutant protein (in which case the residue in question is considered a deleted amino acid).

### Percentage Identity and Percentage Similarity

When the mutated protein differs from the parental protein by the creation of a substitution Hyp-glycomodule, the protein can retain a high degree of sequence identity to the parental protein. For example, it may be possible to create a new predicted Hyp-glycosylation site by as little a single substitution mutation. In the worst possible case, a Hyp-glycosylation site can be created by five consecutive substitution mutations. Plainly, one can also have the intermediate situation in which the new Hyp-glycosylation site is created by two, three or four mutations within a consecutive five amino acid subsequence of the parental protein.

Thus, if a protein is, say, two hundred amino acids in length (a typical length for a mammalian single domain protein), a single Hyp-glycosylation site can be created by just 1-5 substitution mutations, which corresponds to a change in percentage identity (see below) of just 0.5-2.5%. Likewise, two new Hyp-glycosylation sites can be created by just 1-10 substitution mutations (the “1” is not a typographical error; a single substitution affects the Hyp-scores of prolines up to two amino acids before it and up to two amino acids after it, and therefore could cause the Hyp-scores of two or more nearby prolines to exceed the preferred threshold of the prediction algorithm), corresponding to a change in percentage identity of just 0.5-5%. If no other mutations were made, the resulting modified protein would still be at least 95% identical to the parental protein.

Of course, mutation is not limited to proteins of two hundred amino acids length, and the number of additional Hyp-glycosylation sites is not limited to one or two. The practitioner must strike a balance between the addition of Hyp-glycosylation sites (with the potential for improved secretion and other advantages) and any adverse effect on biological activity and/or immunogenicity.

One method of concisely stating the relationship of two proteins is by stating a percentage identity. This application contemplates two percentage identities, primary and secondary. The primary percentage identity is determined by first aligning the two proteins by BlastP (a local alignment algorithm), with default parameters, and then expressing the number of matching aligned amino acids as a percentage of the length of the overlap region (which includes any gaps introduced during the alignment process).

The relationship of the proteins may also be expressed by a secondary (“global”) percentage identity calculation, in which the number of matches is expressed as a percentage of the length of the longer sequence (which is likely to be the mutant protein).

If the mutant protein results from simple addition of one or more Hyp-glycomodules to the amino or carboxy terminal of the parental protein, then the mutant protein remains identical to the parental protein in the overlap region, i.e., the calculated primary percentage identity is 100% even though the mutant protein is longer than the parental protein. However, the secondary percentage identity would be less than 100%. For example, the addition of (Ser-Hyp) 10 to a 200 amino acid protein would result in a secondary percentage identity of 200/220, or about 91%.

Preferably, the mutants of the present invention are at least 50% identical, more preferably at least 60%, at least 70%, at least 80%, at least 85%, or at least 90%, such as at least 91, 92, 93, 94, 95, 96, 97, 98, or 99% identical, to the parental protein when percentage identity is calculated by the primary and/or by the secondary method. To be considered a mutant, it cannot be identical to the parental protein, but as explained above, it may nonetheless have a primary percentage identity which is 100%.

In like manner, one may define a primary and secondary percentage similarity. Two amino acids are considered to be similar if, in the default scoring matrix for BlastP, their alignment is assigned a positive score.

### Conservative Substitution and Related Concepts

Substitutions can be conservative and/or nonconservative. In conservative amino acid substitutions, the substituted amino acid has similar structural and/or chemical properties with the corresponding amino acid in the reference sequence. By way of example, conservative substitutions (replacements) are defined as exchanges within the groups set forth below:

I small aliphatic, nonpolar or slightly polar residues—Ala, Ser, Thr (Pro, Gly)

II negatively charged residues and their amides Asn Asp Glu Gln

III positively charged residues—His Arg Lys

IV large aliphatic nonpolar residues—Met Leu Ile Val (Cys)

V large aromatic residues—Phe Tyr Trp

Three residues are parenthesized because of their special roles in protein architecture. Gly is the only residue without a side chain and therefore imparts flexibility to the chain. Pro has an unusual geometry which tightly constrains the chain. Cys can participate in disulfide bonds, which hold proteins into a particular folding. These residues sometimes exchange with the other members of their exchange group, and at other times are not replaceable.

In some cases, it is has been found that Cys, because of its size and polarity, can be safely replaced with Ser, Thr, Ala or Gly. Hence, this may also be considered a conservative substitution, but not the other way around.

The following exchanges are considered highly conservative: Glu/Asp, Arg/Lys/His, Met/Leu/Ile/Val, and Phe/Tyr/Trp.

Non-conservative substitutions may be further classified as semi-conservative or as strongly non-conservative. Inter-group exchanges of group I-III residues may be considered semi-conservative, as they are all hydrophilic, neutral (Gly), or only slightly hydrophobic (Ala). Inter-group exchanges of Group IV and IV residues can be considered semi-conservative, as they are all strongly hydrophobic. Exchanges of Ala with amino acids of groups II-V can be considered semi-conservative, as this is the principle underlying Ala scanning mutagenesis. All other non-conservative substitutions are considered strongly non-conservative.

Preferably, within each Hyp-glycomodule, all substitutions are at least semi-conservative, more preferably, at least conservative.

Preferably, outside each Hyp-glycomodule, all substitutions are at least semi-conservative, more preferably, at least conservative, and most preferably, are highly conservative.

### Miscellaneous Mutation Considerations

Preferably, if the parental protein is a member of a family of homologous proteins, each mutated position is one which is not a conserved position in the family.

The mutant protein may differ from the parental protein by further mutations not related to the control of the level of hydroxylation of proline and/or glycosylation of hydroxyproline, but it is desirable that such further mutations not substantially impair the biological activity of the protein (or, if the protein is to be further processed to yield the final biologically active molecule, of the latter).

### Hyp-Glycomodules

A protein comprising at least one Hyp-glycosylation site must necessarily comprise at least one Hyp-glycomodule. They may comprise, e.g., two, three, four, five, six or more Hyp-glycomodules. Each Hyp-glycomodule comprises, in accordance with the definition, at least one Hyp-glycosylation site. Again in accordance with the definition, Hyp-glycomodules may be adjacent to each other, or separated.

### Hyp-Glycomodules in Mutant Proteins

If a Hyp-glycomodule occurs in a mutant protein, it may be classified according to its relationship, if any, to the underlying mutations which differentiate that mutant protein from a parental protein. Thus, it may be an insertion Hyp-Glycomodule (which optionally may further include substitutions and/or deletions), a substitution Hyp-Glycomodule (which optionally may further include deletions, but cannot include insertions), a deletion Hyp-Glycomodule (wherein only one or more deletions differentiate it from the aligned parental sequence), or a native Hyp-Glycomodule (which is identical to an aligned Hyp-Glycomodule of the parental protein).

An insertion Hyp-glycomodule is characterized as the result, at least in part, of insertion of one or more amino acids at the amino terminal, the carboxy terminal, or internally between two pre-existing amino acid positions, of the parental protein. If the insertions are solely of one or more amino acids at the amino or carboxy terminals, it may be further characterized as an addition glycomodule (a subtype of insertion glycomodule).

An insertion Hyp-glycomodule may, but need not, further involve one or more substitutions (replacements) and/or one or more deletions (without replacement thereof) of additional amino acids of the parental protein. If it is solely the result of insertion, it may be characterized as a simple insertion (or addition) glycomodule.

### The Corresponding Segment of the Original Protein.

The present specification may refer to a Hyp-glycomodule as a substitution Hyp-glycomodule if it can be characterized as being solely the result of one or more substitutions (replacements), and, optionally one or more deletions, of amino acids of the parental protein. In other words, if the mutation of the parental protein to incorporate the glycomodule requires any insertions of amino acids, the glycomodule is an insertion glycomodule, not a substitution glycomodule. We are aware that a substitution can be thought of as the result of a deletion followed by an insertion at the same location. However, the insertions we have in mind are insertions in-between positions of the parental protein.

If the mutant protein is a Hyp-glycosylation-supplemented protein, then at least one of the Hyp-glycomodules must be an insertion, substitution, or deletion Hyp-Glycomodule. However, it may optionally include one or more native Hyp-Glycomodules.

In a naturally occurring protein, the Hyp-Glycomodule is necessarily a native Hyp-Glycomodule.

### Proline Skeletons

Hyp-glycomodules may be classified according to the nature of their proline skeleton, i.e., the locations of the prolines within the corresponding nascent Hyp-glycomodule.

In some embodiments, the Hyp-glycomodule has a regularly and uniformly spaced proline residue skeleton. For example, the Hyp-glycomodule may consist essentially of a series of contiguous proline residues. Alternatively, the Hyp-glycomodule may have a proline skeleton in which the proline residues are regularly and uniformly spaced, but non-contiguous, such as the proline skeleton patterns (Pro-X)n, (Pro-X-X)n, (Pro-X-X-X)n or (Pro-X-X-X-X)n, where n is at least two.

In other embodiments, the Hyp-glycomodule has a proline skeleton in which the prolines are regularly but not uniformly spaced, e.g., there is a repeating pattern of prolines such as (X-P-P-P)n or (X-P-P-X)n, where n is at least two.

In yet other embodiments, the Hyp-glycomodule has a proline skeleton in which the prolines are irregularly spaced.

The proline skeleton of the Hyp-glycomodule may be a combination of the above skeleton types or patterns, and may also include irregularly distributed prolines. It will be understood that in the formulae set forth above, the X may be different both within a single iteration of the repeating pattern, or from iteration to iteration. However, it is preferable that the X be the same amino acid.

### Hydroxyproline Skeletons

In a like manner, one may define the hydroxyproline skeleton of the mature Hyp-glycomodules.

### Classification by Glycosylation

Hyp-glycomodules may be classified according to the nature of their glycosylation. Thus, a Hyp-glycomodule as now defined may include only arabinogalactosylated Hyp-glycosylation sites (an arabinogalactan Hyp-glycomodule), only arabinosylated Hyp-glycosylation site (an arabinosylation Hyp-glycomodule), or a combination of the two (a mixed Hyp-glycosylation) Hyp-glycomodule. The nature of the proline skeleton has a direct effect on the nature of the glycosylation, as is evident from the glycosylation prediction methods set forth above. It is also possible that the Hyp may be glycosylated other than with arabinose or arabinogalactan, in which case the Hyp-glycomodule may be characterized as exotic.

### Preferred Arabinosylation Hyp-Glycomodules

For arabinosylation Hyp-glycomodules (where glycosylation sites are contiguous Hyp residues), genes tailored for expression preferably encode sequences comprising contiguous Pro residues, i.e., (Pro)n, where n=2-1000. The value of n may be at least 3, 4, 5, 6, 7, 8, 9, 10, 50, 100, or 500, and/or less than 999, 998, 997, 996, 995, 994, 993, 992, 991, 990, 900, 800, 700, 600, or 500; or indeed any other subrange of 2-1000 Most of the Pro residues in these sequences will be hydroxylated to hydroxyproline and subsequently O-glycosylated with arabinosides ranging in size from one to five arabinose residues.

If we reconsider these teachings in the light of the prediction algorithm, then it is apparent that if the number of consecutive prolines is five or more, then, for one or more “central” prolines, the positions −2, −1, +1 and +2 will all be proline, resulting in a matrix score of 11.

Also, as the number of consecutive prolines increases, so, too, will the local composition factor for the prolines. If the block is 21 or more consecutive prolines, then one or more central” prolines will have an LCF of 1 (the maximum possible value).

### Preferred Arabinogalactan Hyp-Glycomodules

For arabinogalactan Hyp-glycomodules (where the glycosylation sites are clustered non-contiguous Hyp residues), the genes may comprise sequences which encode variations of (Pro-X)n and (X-Pro)n, where n=1-11000, and X is Ser, Ala, Thr, Pro or Val. The value of n may be, e.g., at least 2, 3, 4, 5, 6, 7, 8, 9, 10, 50, 100, or 500, and/or less than 999, 998, 997, 996, 995, 994, 993, 992, 991, 990, 900, 800, 700, 600, or 500, or indeed any other subrange of 1-1000. Many of the Pro residues in these sequences will be hydroxylated to hydroxyproline (Hyp) and subsequently O-glycosylated with arabinogalactan oligosaccharides or polysaccharides.

In the light of the standard prediction method, with the quantitative standard method used to predict Pro-hydroxylation, we can see that a repeating sequence of the form X-Pro or Pro-X (where X is Lys, Ser, Thr, Val, Gly, or Ala) will, if there are sufficient repetitions, establish that most of the target prolines have Ser, Thr, Val, Gly, or Ala in the −1 and +1 positions, and Pro in the −2 and +2 positions. The matrix scores will vary depending on the choice of X in each repetition. If X is the same amino acid for all of the repetitions, then the matrix score for all prolines other than the first and last one in the repeat sequence will be, for X=Ser or Ala, +11; for X=Thr, +8; for X=Val, +7; and for X=Gly, 3.25.

Hence, it would appear that the order of preference of repeat X-Pro sequences would be Ser-Pro, Ala-Pro>Thr-Pro>Val-Pro>Gly-Pro, and there is an analogous order of preference for Pro-X repeats. It should be appreciated that, as the number of repetitions increases, the distinction between (X-Pro)n and (Pro-X)n diminishes, as it is apparent only at the ends of the repeat region.

If X is the same for all repeats in a block of consecutive dipeptide repeats, then, once the number of repetitions exceeds ten, one or “central” prolines will have a local composition factor such that 11/21 amino acids in the preferred 21 amino acid window are proline and 10/21 are the alternative amino acid, yielding an absolute entropy of 0.998364, a relative entropy of 0.231, and a relative order (local composition factor) of 0.769 (which, being greater than the preferred baseline of 0.4, means that the local composition factor is favorable). While use of the same X for all repeats is preferred, it is not required. Preferably, the X's for each repeat are chosen so that the average local composition factor score for all of the Pro's in the Hyp-glycomodule is at least equal to the baseline, which has a preferred value of 0.4.

### Number of Hyp-Glycomodules

The proteins of the present invention feature at least one predicted/actual Hyp-glycomodule. This may be an insertion Hyp-glycomodule (preferably an addition Hyp-glycomodule, more preferably a simple addition Hyp-glycomodule) or a substitution Hyp-glycomodule. If there is more than one Hyp-glycomodule, they may be of the same or different types.

### Design of Insertion Hyp-Glycomodules

The design of insertion Hyp-glycomodules is discussed in detail in the prior applications, and the preferred arabinogalactosylation and arabinosylation Hyp-glycomodules set forth above are preferred insertion Hyp-glycomodules.

An insertion Hyp-glycomodule is preferably added at the amino-terminal and/or the carboxy terminal of the biologically active protein. The glycomodule may be joined directly to the terminal amino acid of the parental protein, or indirectly. In the latter case, the Hyp-glycomodule is linked to the native human protein moiety by a spacer which either 1) acts to distance the native human protein moiety from the Hyp-glycomodule in such manner as to increase the retention of native human protein biological activity by the Hyp-glycomodule-spacer-human protein fusion relative to that retained by a direct Hyp-glycomodule-human protein fusion, or 2) provides a site-specific cleavage site for an enzyme or chemical agent such that, after cleavage at that site, a new product is generated which does have the desired biological activity.

Spacers suitable for distancing are discussed in, e.g., Hoffman, U.S. Pat. No. 6,124,114, “Hemoglobins with intersubunit disulfide bonds”; U.S. Pat. No. 6,828,125, “DNA encoding fused di-alpha globins and use thereof”; U.S. Pat. No. 5,844,089, “Genetically fused globin-like polypeptides having hemoglobin-like activity”; U.S. Pat. No. 5,844,088 Hemoglobin-like protein comprising genetically fused globin-like polypeptides; U.S. Pat. No. 5,776,890 Hemoglobins with intersubunit disulfide bonds; U.S. Pat. No. 5,744,329, “DNA encoding fused di-beta globins and production of pseudotetrameric hemoglobin”; U.S. Pat. No. 5,545,727, “DNA encoding fused di-alpha globins and production of pseudotetrameric hemoglobin”. It may also be helpful to consult a loop library, see e.g., http://chem250a.chem.temple.edu/guide.htm

Site-specific cleavage sites are discussed in, e.g., Walker, “Cleavage Sites in Expression and Purification,” http://stevens.scripps.edu/webpage/htsb/cleavage.html; Barrett, et al., The Handbook of Proteolytic Enzymes. Please note that site-specific cleavage need not be achieved enzymatically; consider, e.g., the action of cyanogen bromide. In general, it is preferable to use cleavage agents which are specific for a cleavage site which is longer than two amino acids, so as to reduce the possibility that the parental protein will include a site sensitive to the desired agent. The cleavable linker and cleavage agent are chosen so that the biologically active moiety of the fusion protein is not cleaved, only the linker connecting that moiety to the insertion (addition) glycomodule.

Alternatively, a Hyp-glycomodule may be inserted in the interior of the parental protein. If so, then if the protein is a multi-domain protein, it is preferably inserted at an inter-domain boundary. Other possible preferred insertion sites include turns and loops, or sites known, by comparison with homologous proteins, to be tolerant of insertion.

If an X-Ray structure is available, one may look at the B-factors (temperature factors) for the atoms in the vicinity of the proposed insertion. B-factors are indicative of the precision of the atom positions. If the model is of high quality (e.g., an R factor of 2 or less in a model with a resolution of 2.5 angstroms or better), then a high B-factor is likely to be indicative of freedom of movement of the atoms in that region. Preferably, the B-factor is at least 20, more preferably, at least 60. Similar considerations apply to NMR structures.

An addition Hyp-glycomodule may replace a portion of the amino-terminal or carboxy terminal of the biologically active protein, provided that it still extends beyond that original terminal. (If the glycomodule merely replaces a amino or carboxy terminal portion with a sequence of the same or lesser length, it is denoted a substitution glycomodule.)

One or more deletions may also be advantageous. For example, in the case of membrane-spanning or -anchored enzymes, it may be advantageous to delete the membrane-spanning or -anchoring domain (avoiding the intrinsic tendency of glycosyltransferases, for example, to associate with ER/Golgi membranes).

A Hyp-glycomodule may replace a sequence of the parental protein. If a Hyp-glycomodule replaces a portion of the protein, then the non-proline residues of the Hyp-glycomodule may be chosen to minimize the number of substitutions, or at least the number of non-conservative substitutions, by which the replacement Hyp-glycomodule differs from

### Design of Substitution Hyp-Glycomodules

If a protein of interest is completely lacking in Hyp-glycosylation sites, or if the practitioner would prefer to increase the number of Hyp-glycosylation sites, there are, as previously stated, three basic strategies: add at least one glycomodule to the amino or carboxy terminal, insert the glycomodule into the internal sequence of the protein, or create Hyp-glycosylation sites by one or more substitutions, thereby creating glycomodules within the original length of the protein.

There are essentially two considerations governing such substitutions: 1) the effect on the probability of Hyp-glycosylation at or near the substitution site, and 2) the effect of the substitution on biological activity.

In general, the substitutions will take the form of 1) replacement of non-proline residues with prolines so as to create new sites, and/or 2) replacement of non-proline residues which are near (especially within two amino acids of) a proline so as to render that proline more likely to experience hydroxylation and glycosylation.

Information about the wild-type protein may be useful in identifying where the substitutions might be tolerated. Such information could include any of the following:
a 3D structure for the protein or a homologous protein (changes are more likely to be tolerated if they are at the surface and are distal to the known binding sites of the protein)
the binding sites of the protein (this is typically determined either by testing fragments for activity or by some systematic mutagenesis method)
alignment of the sequence of the protein with that of homologous proteins (proteins with similar sequences and biological activities) and identification of the positions at which there is amino acid variability (the greater the variability, the more likely it is that such position will be tolerant of mutation)
homologue-scanning mutagenesis or alanine-scanning mutagenesis studies of the protein or of a homologous protein
secondary structure predictions for the protein (a mutation is more likely to be tolerated in a loop than in an alpha helix. A mutation in an alpha helix is more likely to be tolerated if the replacement amino acid has a strong alpha helical propensity.)

One may also take into account whether the proposed replacement amino acid is one generally considered to be a “conservative substitution”, or at least a “semi-conservative substitution”, for the original amino acid.

Taking into account both the conservative and semi-conservative substitution definitions and the table of matrix values, it can be seen that the following substitutions are likely to be of benefit:

replacement of other group IV residues with Val

replacement of Cys with Ser, Thr, Ala or, less attractively, Gly

replacement of −1 position Asp, Asn or Gln with Glu

If a protein comprises one or more prolines with a low Hyp-score, it is preferable to modify the nearby non-proline residues to increase that score, rather than to introduce altogether new prolines into the sequence. This is because of the unique effect of proline upon secondary structure (it tends to introduce rigidity into the polypeptide chain). However, introduction of proline is not excluded. The introduction of proline is likely to be more tolerated in a position outside an alpha helix than in an alpha helix. In an alpha helix, it is more likely to be tolerated within the first turn.

### Design of Deletion Hyp-Glycomodules

Deletions may be made at the amino or carboxy terminal (also called truncation), and/or internally. Internal deletions are preferably made in the same protein regions which are the preferred locations for internal insertions. Deletions are most likely to be made to bring together two prolines, or a proline and one of the favored flanking amino acids (Ser, Tbr, Val, Ala), or to eliminate an unfavorable amino acid (especially those with longer range effects, such as Cys, Tyr, Lys and His). However, as a practical matter, deletions are more likely to adversely affect biological activity than are substitutions or additions, and deletions can only make an existing Pro more favorable to hydroxylation and glycosylation, they don't increase the number of Pro in the protein.

The teachings of this section apply, mutatis mutandis, to the consideration of deletions in insertion Hyp-glycomodules or substitution Hyp-glycomodules.

### Effect of Disulfide Bonding

Protein domains with disulfide bonds might not exhibit Pro hydroxylation or Hyp glycosylation, even at residues predicted to be favorable sites, as the disulfide bonds hold the protein in a folded conformation which hinders presentation of the polypeptide to the co- and/or post-translational machinery involved in hydroxylation of proline and/or glycosylation of hydroxyproline. Hence, it is preferable that the protein to be expressed not comprise any cysteines expected to participate in disulfide bonds.

The art teaches that disulfide bond formation can be avoided or reduced by eliminating cysteines not essential to biological activity, e.g., by replacing the cysteines with serine, threonine, alanine or glycine.

If one or more disulfide bonds must be maintained, then it may be desirable to use a larger number of predicted Hyp-glycosylation sites and/or distribute the predicted Hyp-glycosylation sites throughout the molecule so as to maximize the chance that at least one site is in fact glycosylated despite the folded conformation.

It is also possible to use a variety of experimental methods to identify regions which are exposed, despite the folded conformation. For example, one may expose the folded protein to a chemical protein surface labeling agent and then determine which residues have been chemically modified by that agent. An agent of particular interest is tritium, as it is possible to elicit tritium exchange with all exposed hydrogens.

Of course, if the 3D-structure of the protein has been determined by X-ray diffraction or by NMR, this may be used to identify surface sites for modification.

### Proline Substitutions

Proline substitutions have been used to increase thermostability. See e.g., Allen, “Stabilization of Aspergillus awamori glucoamylase by proline substitution and combining stabilizing mutations,” Protein Eng. 11: 783-8 (1998); Muslin, et al., “The effect of proline insertions [sic] on the thermostability of a barley alpha-glucosidase,” Protein Eng. 15(1): 29-33 (2002). They have also been used to alter enzyme selectivity. Liu, et al., “Mutations to alter Aspergillus awamori glucoamylase selectivity . . . ”, Protein Eng. 12(2): 163-172 (1999). See also Watanabe, “Analysis of the critical sites for protein thermostabilization by proline substitution in oligo-1,6-glucosidase, etc.”, Appl. Environ. Microbiol. 62(6): 2066-73 (1996).

Proline scanning mutagenesis (systematic synthesis of a series of single proline substitution mutants, usually corresponding to the non-proline positions in a contiguous region of a protein) is described in Schulman and Kim, “Proline scanning mutagenesis of a molten globule reveals non-cooperative formation of a protein's overall topology,” Nat. Struct. Biol., 3:682-7 (1996), Orzaez, et al., “Influence of proline residues in transmembrane helix packing,” J. Mol. Biol., 335(2): 631-40 (2004), Sugase, et al., “Structure-activity relationships for mini atrial natriuretic peptide by proline-scanning mutagenesis and shortening of the peptide backbone,” Bioorg Med Chem Lett 12(9): 1245-7 (2002).

According to Suckow, et al., “Genetic Studies of the Lac Repressor XV: 4000 Single Amino Acid Substitutions and Analysis of the Resulting Phenotypes on the Basis of the Protein Structure,” J. Mol. Biol. 261: 509-23 (1996), despite proline's ability to distort local second structure, replacement of the native Lac Repressor amino acid with proline resulted in a nonfunctional (1-) phenotype in only “64 of 154 (=42%) of all amino acid positions in alpha-helices, 27 of 57 (=47%) of all amino acids positioned in beta-sheets and 21 of 117 (=18%) of all amino acids in loops and turns . . . .” Moreover, “the positions where a replacement by proline results in an I-phenotype are clustered and not uniformly spread across the secondary structure elements of the protein ([Suckow] FIG. 4). Most secondary structure elements where no specific function of the protein is located, alpha-helices as well as beta-sheets or turns, seem to tolerate a proline insertion.”

### Growth Hormone Superfamily Mutants

Growth hormone, prolactin and placental lactogen mutants are of interest. A mutant may be characterized as a growth hormone mutant if, after alignments by BlastP, it has a higher percentage identity with a vertebrate growth hormone than it does with any known vertebrate prolactin or placental lactogen. Prolactin and placental lactogen mutants are analogously defined.

This mutant may be an agonist, that is, it possesses at least one biological activity of a vertebrate growth hormone, prolactin, or placental lactogen. It should be noted that a growth hormone may be modified to become a better prolactin or placental lactogen agonist, and vice versa. The mutant may be characterized as a growth hormone mutant if, after alignments by BlastP, it has a higher percentage identity with a vertebrate growth hormone than it does with any known vertebrate prolactin or placental lactogen. Prolactin and placental lactogen mutants are analogously defined.

Alternatively, the mutant may be an antagonist of a vertebrate growth hormone, prolactin, or placental lactogen. In general, the contemplated antagonist is a receptor antagonist, that is, a molecule that binds to the receptor but which substantially fails to activate it, thereby antagonizing receptor activity via the mechanism of competitive inhibition. The first identification of GH mutants that encoded biologically active GH receptor antagonists was in Kopchick et al., U.S. Pat. Nos. 5,350,836, 5,681,809, 5,958,879, 6,583,115, and 6,787,336, and in Chen et al., 1991, “Functional antagonism between endogenous mouse growth hormone (GH) and a GH analog results in dwarf transgenic mice”, Endocrinology 129:1402-1408, Chen et al., 1991, “Glycine 119 of bovine growth hormone is critical for growth promoting activity” Mol. Endocrinology. 5:1845-1852, and Chen et al., 1991, “Mutations in the third .alpha.-helix of bovine growth hormone dramatically affect its intracellular distribution in vitro and growth enhancement in transgenic mice”, J. Biol. Chem. 266:2252-2258. All of these references (hereinafter, “Kopchick, et al., supra”) are hereby incorporated by reference in their entirety.

In order to determine whether the mutant polypeptide is substantially identical with any vertebrate hormone of the GH-PRL_PL superfamily, the mutant polypeptide sequence can be aligned with the sequence of a first reference vertebrate hormone of that superfamily. One method of alignment is by BlastP, using the default setting for scoring matrix and gap penalties. In one embodiment, the first reference vertebrate hormone is the one for which such an alignment results in the lowest E value, that is, the lowest probability that an alignment with an alignment score as good or better would occur through chance alone. Alternatively, it is the one for which such alignment results in the highest percentage identity.

In general, the mutant polypeptide agonist is considered substantially identical to the reference vertebrate hormone if all of the differences can be justified as being (1) conservative substitutions of amino acids known to be preferentially exchanged in families of homologous proteins, (2) non-conservative substitutions of amino acid positions known or determinable (e.g., by virtue of alanine scanning mutagenesis) to be unlikely to result in the loss of the relevant biological activity, or (3) variations (substitutions, insertions, deletions) observed within the GH-PRL-PL superfamily (or, more particularly, within the relevant family). The mutant polypeptide antagonist will additionally differ from the reference vertebrate hormone by virtue of one or more receptor antagonizing mutations.

With regard to applying point (3) above to insertions and deletions, it is necessary to align the mutant polypeptide with at least two different reference hormones. This is done by pairwise alignment of each reference hormone to the mutant polypeptide.

When two sequences are aligned to each other, the alignment algorithm(s) may introduce gaps into one or both sequences. If there is a length one gap in sequence A corresponding to position X in sequence B, then we can say, equivalently, that (1) sequence A differs from sequence B by virtue of the deletion of the amino acid at position X in sequence B, or (2) sequence B differs from sequence A by virtue of the insertion of the amino acid at position X of sequence B, between the amino acids of sequence A which were aligned with positions X−1 and X+1 of sequence B.

If alignment of the mutant sequence to the first reference hormone creates a gap in the mutant sequence, then the mutant sequence can be characterized as differing from the first reference hormone by deletion of the amino acid at that position in the first reference hormone, and such deletion is justified under clause (3) if another reference hormone differs from the first reference hormone in the same way.

Likewise, if the alignment of the mutant sequence to the first reference hormone creates a gap in the reference sequence, then the mutant sequence can be characterized as differing from the first reference hormone by insertion of the amino acid aligned with that gap, and such insertion is justified under clause (3) if another reference hormone differs from the first reference hormone in the same way.

The preferred vertebrate GH-derived GH receptor agonists of the present invention are fusion proteins which comprise a polypeptide sequence P for which the differences, if any, between said amino acid sequence and the amino acid sequence of a first reference vertebrate growth hormone, are independently selected from the group consisting of

(a) a substitution of a conservative replacement amino acid for the corresponding first reference vertebrate growth hormone residue;
(b) a substitution of a non-conservative replacement amino acid for the corresponding first reference vertebrate growth hormone residue where
(i) another reference vertebrate growth hormone exists for which the corresponding amino acid is a non-conservative substitution for the corresponding first reference vertebrate growth hormone residue, and/or
(ii) the binding affinity of a single substitution mutant of the first reference vertebrate growth hormone, wherein said corresponding residue, which is not alanine, is replaced by alanine, is at least 10% of the binding affinity of the first vertebrate growth hormone for the vertebrate growth hormone receptor to which the first vertebrate growth hormone natively binds;
(c) a deletion of one or more residues found in said first reference vertebrate growth hormone but deleted in another reference vertebrate growth hormone;
(d) insertion of one or more residues into said first reference vertebrate growth hormone between adjacent amino acid positions of said first reference vertebrate growth hormone, where another reference vertebrate growth hormone exists which differs from said first reference growth hormone by virtue of an insertion at the same location of said first reference vertebrate growth hormone; and
(e) truncation of the first 1-8, 1-6, 1-4, or 1-3 residues and/or the last 1-8, 1-6, 1-4, or 1-3 residues found in said first reference vertebrate growth hormone (“truncation” is intended to refer to a deletion of residues at the N- or C-terminal of the peptide);
where the polypeptide sequence has at least 10% of the binding affinity of said first reference vertebrate growth hormone for a vertebrate growth hormone receptor, preferably one to which said first reference vertebrate growth hormone natively binds, and
where said fusion protein binds to and thereby activates a vertebrate growth hormone receptor.
We characterize the fusion protein as “GH-derived” because the polypeptide sequence P qualifies as a vertebrate GH or as a vertebrate GH mutant as defined above.

A growth hormone natively binds a growth hormone receptor found in the same species, i.e., human growth hormone natively binds a human growth hormone receptor, bovine growth hormone, a bovine GH receptor, and so forth.

For binding to the human growth hormone receptor, binding affinity is determined by the method described in Cunningham and Wells, “High-Resolution Mapping of hGH-Receptor Interactions by Alanine Scanning Mutagenesis”, Science 284: 1081 (1989), and thus uses the hGHRbp as the target. For binding to the human prolactin receptor, binding is determined by the method described in WO92/03478, and thus uses the hPRLbp as the target. For binding to nonhuman vertebrate hormone receptors, binding affinity is determined by use, in order of preference, of the extracellular binding domain of the receptor, the purified whole receptor, and an unpurified source of the receptor (e.g., a membrane preparation).

The receptor binding fusion protein preferably has growth promoting activity in a vertebrate. Growth promoting (or inhibitory) activity may be determined by the assays set forth in Kopchick, et al., which involve transgenic expression of the GH agonist or antagonist in mice. Or it may be determined by examining the effect of pharmaceutical administration of the GH agonist or antagonist to humans or nonhuman vertebrates.

Preferably, one or more of the following further conditions apply:

(1) the polypeptide sequence P is at least 50%, more preferably at least 55%, at least 60%, at least 65%, at least 70%, at least 75%, at least 80%, at least 85%, at least 90% or most preferably at least 95% identical to said first reference vertebrate growth hormone,
(2) the conservative replacement amino acids are highly conservative replacement amino acids,
(3) any deletion under clause (c) is of a residue which is not located at a conserved residue position of the vertebrate growth hormone family, and, more preferably is not a conserved residue position of the mammalian growth hormone subfamily,
(4) the first reference vertebrate growth hormone is a mammalian growth hormone, more preferably, a human or bovine growth hormone,
(5) any insertion under clause (d) is of a length such that another reference vertebrate growth hormone exists which differs from said first reference growth hormone by virtue of an equal length insertion at the same location of said first reference vertebrate growth hormone
(6) the differences are limited are limited to substitutions pursuant to clauses (a) and/or (b),
(7) if the first reference vertebrate growth hormone is a nonhuman growth hormone, and the intended use is in binding or activating the human growth hormone receptor, the differences increase the overall identity to human growth hormone,
(8) one or more of the substitutions are selected from the group consisting of one or more of the mutations characterizing the hGH mutants B2024 and/or B2036 as described below,
(9) the polypeptide sequence P is at least 50%, more preferably at least 55%, at least 60%, at least 65%, at least 70% at least 75%, at least 80%, at least 85%, at least 90%, at least 95% or, if an agonist, most preferably 100% similar to said first reference vertebrate growth hormone, or
(10) the polypeptide sequence P, when aligned to the first reference vertebrate growth hormone by BlastP using the Blosum62 matrix and the gap penalties −11 for gap creation and −1 for each gap extension, results in an alignment for which the E value is less than e-10, more preferably less than e-20, e-30, e-40, e-50, e-60, e-70, e-80, e-90 or most preferably e-100.

For purposes of condition (1), percentage identity is calculated by the BlastP methodology, i.e., identities as a percentage of the aligned overlap region including internal gaps. For purposes of condition (2), highly conservative amino acid replacements are as follows: Asp/Glu, Arg/His/Lys, Met/Leu/Ile/Val, and Phe/Tyr/Trp. For purposes of condition (3), the conserved residue positions are those which, when all vertebrate growth hormones whose sequences are in a publicly available sequence database as of the time of filing are aligned as taught herein, are occupied only by amino acids belonging to the same conservative substitution exchange group (I, II, III, IV or V) as defined above. The unconserved residue positions are those which are occupied by amino acids belonging to different exchange groups, and/or which are unoccupied (i.e., deleted) in one or more of the vertebrate growth hormones. The fully conserved residue positions of the vertebrate growth hormone family are those residue positions are occupied by the same amino acid in all of said vertebrate growth hormones. Clause (c) does not permit deletion of a residue at one of the fully conserved residue positions. One may analogously define fully conserved, conserved, and unconserved residue positions of the mammalian growth hormone family.

For purposes of condition (4), hGH is preferably the form of hGH which corresponds to the mature portion (AAs 27-217) of the sequence set forth in Swiss-Prot SOMA_HUMAN, P01241, isoform 1 (22 kDa), and bovine growth hormone is preferably the form of bovine growth hormone which corresponds to the mature portion (AA 28-217) of the sequence set forth in Swiss-Prot SOMA_BOVIN, P01246, per Miller W. L., Martial J. A., Baxter J. D.; “Molecular cloning of DNA complementary to bovine growth hormone mRNA.”; J. Biol. Chem. 255:7521-7524 (1980). These references are incorporated by reference in their entirety. For purpose of condition (10), percentage similarity is calculated by the BlastP methodology, i.e., positives (aligned pairs with a positive score in the Blosum62 matrix) as a percentage of the aligned overlap region including internal gaps.

Vertebrate GH-derived GH receptor antagonists of the present invention may be similarly defined, except that the polypeptide sequence must additionally differ from the sequence of the reference vertebrate growth hormone, e.g., at the position corresponding to Gly 119 in bovine growth hormone or Gly 120 in human growth hormone, in such manner as to impart GH receptor antagonist (binds but does not activate) activity to the polypeptide sequence and thereby to the fusion protein. Note that bGH Glyl 19/hGH Gly 120 is presently believed to be a fully conserved residue position in the vertebrate GH family. It has been reported that an independent mutation, R77c, can result in growth inhibition. See Takahashi Y, Kaji H, Okimura Y, Goji K, Abe H, Chihara K., “Brief report: short stature caused by a mutant growth hormone.”, N Engl J Med. 1996 Feb. 15; 334(7):432-6.

Preferably, the GH receptor antagonist has growth inhibitory activity. The compound is considered to be growth-inhibitory if the growth of test animals of at least one vertebrate species which are treated with the compound (or which have been genetically engineered to express it themselves) is significantly (at a 0.95 confidence level) slower than the growth of control animals (the term “significant” being used in its statistical sense). In some embodiments, it is growth-inhibitory in a plurality of species, or at least in humans and/or bovines.

Also, the GH antagonists may comprise an alpha helix essentially corresponding to the third major alpha helix of the first reference vertebrate growth hormone, and at least 50% identical (more preferably at least 80% identical) therewith. However, the mutations need not be limited to the third major alpha helix.

The contemplated vertebrate GH antagonists include, in particular, fusions in which the polypeptide P corresponds to the hGH mutants B2024 and B2036 as defined in U.S. Pat. No. 5,849,535. Note that B2024 and B2036 are both hGH mutants including, inter alia, a G10K substitution. In addition, we contemplate GH antagonists in which B2024 and B2036 are further mutated in accordance, mutatis mutandis, with the principles set forth above, i.e., in which B2024 or B2036 serves in place of a naturally occurring GH such as HGH as the reference vertebrate GH.

In a like manner, one may define vertebrate prolactin agonists and antagonists, and vertebrate placental lactogen agonists and antagonists, which agonize or antagonize a vertebrate prolactin receptor. One may also have mutants of a vertebrate growth hormone, which agonize or antagonize the prolactin receptor (with or without retention of activity against a growth hormone receptor), and mutants of a vertebrate prolactin or placental lactogen, which agonize or antagonize a vertebrate growth hormone receptor (with or without retention of activity against a prolactin receptor). In a like manner, one may define agonists and antagonists that are hybrids, or are mutants of hybrids, of two or more reference hormones of the vertebrate growth hormone—prolactin—placental lactogen hormone superfamily, and which retain at least 10% of at least one receptor binding activity of at least one of the reference hormones.

### Secondary Structure Prediction

Secondary structure prediction may be made by, e.g., Combet C., Blanchet C., Geourjon C. and Deléage G. “NPS@: Network Protein Sequence Analysis,” TIBS 2000 March Vol. 25, No 3 [291]:147-150, available online as the “HNN Secondary Structure Prediction Method” at Pole BioInformatique Lyonnais Network Protein Sequence Analysis, URL being http://npsa-pbil.ibcp.fr/cgi-bin/npsa_automat.pl?page=npsa_nn.html

### Use of Gene Ontology in the Definition of Classes of Proteins

The Gene Ontology Consortium has developed controlled vocabularies which describe gene products in terms of their associated biological processes, cellular components and molecular functions in a species-independent manner. For particulars, see http://www.geneontology.org/.

Formally speaking, the controlled vocabularies are specified in the form of three structured networks of controlled terms to describe gene product attributes. The three networks are molecular function, biological process, and cellular component. Each network is composed of terms of differing breadth. If term A is a subset of term B, then term A is the child of B and B is the parent of A.

In a given network, the terms are connected into a directed acyclic graph (DAG) structure, rather than a hierarchical structure. In a DAG, a child term can have more than one parent term. For example, the biological process term “hexose biosynthesis” has two parents, “hexose metabolism” and “monosaccharide biosynthesis”. This is because biosynthesis is a subtype of metabolism, and a hexose is a type of monosaccharide. If a child term describes the gene product, then all of its parents, must describe the gene product. And likewise all for the grandparents, great-grandparents, etc.

Molecular function describes the specific tasks performed by the gene product, i.e., its activities, such as catalytic or binding activities, at the molecular level. GO molecular function terms represent activities rather than the entities (molecules or complexes) that perform the actions, and do not specify where or when, or in what context, the action takes place. Molecular functions generally correspond to activities that can be performed by individual gene products, but some activities are performed by assembled complexes of gene products. Examples of broad functional terms are catalytic activity, transporter activity, or binding; examples of narrower functional terms are adenylate cyclase activity or Toll receptor binding.

Note that a single gene product might have several molecular functions, and many gene products can share a single molecular function. Hence, while gene products are often given names which set forth their molecular function, the use of a molecular function ontology term is meant to characterize the function of any gene product with that molecular function, not to refer to a particular gene product even if only one gene product is presently known to have that function.

Biological process describes the role of the gene product in achieving broad biological goals, such as mitosis or purine metabolism. A biological process is accomplished by one or more ordered assemblies of molecular functions. Examples of broad biological process terms are cell growth and maintenance or signal transduction. Examples of more specific terms are pyrimidine metabolism or alpha-glucoside transport. It can be difficult to distinguish between a biological process and a molecular function, but the general rule is that a process must have two or more distinct steps. Nonetheless, a biological process is not equivalent to a pathway, as the biological process ontologies do not attempt to capture any of the dynamics or dependencies that would be required to describe a pathway.

A cellular component is just that, a component of a cell but with the proviso that it is part of some larger object, which may be an anatomical structure (e.g. rough endoplasmic reticulum or nucleus) or a gene product group (e.g. ribosome, proteasome or a protein dimer).

GO does not contain the following:

Gene products: e.g. cytochrome c is not in the ontologies, but attributes of cytochrome c, such as electron transporter, are.

Processes, functions or components that are unique to mutants or diseases: e.g. oncogenesis is not a valid GO term because causing cancer is not the normal function of any gene.

Attributes of sequence such as intron/exon parameters: these are not attributes of gene products and will be described in a separate sequence ontology (see the OBO web page for more information).

Protein domains or structural features.

Protein-protein interactions.

The General Ontology data structures defines these ontology terms and their relationships. The data structures may be downloaded from the General Ontology Consortium website. A sample GO entry would be:

### id: GO:0045174

name: glutathione dehydrogenase (ascorbate) activity
xref_analog: EC:1.8.5.1 “ ”
def: “Catalysis of the reaction: 2 glutathione+dehydroascorbate=\glutathione disulfide+ascorbate.” [EC:1.8.5.1]
synonym: dehydroascorbate reductase [ ]
is_a: GO:0009055
is_a: GO:0015038
is_a: GO:0016672

Thus, it includes a GOid (the number has no significance other than that it is unique to that term), the name of the term, and, unless it is the root term of the network, identification of one or more immediate parents. These are identified by “is_a” if the parent need not comprise that child, and by “part_of” if the parent necessarily comprises that child. Cross-references and synonyms are optional.

To identify the gene ontology terms applicable to a particular gene product, one may search a collaborating database whose gene or gene product records have been annotated with one or more GOids. The annotation may include evidence codes to indicate the basis for assigning particular GOids to that gene or gene product.

For example, a search on in the NCBI Protein database

(accessible, e.g., at http://www.ncbi.nlm.nih.gov/entrez/query.fcgi?db=Gene)
generates an NCBI Sequence Viewer view which includes one or more function, process and component gene ontology entries for the query protein.

It will be appreciated that even if a particular mouse gene product or human gene product has not been annotated in a collaborating database, it is possible to determine its ontologies by considering the available evidence concerning its associated molecular functions, biological processes, and cellular components and classifying it according to the GO definitions in the same manner as was done by the collaborating database curators for the annotated genes.

The collaborating databases do not necessarily exhaustively annotate a gene. For example, if ontology A is child of B, and B is child of C, and C is child of D, and D is child of E, they may list the lower order ontologies A, B and C, but not the higher order ones D and E. It would, of course, be possible for a technician to examine all the terms in tables 3 and 4, determine which higher order ontologies have been omitted by comparing the terms with a complete directory of the gene ontology network, and add the missing higher order terms. We have not done this because, in general, the higher order ontologies, being less specific, are less likely to be of interest, at least taken by themselves.

For the purpose of the present invention, the possible predisposed proteins and Hyp-glycosylation-deficient parental proteins may be classified by gene ontology. Each gene ontology in the controlled vocabulary may be considered a separate embodiment. For example, one embodiment would relate to predisposed proteins with the function ontology of acyltransferase activity, and their expression and secretion in plants, another embodiment would be where the predisposed protein has the process ontology of cholesterol metabolism, a third where the predisposed protein has the component ontology of extracellular space. Likewise, the universe of predisposed proteins or of Hyp-glycosylation-deficient parental proteins, excluding proteins having one or more specified ontologies, may be considered disclosed embodiments.

As of Jul. 5, 2005, there were 9519 biological process, 1555 cellular component, and 7038 molecular function ontologies, for a total of 18112 ontologies. Thus, there are at least 18112 contemplated single ontology classes of predisposed proteins, and a like number of classes of Hyp-glycosylation-deficient proteins. We may similarly classify the Hyp-glycosylation-supplemented proteins; we assume that they have the same ontologies as the parental proteins until demonstrated otherwise. We may also define subclasses of predisposed and Hyp-glycosylation deficient proteins on the basis of combinations of two or more ontologies. There are three possible types of combinations to be considered: a) combinations of ontologies in which each ontology is from a different network (i.e., molecular function, biological process, biological component); b) combinations of ontologies in which each ontology is from the same network, but in which no ontology is a child or a parent of any other ontology in the same combination; and c) combinations of ontologies which include ontologies from more than one network, as well as more than one ontology from the same network, but where no ontology is a child or a parent of any other ontology in the same combination.

### Secretion Signal Peptides

For secretion in plants, a nucleic acid construct is designed which encodes a precursor protein consisting of an N-terminal signal peptide which is functional in the plant cell of interest, followed by the amino acid sequence of the mature protein of interest (which may but need not be a mutant protein). The precursor protein is expressed and, as it is secreted through the membrane, the signal peptide is cleaved off.

In the discussion which follows, the abbreviation TSP means total soluble protein. Preferably, the secretion signal peptide is one which, in the plant cell in question, can achieve secretion of a non-Hyp-glycosylated protein at a level of at least 0.01% TSP., more preferably at least 0.1% TSP, still more preferably at least 0.5% TSP, most preferably at least 1% TSP.

In one series of embodiments, the signal peptide is one native to a plant protein, including but not limited to one of the following:

### 1. Tobacco Extensin Signal Peptide

Previously used in our lab (Shpak et al., PNAS 96:14736-14741, 1999, Xu et al., Biotechnol. Bioeng. 90:578-588, 2005) to secrete EGFP, interferon alpha2b, human serum albumin, and human growth hormone.

### 2. Arabidopsis Basic Chitinase Signal Peptide

Previously used to secrete GFP (Tobacco cell suspension culture, CaMV 35S promoter, 50% secreted, 12 mg/L; Su et al., High-level secretion of functional green fluorescent protein from transgenic tobacco cell cultures. Biotechnol. Bioeng. 85, 610-619, 2004).

### 3. Tobacco PR (Pathogen-Related)-S Signal Peptide

Previously used to secrete human serum albumin (tobacco leaves chloroplasts, 11% TSP, Plant Biotechnol. J. 1, 71-79, 2003; Potato and tobacco plant, CaMV 35S promoter, 0.02% TSP, Sijmons et al., Bio/Technology, 8:217-221, 1990)

### 4. Ramy3D Signal Peptide

Previously used to secrete Human granulocyte-macrophage colony-stimulating factor (hGM-CSF) (Rice cell suspension culture, Ramy3D promoter, secreted 125 mg/L; Shin et al., Biotechnol. Bioeng. 82 (7): 778-783, 2003)

### 5. Chloroplastic Transit Signal Peptide

Previously used to secrete human hemoglobin (Tobacco plant, CaMV35S promoter, 0.05% TSP in seed, Dieryck et al., Nature 386 (6620): 29-30, 1997)

### 6. Tobacco AP24 Osmotin Signal Peptide

Previously used to secrete human epidermal growth factor (Tobacco plant, CaMV35S promoter or CaMV 35S long promoter, 0.015% TSP, Wirth et al., MOLECULAR BREEDING 13 (1): 23-35, 2004)

### 7. Alpha-Coixin Signal Peptide

Previously used to secrete Human growth hormone (Tobacco seed, sorghum gamma-kafirin gene promoter, 0.16% TSP, Leite et al., MOLECULAR BREEDING 6 (1): 47-53, 2000; Tobacco chloroplasts, 7% TSP, Staub et al., Nature Biotechnol. 18 (3): 333-338, 2000)

### 8. Lam B Signal Peptide

Previously used to secrete Human insulin-like growth factor (Tobacco plant, Maize ubiquitin promoter, 43 ng/mg TSP, Panahi et al., Molecular Breeding, 12:21-31, 2003)

### 9. Barley Alpha-Amylase Signal Peptide

Previously used to secrete Aprotinin (Maize seeds, maize ubiquitin promoter, 0.07% TSP, Zhong et al., MOLECULAR BREEDING 5 (4): 345-356, 1999)
Alternatively, in a second series of embodiments, the signal peptide associated with a secreted plant virus protein is employed. For example, it may be the TMV omega coat protein signal peptide.
Alternatively, in a third series of embodiments, the non-plant protein's native signal peptide is used to achieve secretion in plants. (If the protein is a modified protein, then we are referring to the signal peptide of the most closely related naturally occurring protein.) Many non-plant eukaryotic signals are functional in plants; examples are given below:
1. Human milk β-casein (Solanum tuberosum (Potato) leaves, Auxin-inducible mannopine synthase promoter, native signal peptide, 0.01% TSP, Chong et al., Transgenic Res., 6, 289-296, 1997)
2. Human milk CD14 protein (Tobacco cell culture, CaMV35S promoter, native signal sequence or tomato extensin signal peptide, 5 ug/L medium, Girard et al., Plant Cell, Tissue and Organ Culture 78: 253-260, 2004)
3. Human interferon beta (Tobacco plant, CaMV35S promoter, native signal peptide, 0.01% fresh weight, J. Interferon Res. 12 (6): 449-453, 1992)
4. Human Interleukin-2 (Tobacco cell culture, CaMV35S promoter, native signal peptide, secreted, 0.1 ug/L, Magnuson et al., Protein Expr. Purifi. 13 (1): 45-52, 1998)
5. Human muscarinic cholinergic receptors (Tobacco plant and BY-2 cell culture, CaMV35S promoter, native signal peptide, 240 fmol/mg membrane protein. Mu et al., Plant Mol. Bio. 34 (2): 357-362, 1997)
6. Phytase (Tobacco plant, CaMV35S promoter, native signal peptide, 14.4% TSP, Verwoerd et Al., Plant Physiology 109 (4): 1199-1205, 1995)
7. Xylanase (Tobacco plant, CaMV35S promoter, native signal peptide, 4.1% TSP leaves, Herbers et al., Bio/Technolo. 13 (1): 63-66, 1995)
8. Heat-labile enterotoxin B subunit (Potato plant, CaMV35S promoter, native signal peptide, 0.01% TSP, Mason et al., vaccine 16(3):1336-1343, 1996)
9. Norwalk virus capsid protein (Tobacco leaves and potato tubers, CaMV35S promoter or patatin promoter, native signal peptide, 0.23% TSP, Mason et al., PNAS, 93 (11): 5335-5340, 1996)
10. Cholera toxin B subunit (Tomato plant, CaMV35S promoter, native signal peptide, 0.02%-0.04% TSP, Jani et al., Transgenic Res. 11 (5): 447-454, 2002; Tobacco plant, ubiquitin promoter, native signal peptide, 1.8% TSP, Kang et al., Molecular Biotechnology 32 (2): 93-100, 2006)
If the foreign protein is a chimeric protein, then the native signal could be the one native to either of the parental proteins, but normally the one native to the N-terminal domain would be preferred.
In a fourth series of embodiments, the signal peptide is a signal, functional in plants, which is neither the native signal of the foreign protein, nor one native to plants. or plant viruses.
Murine immunoglobulin signal peptide was previously used to secrete HIV-1 p24 antigen fused to human IgA (Tobacco plant, CaMV35S promoter, 1.4% TSP, Obregon, et al., Plant Biotechnol. J. 4(2): 195-207 (2006). The Obregon murine immunoglobulin signal peptide was also able to direct secretion of unfused HIV-1 p24 antigen, but secretion was at a level of 0.1% TSP.

### Non-Hyp Glycosylation

While we are primarily concerned with Hyp-glycosylation, other forms of glycosylation may contribute to secretion, solubility, stability, etc., and hence it is helpful to identify sites for such other forms. In some embodiments, the carbohydrate component of the glycoprotein, including both Hyp-glycosylation and optionally other glycosylation, accounts for at least 10% of the molecular weight of the protein.

### O-Glycosylation at Other Amino Acids

In general (that is, without limitation to plant proteins), O-glycosylation occurs at Ser, Thr, Tyr, and Hyl, as well as at Hyp. GlcNAc, GalNAc, Gal, Man, Fuc, Pse, DiAcTridH, Glc, FucNac, Xyl and Gal are reported to O-link to Ser, and GlcNAc, GalNAc, Gal, Man, Fuc, Pse, DiAcTridH, Glc and Gal to Thr. GlcNAc, Gal and Ara are found on Hyp, Gal on Hyl, and Gal and Glc on Tyr. Spiro Table III provides consensus sequences for some of these glycosylation sites.

The proteins of the present invention may optionally include one or more O-glycosylated amino acids other than Hyp.

### N-Glycosylation

In proteins generally, N-glycosylation occurs at Asn or Arg. The principal sugar-peptide bonds identified are of GlcNAc, GalNAc, Glc and Rha to Asn, and of Glc to Arg. The consensus sequence for attachment of GlcNAc to Asn is Asn-Xaa-Ser/Thr (i.e., an “NAS” or “NAT”, where Xaa is any amino acid except Pro.

The proteins of the present invention may optionally include one or more N-glycosylated amino acids. These N-glycosylation sites may be native to the protein and/or the result of genetic engineering. Genetic engineering of sites may involve the introduction of Asn or Arg by substitution and/or insertion, and/or the modification of nearby amino acids to increase the probability of N-glycosylation of Asn or Arg.

For example, an NAS or NAT N-glycosylation motif may be provided at the N-terminal or C-terminal of the engineered protein. This could be provided by any means, including pure addition, partial addition (e.g., the native amino-terminal residue was already S or T or the native carboxy-terminal residue were already N), a combination of addition and substitution (e.g., changing the ammo terminal residue to S and then inserting NA in front of it), or pure substitution (e.g., replacing the first three residues with NAS or NAT).

Many plant extracellular proteins are N-glycosylated by the covalent linkage of glycans to asparagine (Asn) residues at Asn-X-Ser/Thr concensus sequence (Driouich et al., 1989). The physiological function of N-glycosylation is thought to involve adjusting protein structure for secretion (Okushima et al., 1999). From results obtained in previous studies on protein secretion in plant cells, it appears that N-glycosylation is a prerequisite for transport of proteins from ER to Golgi apparatus, and finally to extracellular space. Enhanced secretion of heterologous proteins was also found in yeast by introduction of an N-glycosylation site (Sagt et al., 2000). As a consequence, a specific N-glycan, or peripheral glycan epitopes, might be involved in protein targeting to the extracellular compartment.

### See

Driouich A, Gonnet P, Makkie M, Laine A-C and Faye L. (1989) The role of high-mannose and complex asparagines-linked glycans in the secretion and stability of glycaproteins. Planta 180:96-104.
Olden, K., Parent, J. B., White, S. J. (1982) Carbohydrate moieties of glycoproteins: A re-evaluation of their function. Biochim. Biophys. Acta 650:209-232.
Okushima Y, Koizumi N, Sano H. 1999. Glycosylation and its adquent processing is critical for protein secretion in tobacco BY2 cells. J Plant Physiol. 154: 623-627.
Fiedler K and Simons K. (1995) The role of N-glycans in the secretory pathway. Cell 81:309-312.
Sagt C M J, Kleizen B, Verwaal R, DeJong M D W, Muller W H, Smits A, Visser C, Boonstra J, Verkleij A J and Verrips C T. (2000) Introduction of an N-glycosylation site increases secretion of heterologous protein in yeast. Appl. Environ. Microbiol. 66:4949-4944.

### Deglycosylation

In some cases, glycosylation is desirable to improve secretion or to facilitate purification, but is not required in the protein for clinical use. After expression and secretion, the glycoproteins may be deglycosylated, e.g., to improve their biological activity. Deglycosylating agents may be enzymatic (e.g., peptide N-glycosidase F, “PNGase F”, or endo-beta-N-acetylglucosaminidase H, “endo H”) or chemical (e.g., trifluoromethanesulfonic acid; periodate; anhydrous hydrogen fluoride).

### Expression in Plants

The recombinant genes are expressed in plant cells, such as cell suspension cultured cells, including but not limited to, BY2 tobacco cells. Expression can also be achieved in a range of intact plant hosts, and other organisms including but not limited to, invertebrates, plants, sponges, bacteria, fungi, algae, archebacteria.

In some embodiments, the expression construct/plasmid/recombinant DNA comprises a promoter. It is not intended that the present invention be limited to a particular promoter. Any promoter sequence which is capable of directing expression of an operably linked nucleic acid sequence encoding at least a portion of nucleic acids of the present invention, is contemplated to be within the scope of the invention. Promoters include, but are not limited to, promoter sequences of bacterial, viral and plant origins. Promoters of bacterial origin include, but are not limited to, octopine synthase promoter, nopaline synthase promoter, and other promoters derived from native Ti plasmids. Viral promoters include, but are not limited to, 35S and 19S RNA promoters of cauliflower mosaic virus (CaMV), and T-DNA promoters from Agrobacterium. Plant promoters include, but are not limited to, ribulose-1,3-bisphosphate carboxylase small subunit promoter, maize ubiquitin promoters, phaseolin promoter, E8 promoter, and Tob7 promoter.

The invention is not limited to the number of promoters used to control expression of a nucleic acid sequence of interest. Any number of promoters may be used so long as expression of the nucleic acid sequence of interest is controlled in a desired manner. Furthermore, the selection of a promoter may be governed by the desirability that expression be over the whole plant, or localized to selected tissues of the plant, e.g., root, leaves, fruit, etc. For example, promoters active in flowers are known (Benfy et al. (1990) Plant Cell 2:849-856).

Transformation of plant cells may be accomplished by a variety of methods, examples of which are known in the art, and include for example, particle mediated gene transfer (see, e.g., U.S. Pat. No. 5,584,807 hereby incorporated by reference); infection with an Agrobacterium strain containing the foreign DNA-for random integration (U.S. Pat. No. 4,940,838 hereby incorporated by reference) or targeted integration (U.S. Pat. No. 5,501,967 hereby incorporated by reference) of the foreign DNA into the plant cell genome; electroinjection (Nan et al. (1995) In “Biotechnology in Agriculture and Forestry,” Ed. Y. P. S. Bajaj, Springer-Verlag Berlin Heidelberg, Vol 34:145-155; Griesbach (1992) HortScience 27:620); fusion with liposomes, lysosomes, cells, minicells, or other fusible lipid-surfaced bodies (Fraley et al. (1982) Proc. Natl. Acad. Sci. USA 79:1859-1863; polyethylene glycol (Krens et al. (1982) Nature 296:72-74); chemicals that increase free DNA uptake; transformation using virus, and the like.

The terms “infecting” and “infection” with a bacterium refer to co-incubation of a target biological sample, (e.g., cell, tissue, etc.) with the bacterium under conditions such that nucleic acid sequences contained within the bacterium are introduced into one or more cells of the target biological sample.

The term “Agrobacterium” refers to a soil-borne, Gram-negative, rod-shaped phytopathogenic bacterium, which causes crown gall. The term “Agrobacterium” includes, but is not limited to, the strains Agrobacterium tumefaciens, (which typically causes crown gall in infected plants), and Agrobacterium rhizogenes (which causes hairy root disease in infected host plants). Infection of a plant cell with Agrobacterium generally results in the production of opines (e.g., nopaline, agropine, octopine, etc.) by the infected cell. Thus, Agrobacterium strains which cause production of nopaline (e.g., strain LBA4301, C58, A208) are referred to as “nopaline-type” Agrobacteria; Agrobacterium strains which cause production of octopine (e.g., strain LBA4404, Ach5, B6) are referred to as “octopine-type” Agrobacteria; and Agrobacterium strains which cause production of agropine (e.g., strain EHA105, EHA101, A281) are referred to as “agropine-type” Agrobacteria.

The terms “bombarding,” “bombardment,” and “biolistic bombardment” refer to the process of accelerating particles towards a target biological sample (e.g., cell, tissue, etc.) to effect wounding of the cell membrane of a cell in the target biological sample and/or entry of the particles into the target biological sample. Methods for biolistic bombardment are known in the art (e.g., U.S. Pat. No. 5,584,807, the contents of which are herein incorporated by reference), and are commercially available (e.g., the helium gas-driven microprojectile accelerator (PDS-1000/He) (BioRad).

The term “microwounding” when made in reference to plant tissue refers to the introduction of microscopic wounds in that tissue. Microwounding may be achieved by, for example, particle, or biolistic bombardment.

Plant cells can also be transformed according to the present invention through chloroplast genetic engineering, a process that is described in the art. Methods for chloroplast genetic engineering can be performed as described, for example, in U.S. Pat. No. 6,680,426, and in published U.S. Application Nos. 2003/0009783, 2003/0204864, 2003/0041353, 2002/0174453, 2002/0162135, the entire contents of each of which is incorporated herein by reference.

It is not intended that the present invention be limited by the host cells used for expression of the synthetic genes of the present invention, provided that they are plant cells capable of hydroxylating proline and of glycosylating (especially arabinosylating or arabinogalactosylating) hydroxyproline.

Plants that can be used as host cells include vascular and non-vascular plants. Non-vascular plants include, but are not limited to, Bryophytes, which further include but are not limited to, mosses (Bryophyta), liverworts (Hepaticophyta), and hornworts (Anthocerotophyta). Other cells contemplated to be within the scope of this invention are green algae types, such as Chlamydomonas and Volvox.

Vascular plants include, but are not limited to, lower (e.g., spore-dispersing) vascular plants, such as, Lycophyta (club mosses), including Lycopodiae, Selaginellae, and Isoetae, horsetails or equisetum (Sphenophyta), whisk ferns (Psilotophyta), and ferns (Pterophyta).

Vascular plants further include, but are not limited to, i) fossil seed ferns (Pteridophyta), ii) gymnosperms (seed not protected by a fruit), such as Cycadophyta (Cycads), Coniferophyta (Conifers, such as pine, spruce, fir, hemlock, yew), Ginkgophyta (e.g., Ginkgo), Gnetophyta (e.g., Gnetum, Ephedra, and Welwitschia), and iii) angiosperms (flowering plants—seed protected by a fruit), which includes Anthophyta, further comprising dicotyledons (dicots) and monocotyledons (monocots). Specific plant host cells that can be used in accordance with the invention include, but are not limited to, legumes (e.g., soybeans) and solanaceous plants (e.g., tobacco, tomato, etc.).

The monocots of interest include Poaceae/Graminaceae (e.g., rice, maize, wheat, barley, rye, oats, millet, sugarcane, sorghum, bamboo), Araceae (e.g., Anthurium, Zantedeschia, taro, elephant ear, Dieffenbachia, Monstera, Philodendron), including those of the old classification Lemnaceae (e.g., duckweed (Lemna)), Orchidaceae (e.g., various orchids), and Cyperaceae (e.g., various sedges).

The dicots of interest may be eudicots or paleodicots, and include Solanaceae (e.g., potato, tobacco, tomato, pepper), Fabaceae (e.g., beans, peas, peanuts, soybeans, lentils, lupins, clover, alfalfa, cassia), Cucurbitaceae (e.g., squash, pumpkin, melon, cucumber), Rosaceae (e.g., apple, pear, cherry, apricot, plum, rose, raspberry, strawberry, hawthorn, quince, peach, almond, rowan, hawthorn), Brassicaceae (e.g., cabbage, broccoli, cauliflower, brussels sprouts, collards, kale, Chinese kale, rutabaga, seakale, turnip, radish, kohlrabi, rapeseed, mustard, horseradish, wasabi, watercress, Arabidopsis “rockcress”), Asteraceae (e.g., lettuce, chicory, globe artichoke, sunflower, Jerusalem artichoke), Rubiaceae (e.g., madder, bedstraw, cffee, cinchona, partridgeberry, gambier, ixora, noni), Euphorbiaceae (e.g. spurge, manioc, castor bean, para rubber, poinsettia), and Malvaceae (e.g., mallows, cotton plants, okra, hibiscus, hollyhocks).

The present invention is not limited by the nature of the plant cells. All sources of plant tissue are contemplated. In one embodiment, the plant tissue which is selected as a target for transformation with vectors which are capable of expressing the invention's sequences are capable of regenerating a plant. The term “regeneration” as used herein, means growing a whole plant from a plant cell, a group of plant cells, a plant part or a plant piece (e.g., from seed, a protoplast, callus, protocorm-like body, or tissue part). Such tissues include but are not limited to seeds. Seeds of flowering plants consist of an embryo, a seed coat, and stored food. When fully formed, the embryo generally consists of a hypocotyl-root axis bearing either one or two cotyledons and an apical meristem at the shoot apex and at the root apex. The cotyledons of most dicotyledons are fleshy and contain the stored food of the seed. In other dicotyledons and most monocotyledons, food is stored in the endosperm and the cotyledons function to absorb the simpler compounds resulting from the digestion of the food.

Species from the following examples of genera of plants may be regenerated from transformed protoplasts: Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Hyoscyamus, Lycopersicon, Nicotiana, Solanum, Petunia, Digitalis, Majorana, Ciohorium, Helianthus, Lactuca, Bromus, Asparagus, Antirrhinum, Hererocallis, Nemesia, Pelargonium, Panicum, Pennisetum, Ranunculus, Senecio, Salpiglossis, Cucunis, Browaalia, Glycine, Lolium, Zea, Triticum, Sorghum, and Datura.

For regeneration of transgenic plants from transgenic protoplasts, a suspension of transformed protoplasts or a petri plate containing transformed explants is first provided. Callus tissue is formed and shoots may be induced from callus and subsequently rooted. Alternatively, somatic embryo formation can be induced in the callus tissue. These somatic embryos germinate as natural embryos to form plants. The culture media will generally contain various amino acids and plant hormones, such as auxin and cytokinins. It is also advantageous to add glutamic acid and proline to the medium, especially for such species as corn and alfalfa. Efficient regeneration will depend on the medium, on the genotype, and on the history of the culture. These three variables may be empirically controlled to result in reproducible regeneration.

Plants may also be regenerated from cultured cells or tissues. Dicotyledonous plants which have been shown capable of regeneration from transformed individual cells to obtain transgenic whole plants include, for example, apple (Malus pumila), blackberry (Rubus), Blackberry/raspberry hybrid (Rubus), red raspberry (Rubus), carrot (Daucus carota), cauliflower (Brassica oleracea), celery (Apium graveolens), cucumber. (Cucumis sativus), eggplant (Solanum melongena), lettuce (Lactuca sativa), potato (Solanum tuberosum), rape (Brassica napus), wild soybean (Glycine canescens), strawberry (Fragaria×ananassa), tomato (Lycopersicon esculentum), walnut (Juglans regia), melon (Cucumis melo), grape (Vitis vinifera), and mango (Mangifera indica). Monocotyledonous plants which have been shown capable of regeneration from transformed individual cells to obtain transgenic whole plants include, for example, rice (Oryza sativa), rye (Secale cereale), and maize.

In addition, regeneration of whole plants from cells (not necessarily transformed) has also been observed in: apricot (Prunus armeniaca), asparagus (Asparagus officinalis), banana (hybrid Musa), bean (Phaseolus vulgaris), cherry (hybrid Prunus), grape (Vitis vinifera), mango (Mangifera indica), melon (Cucumis melo), ochra (Abelmoschus esculentus), onion (hybrid Allium), orange (Citrus sinensis), papaya (Carrica papaya), peach (Prunus persica), plum (Prunus domestica), pear (Pyrus communis), pineapple (Ananas comosus), watermelon (Citrullus vulgaris), and wheat (Triticum aestivum).

The regenerated plants are transferred to standard soil conditions and cultivated in a conventional manner. After the expression vector is stably incorporated into regenerated transgenic plants, it can be transferred to other plants by vegetative propagation or by sexual crossing. For example, in vegetatively propagated crops, the mature transgenic plants are propagated by the taking of cuttings or by tissue culture techniques to produce multiple identical plants. In seed propagated crops, the mature transgenic plants are self crossed to produce a homozygous inbred plant which is capable of passing the transgene to its progeny by Mendelian inheritance. The inbred plant produces seed containing the nucleic acid sequence of interest. These seeds can be grown to produce plants that would produce the desired polypeptides. The inbred plants can also be used to develop new hybrids by crossing the inbred plant with another inbred plant to produce a hybrid.

It is not intended that the present invention be limited to only certain types of plants. Both monocotyledons and dicotyledons are contemplated. Monocotyledons include grasses, lilies, irises, orchids, cattails, palms, Zea mays (such as corn), rice barley, wheat and all grasses. Dicotyledons include almost all the familiar trees and shrubs (other than confers) and many of the herbs (non-woody plants).

Tomato cultures are one example of a recipient for repetitive HRGP modules to be hydroxylated and glycosylated. The cultures produce cell surface HRGPs in high yields easily eluted from the cell surface of intact cells and they possess the required posttranslational enzymes unique to plants—HRGP prolyl hydroxylases, hydroxyproline O-glycosyltransferases and other specific glycosyltransferases for building complex polysaccharide side chains. Other recipients for the invention's sequences include, but are not limited to, tobacco cultured cells and plants, e.g., tobacco BY 2 (bright yellow 2).

### EXPERIMENTAL EXAMPLES

Experimental examples showing the expression and secretion, in tobacco cells, of non-plant proteins modified to include addition or insertion glycomodules are set forth in the examples of the prior related applications, incorporated by reference in their entirety.

### Hypothetical Example

### Protocol for Agrobacterium Mediated Transformation of Duckweed (Lemna minor) with the hGH-(SP)10 Gene (Yamamoto, et al., 2001) and Isolation of hGH-(SO)10

### Callus Induction and Nodule Production

1. Surface sterilize Lemna minor with 5% Clorox, then maintain the plant in liquid Schenk and Hildebrandt (SH) (Schenk and Hildebrandt, 1972) medium containing 10 g/L sucrose (pH 5.6) at 23° C. under continuous white florescent light (about 30-40 ìmol/m2 per second).

2. Incubate 5-6 fronds of Lemna minor from approximately 2-week-old cultures on a Petri dish containing 25 ml callus induction medium: MS basal salts, 30 g/L sucrose, 5 ?M 2,4-dichlorophenoxyacetic acid (2,4-D), 0.5 ?M thidiazuron and 2 g/L Phytagel (Sigma) (pH 5.6).

3. Pick up small white callus after 6 weeks and subculture on nodule production (NP) medium: MS basal salts, 30 g/L sucrose, 1 ?M 2,4-D, 2 ?M 6-benzoyladenine, and 2 g/L phytagel (pH 5.6). Nodules will be produced from callus after 2 weeks and were used for transformation or transferred to fresh NP medium every 2 weeks for future use. (Nodules are partially organized light green cell masses).

### Transformation of Nodules

1. Grow the Agrobacterium tumefaciens (LBA4404) harboring pBI121-hGH-(SP)10 vector at 28° C. overnight on a LB medium containing 50 mg/L kanamycin, 40 mg/L streptomycin and 100 ?M acetosyringone until OD595=1.0.

2. Collect the bacteria by centrifugation at 3000 g for 5 min, then re-suspend the bacteria in the same volume of re-suspension medium: MS salts, 0.6 M mannitol and 100 ?M acetosyringone (pH 5.6), and incubate for at least 1 hr at room temperature.

3. Submerge healthy, rapidly growing nodules that are approximately 3 mm in diameter in the bacterial suspension for 3-5 min.

4. Place the nodules on NP medium containing 100 ?M acetosyringone (10 nodules per Petri dish) and incubate for 2 days in the dark at 23° C.

5. Transfer the nodules to selective NP medium that contains 100 mg/L kanamycin and 400 mg/L timentin (SmithKline Beecham, PA), and incubate for 4 weeks in subdued light approximately 4 mmol/m2 per second. (Transfer the nodules weekly to fresh selective NP medium during this time).

6. Incubate the nodules under full light on selective NP medium for 2 weeks or until selected nodules are distinct. Then transfer the selected healthy nodules to fresh selective NP medium and incubate for another 2 weeks.

7. Induce regeneration of frond by incubating selected nodules on frond regeneration (FR) medium: half-strength SH with 5 g/L sucrose and 2 g/L phytagel (pH 5.6). Inclusion of 100 mg/L kanamycine in the FR medium is recommended.

8. Transfer the regenerated fronds into liquid SH medium.

### An Alternative Protocol for Nodule Transformation

1-4. Same as above

5. Transfer each nodule into a 125 ml flasks containing 40 ml SH medium with 10 g/L sucrose, 5 mg/L kanamycine and 400 mg/L timentin and incubate on a rotary shaker at 100 rpm at 23° C. Change the medium weekly.

6. Pick one regenerated frond from each flask to establish an independent transgenic line.

Isolation of hGH-(SO)10

1. Culture 15-20 regenerated fronds in vented containers containing 100 ml SH medium (without sucrose) at 23° C. under continuous white florescent light (about 30-40 ìmol/m2 per second).

2. Collect the medium after 2-3 weeks of culture by filtration on a coarse sintered funnel and add sodium chloride in the medium to a final concentration of 2 M.

3. Remove the insoluble materials of the medium by centrifugation at 25,000×G for 20 min at 4° C.

4. Load the supernatant onto a hydrophobic-interaction chromatography (HIC) column (Phenyl-Sepharose 6 Fast Flow, 16?700 mm, Amersham Pharmacia Biotech) equilibrated in 2 M sodium chloride at a flow rate of 1.5 ml/min.

5. Elute the proteins step-wise first with 25 mM Tris buffer (pH8.5)/2N NaCl, followed by Tris buffer/0.8 N NaCl, and then Tris buffer/0.2 N NaCl. Monitor the fractions at 220 nm with a UV detector.

6. Collect the Tris buffer/0.2 N NaCl fraction containing most of the hGH-(SO)10 protein and concentrate by ultrafiltration at 4° C. before performing hGH binding and activity assays.

7. Further purify hGH-(SO)10 by reversed phase chromatography on a Hamilton polymeric reversed phase-1 (PRP-1) analytical column (4.1?150 mm, Hamilton Co., Reno, Nev.) equilibrated with buffer A (0.1% trifluoroacetic acid). Elute the proteins with buffer B (0.1% trifluoroacetic acid, 80% acetonitrile, v/v) using a two step linear gradient of 0-30% B in 15 min, followed by 30%-70% B in 90 min at a flow rate of 0.5 ml/min. Measured the absorbance at 220 nm.

### REFERENCES FOR DUCK-WEED EXAMPLE

Schenk, R. U. and Hildebrandt, A. C. (1972) Medium and techniques for induction and growth of monocotyledonous and dicotyledonous plant cell cultures. Can J Bot, 50:199-204.
Yamamoto, Y. T. et al. (2001) Genetic transformation of duckweed Lemna Gibba and Lemna Minor. In Vitro Cell. Dev. Bio.-Plant 37:349-353.

### Miscellaneous

As used herein, “peptide,” “polypeptide,” and “protein,” can and will be used interchangeably. “Peptide/polypeptide/protein” will occasionally be used to refer to any of the three, but recitations of any of the three contemplate the other two. That is, there is no intended limit on the size of the amino acid polymer (peptide, polypeptide, or protein), that can be expressed using the present invention. Additionally, the recitation of “protein” is intended to encompass enzymes, hormone, receptors, channels, intracellular signaling molecules, and proteins with other functions. Multimeric proteins can also be made in accordance with the present invention.

### EXAMPLES

Using the default algorithm described above, we have predicted the sites of proline hydroxylation and hydroxyproline glycosylation for various non-plant proteins, if expressed in plants.

The signal peptide sequence is italicized. Please note that the prolines in the signal sequence should not be considered targets for hydroxylation and glycosylation. Note that there is sometimes uncertainty as to the exact bounds of the signal sequence. If in doubt, you can search on each of the putative mature sequences.

Predictions as to hydroxylation and glycosylation are indicated as follows: Arabinogalactosylated Hyp is #; Arabinosylated Hyp is @; Non-glycosylated Hyp is O; Non-hydroxylated Pro is P. Hydroxylation will not be 100%, nor will every Hyp residue be glycosylated.

The preliminary predictive methods set forth above are biased toward over-prediction, i.e., they are more likely to produce false positives than false negatives. Consequently, the skilled worker may wish to more closely evaluate each predicted Pro-Hydroxylation/Hyp-Glycosylation site, e.g., comparing it to known plant Hyp-glycomodules, considering the known or predicted secondary, supersecondary or tertiary structure, etc.

As an example of how such an evaluation might proceed, we present the preliminary predictions for a substantial number of proteins below, together with comments.

Several proteins with predicted Hyp-glycosylation sites (Pro-hydroxylation predicted by the quantitative method using the new matrix; Hyp-glycosylation predicted using the new standard method, i.e., tests A-O) have been classified below into Category I (probable Hyp-glycosylation when expressed in plants), Category II (Hyp-glycosylation possible, but less likely than for I), or Category III (Hyp-glycosylation unlikely despite the prediction), as a result of such a closer evaluation. (The Category III listing also includes several proteins for which the preliminary method predicted that Hyp-glycosylation sites would not exist.)

It must be emphasized that this three-way classification is a subjective one. It is merely an appraisal, based on consideration of many factors, of the likelihood that Hyp-glycosylation will in fact be observed if these proteins were expressed in plant cells. The factors considered include (or can include)
the number of predicted Hyp-glycosylation sites
the location of those predicted Hyp-glycosylation sites relative to the termini (which are likely to be more flexible) and relative to cysteines participating in known or predictable disulfide bonds
the richness of the vicinity (within 2-10 aa on either side, with perhaps more weight given to the nearer amino acids, especially those within 5 aa on either side) of those sites in proline (in the translated sequence) (proline will tend to result in an extended conformation and thus may facilitate the presentation of the predicted Pro-hydroxylation or Hyp-glycosylation site to enzymes)
the richness of the vicinity (ditto) of those sites in Ser, Ala, and Thr, and perhaps also in Val (For example, one might look for a 4-5 amino acid stretch that is at least 20%, more preferably at least 30%. Pro/Ser/Ala/Thr/Val, or better yet Pro/Ser/Ala/Thr)
the known or predicted secondary, supersecondary, or tertiary structure of the protein at the site and in the vicinity of the site.

Likewise, in identifying mutations likely to convert a category III parental protein into a modified protein with at least one actual Hyp-glycosylation site, both the considerations underlying the preliminary methods, and those mentioned in this section, were or could be considered. In addition, one may consider
which residues are conserved within the family of homologous proteins to which the parental protein belongs,
regions known to be involved in the biological activity of the parental protein
the properties of known mutants of the parental protein
the known or predicted secondary, supersecondary or tertiary structure of the parental protein.

No attempt has been made to be comprehensive in identifying suitable mutations.

I. Non-Plant Proteins with Predicted Pro Hydroxylation/Hyp Glycosylation Sites when Expressed in Plants.

### Adrenomedullin (NP001115.1)

(SEQ ID NO: 6)

MKLVSVALMY LGSLAFLGAD TARLDVASEF RKKWNKWALS
RGKRELRMSS SYPTGLADVK AGOAQTLIRP QDMKGASRSO
EDSS#DAARI RVKRYRQSMN NFQGLRSFGC RFGTCTVQKL
AHQIYQFTDK DKDNVAORSK ISOQGYGRRR RRSLPEAGPG
RTLVSSKPQA HGA#A@OSGS AOHFL_

### Atrial Natiuretic Factor (NM006172.1)

(SEQ ID NO: 7)

MSSFSTTTVS FLLLLAFQLL GQTRANPMYN AVSNADLMDF
KNLLDHLEEK MPLEDEVV@O QVLSEPNEEA GAALS@LPEV
OOWTGEVSOA QRDGGALGRG PWDSSDRSAL LKSKLRALLT
AORSLRRSSC FGGRMDRIGA QSGLGCNSFR Y

While ANF has only two predicted Hyp-glycosylation sites, it has a very strong motif, AALSPSPEVPP (amino acids 72 to 82 of SEQ ID NO:7)—rich in clustered Pro and has lots of Ala Ser Val.

### Collagen Type I Alpha (NP000079.1)

(SEQ ID NO: 8)

MFSFVDLRLL LLLAATALLT HGQEEGQVEG QDEDIPOITC
VQNGLRYHDR DVWKPEPCRI CVCDNGKVLC DDVICDETKN
CPGAEVPEGE CCPVCPDGSE SOTDQETTGV EGPKGDTGOR
GPRGOAGOOG RDGIPGQPGL PG@OG@OG@O G@OGLGGNFA
PQLSYGYDEK STGGISV#GO MGOSGORGLP G@OGA#GPQG
FQGOOGEPGE PGASGPMGPR GOOG@OGKNG DDGEAGKPGR
PGERGOOGPQ GARGLPGTAG LPGMKGHRGF SGLDGAKGDA
GOAGPKGEPG SOGENGAOGQ MGPRGLPGER GRPGA#G#AG
ARGNDGATGA AG@OGOTGOA G@OGFPGAVG AKGEAGPQGP
RGSEGPQGVR GEPG@OGOAG AAG#AGNPGA DGQPGAKGAN
GA#GIAGAOG FPGARGOSGP QGOGG@OG@K GNSGEPGAOG
SKGDTGAKGE PGOVGVQGOO G#AGEEGKRG ARGEPGOTGL
PG@OGERGGO GSRGFPGADG VAGOKGOQAGE RGS#G#AGOK
GSOGEAGRPG EAGLPGAKGL TGSOGS#GOD GKTG@OGOAG
QDGRPG@OG@ OGARGQAGVM GFPGPKGAAG EOGKAGERGV
PG@OGAVGOA GKDGEAGAQG OOG#AGOAGE RGEQGOAGSO
GFQGLPG#AG @OGEAGKPGE QGVOGDLGA# G#SGARGERG
FPGERGVQGP PG#AGPRGAN GAOGNDGAKG DAGA#GA#GS
QGAOGLQGMP GERGAAGLPG PKGDRGDAGP KGADGSPGKD
GVRGLTGPIG OOG#AGAOGD GESGPSG#A GOTGARGAOG
DRGEPGOOGO AGFAG@OGAD
GQPGAKGEPG DAGAKGDAC@ OGOAGOAG@O GOIGNVGAOG
AKGARGSAG@ OGATGFPGAA GRVG@OGOSG NAG@OGOOGO
AGKEGGKGPR GETGOAGRPG EVG@OG@OGO AGEKGSOGAD
GOAGAOGT@G OQGIAGQRGV
VGLPGQRGER GFPGLPG#SG EPGKQGOSGA SGERGOOGOM
GOOGLAG@OG ESGREGA#AA
EGSOGRDGSO GAKGDRGETG OAG@OGAOGA OGA#GOVGOA
GKSGDRGETG OAGOAGOVGO VGARGOAGOQ GPRGDKGETG
EQGDRGIKGH RGFSGLQGOO G@OGSOGEQG OSGASG@AGO
RGOOGSAGAO GKDGLNGLPG OIGOOGPRGR TGDAGOVG@O
G@OG@OG@OG @OSAGFDFSF LPQPPQEKAH DGGRYYRADD
ANVVRDRDLE VDTTLKSLSQ QIENIRSPEG SRKNPARTCR
DLKMCHSDWK SGEYWIDPNQ GCNLDAIKVF CNMETGETCV
YPTQPSVAQK NWYISKNPKD KRHVWFGESM TDGFQFEYGG
QGSDPADVAI QLTFLRLMST EASQNITYHC KNSVAYMDQQ
TGNLKKALLL KGSNEIEIRA EGNSRFTYSV TVDGCTSHTG
AWGKTVIEYK TTKSSRLPII DVAOLDVGAO DQEFGFDVGP
VCFL

### Colony Stimulating Factor (NP000749.2)

(SEQ ID NO: 9)

MWLQSLLLLG TVACSISA#A RS#S#STQPW EHVNAIQEAR
RLLNLSRDTA AEMNETVEVI SEMFDLQEPT CLQTRLELYK
QGLRGSLTKL KGPLTMMASH YKQHCPPT@E TSCATQIITF
ESFKENLKDF LLVIPFDCWE PVQE

Endo-1,4-b-D-Glucanase, Ziegler et al, Molecular Breeding 6:37-46 (2000).

(SEQ ID NO: 10)

MPRALRRVPGSRVMLRVGVVVAVLALVAALANLAV#RPARAAGGYWHTSG
REILDANNVOVRIAGINWFGFETCNYVVHGLWSRDYRSMLDQIKSLGYNT
IRLPYSDDILKPGTMPNSINFYQMNQDLQGLTSLQVMDKIVAYAGQIGLR
IILDRHRPDCSGQSALWYTSSVSEATWISDLQALAQRYKGNPTVVGFDLH
NEPHDPACWGCGDPSIDWRLAAERAGNAVLSVNPNLLIFVEGVQSYNGDS
YWWGGNLQGAGQYPVVLNVPNRLVYSAHDYATSVYPQTWFSDPTFPNNMP
GIWNKNWGYLFNQNIAOVWLGEFGTTLQSTTDQTWLKTLVQYLRPTAQYG
ADSFQWTFWSWNPDSGDTGGILKDDWQTVDTVKDGYLAOIKSSIFDPVGA
SAS#SSQPS#SVS#S#S#S#SASRT@T@T@T@TAS#T@TLT#TAT@T@TA
SOTOSOTAASGARCTASYQVNSDWGNGFTVTVAVTNSGSVATKTWTVSWT
FGGNQTITNSWNAAVTQNGQSVTARNMSYNNVIQPGQNTTFGFQASYTGS
NAAOTVACAAS

### Fibrosin 1 (NM002245.1)

(SEQ ID NO: 11)

MHVRVAYMIL RHQEKMKGDS HKLDFRNDLL PCLPGOYGAL
POGQELSHPA SLFTATGAVH AAANPFTAA# GAHGPFLSOS
THIDPFGRPT SFASLAALSN GAFGGLGSOT FNSGAVFAQK
ES#GA@OAFA SOODPWGRLH RSOLTFPAWV RPOEAARTOG
SDKERPVERR EPSITKEEKD RDLPFSRPQL RVS#AT@KAR
AGEEGORPTK ESVRVKEERK EEAAAAAAAA AAAAAAAAAA
ATGPQGLHLL FERPRP@OFL G#S#ODRCAG FLEPTWLAA@
ORLARPORFY EAGEELTGOG AVAAARLYGL EOAHPLLYSR
LA@@@@@AAA #GTOHLLSKT @OGALLGA@@ @LV#A#RPSS
@ORG#GOAPA DR

### Human Granulocyte Macrophage Colony Stimulating Factor (AAA98768)

(SEQ ID NO: 12)

mwlqsllllg tvacsisa#a rs#s#stqpw ehvnaiqear
rllnlsrdta aemnetvevi semfdlqept clqtrlelyk
qglrgsltkl kgpltmmash ykqhcppt@e tscatqiitf
esfkenlkdf llvipfdcwe pvqe

### Immunoglobin AM2 (AAH65733.1)

(SEQ ID NO: 13)

MDWTWRILFL AAAATGVQSQ VQLVQSGAEV KKTGASVKVS
CKASGYSISD NYIHWVRQAO GQGLEWMAWI RPQNGGTVSA
EKFQGRVTIT IDTSLNTAYM ELTSLKSDDT ALYYCARGHS
DWSSYYFDYW GQGTLVTVSS AS#TS@KVFP LSLDSTOQDG
NVVVACLVQG FFPQEPLSVT WSESGQNVTA RNFPOSQDAS
GDLYTTSSQL TLPATQCPDG KSVTCHVKHY TNPSQDVTVO
CPV@@@OOCC HPRLSLHRPA LEDLLLGSEA NLTCTLTGLR
DASGATFTWT PSSGKSAVQG OOERDLCGCY SVSSVLPGCA
QPWNHGETFT CTAAHPELKT OLTANITKSG NTFRPEVHLL
P@OSEELALN ELVTLTCLAR GFSPKDVLVR WLQGSQELPR
EKYLTWASRQ EPSQGTTTFA VTSILRVAAE DWKKGDTFSC
MVGHEALPLA FTQKTIDRLA GKPTHVNVSV VMAEVDGTCY

### Immunoglobin Heavy Constant Delta (AAH63384.1)

(SEQ ID NO: 14)

MGLLHKNMKH LWFFLLLVAA ORWVLSQVQL QESGOGLVKP
SGTLSLTCAV SGGSISSSNW WSWVRQPOGK GLEWIGEIYH
SGSTNYNPSL KSRVTISVDK SKNQFSLKLS SVTAADTAVY
YCASLGDIYY YGMDVWGQGT TVTVSSA#TK AODVFPIISG
CRHPKDNSOV VLACLITGYH PTSVTVTWYM GTQSQPQRTF
PEIQRRDSYY MTSSQLSTOL QQWRQGEYKC VVQHTASKSK
KEIFRWPESO KAQASSV#TA QPQAEGSLAK ATTA#ATTRN
TGRGGEEKKK EKEKEEQEER ETKTPECPSH TQPLGVYLLT
OAVQDLWLRD KATFTCFVVG SDLKDAHLTW EVAGKVOTGG
VEEGLLERHS NGSQSQHSRL TLPRSLWNAG TSITCTLNHP
SLPPQRLMAL REOAAQAOVK LSLNLLASSD POEAASWLLC
EVSGFSOONI LLMWLEDQRE VNTSGFAOAR POOQPGSTTF
WAWSVLRVOA @OS#QPATYT CVVSHEDSRT LLNASRSLEV
SYLAMTPLIP QSKDENSDDY TTFDDVGSLW TTLSTFVALF ILTLLYSGIV
TFIKVK

Interleukin 11 (nm000641.1)

(SEQ ID NO: 15)

MNCVCRLVLV VLSLWPDTAV AOG@@@GOOR VS#DPRAELD
STVLLTRSLL ADTRQLAAQL RDKFPADGDH NLDSLPTLAM
SAGALGALQL PGVLTRLRAD LLSYLRHVQW LRRAGGSSLK
TLEPELGTLQ ARLDRLLRRL QLLMSRLALP QPOODPOA@O
LA@OSSAWGG IRAAHAILGG LHLTLDWAVR GLLLLKTRL

The same prolines are predicted to be Hyp-glycosylation sites or Pro-hydroxylation sites regardless of whether one inputs the entire sequence or just the mature sequence.

### Interleukin 13 (NP002179.1)

(SEQ ID NO: 16)

MALLLTTVIA LTCLGGFAS# G#V@OSTALR ELIEELVNIT
QNQKAOLCNG SMVWSINLTA GMYCAALESL INVSGCSAIE
KTQRMLSGFC PHKVSAGQFS SLHVRDTKIE VAQFVKDLLL
ELKKLFREGR FN

The same prolines are predicted to be Hyp-glycosylation sites or Pro-hydroxylation sites regardless of whether one inputs the entire sequence or just the mature sequence.

### Mucin 1 (P18941)

(SEQ ID NO: 17)

MTOGTQSOFF LLLLLTVLTV VTGSGHASST OGGEKETSAT
QRSSV#SSTE KNAVSMTSSV LSSHS#GSGS STTQGQDVTL
A #ATE #ASGS AATWGQDVTS VOVTPPALGS TT @OAHDVTS
AODNKPA #GS TAO*A)OAHGVTS A #DTRPAOGS TA @OAHGVTS
A #DTRPA #GS TA @OAHGVTS A #DTRPA #GS TA @OAHGVTS
A #DTRPA #GS TA @OAHGVTS A #DTRPAOGS TA @OAHGVTS
A #DTRPA #GS TA @OAHGVTS A #DTRPAOGS TA @OAHGVTS
A #DTRPA #GS TA @OAHGVTS A #DTRPAOGS TA @OAHGVTS
A #DTRPA #GS TA @OAHGVTS A #DTRPAOGS TA @OAHGVTS
A #DTRPA #GS TA @OAHGVTS A #DTRPAOGS TA @OAHGVTS
A #DTRPA #GS TA @OAHGVTS A #DTRPAOGS TA @OAHGVTS
A #DTRPA #GS TA @OAHGVTS A #DTRPAOGS TA @OAHGVTS
A #DTRPA #GS TA @OAHGVTS A #DTRPAOGS TA @OAHGVTS
A #DTRPA #GS TA @OAHGVTS A #DTRPAOGS TA @OAHGVTS
A #DTRPA #GS TA @OAHGVTS A #DTRPAOGS TA @OAHGVTS
A #DTRPA #GS TA @OAHGVTS A #DTRPAOGS TA @OAHGVTS
A #DTRPA #GS TA @OAHGVTS A #DTRPAOGS TA @OAHGVTS
A #DTRPA #GS TA @OAHGVTS A #DTRPAOGS TA @OAHGVTS
A #DTRPA #GS TA @OAHGVTS A #DTRPAOGS TA @OAHGVTS
A #DTRPA #GS TA @OAHGVTS A #DTRPAOGS TA @OAHGVTS
A #DTRPA #GS TA @OAHGVTS A #DTRPAOGS TA @OAEGVTS
A #DTRPA #GS TA @OAHGVTS A #DTRPAOGS TA @OAHGVTS
A #DTRPA #GS TA @OAHGVTS A #DTRPAOGS TA @OAHGVTS
A #DTRPA #GS TA @OAHGVTS A #DNRPALGS TA@OVHNVTS
ASGSASGSAS TLVHNGTSAR ATTT #ASKST OFSIPSHHSD
TOTTLASHST KTDASSTHHS SV @OLTSSNH STS #QLSTGV
SFFFLSFHIS NLQFNSSLED PSTDYYQELQ RDISEMFLQI
YKQGGFLGLS NIKFRPGSVV VQLTLAFREG TINVHDVETQ
FNQYKTEAAS RYNLTISDVS VSDVOFPFSA QSGAGVOGWG
IALLVLVCVL VALAIVYLIA LAVCQCRRKN YGQLDIFPAR
DTYHPMSEYP TYHTEGRYV@ OSSTDRSOYE KVSAGNGGSS
LSYTNPAVAA ASANL

### Mucin 7 Salivary (NP689504.1)

(SEQ ID NO: 18)

MKTLPLFVCI CALSACFSFS EGRERDEELR HRRHHHQS@K
SHFELPHYPG LLAHQKPFIR KSYKCLHKRC RPKLPOSONN
POKFPNPHQP OKHPDKNSSV VNPTLVATTQ IPSVTFPSAS
TKITTLPNVT FLPQNATTIS SRENVNTSSS VATLAOVKSO
AOQDTTAA@O T#SATT#A@O SSSA@OETTA A@OT#SATTQ
A@OSSSA@OE TTAA@OT@OA TTOAOOSSSA @OETTAA@OT
#SATT@A#LS SSA@OETTAV @OT#SATTLD PSSASA@OET
TAA@OT#SAT T#A@OSS#A# QETTAAOITT #NSS#TTLAO
DTSETSAA#T HQTITSVTTQ TTTTKQPTSA OGQNKISRFL
LYMKNLLNRI IDDMVEQ

Other mucins are expected, when expressed and secreted in plants, to contain Hyp-glycomodules, too.

### C1 Orf32 Protein (NP955383.1)

(SEQ ID NO: 20)

MDRVLLRWIS LFWLTAMVEG LQVTVPDKKK VAMLFQPTVL
RCHFSTSSHQ PAVVQWKFKS YCQDRMGESL GMSSTRAQSL
SKRNLEWDPY LDCLDSRRTV RVVASKQGST VTLGDFYRGR
EITIVHDADL QIGKLMWGDS GLYYCIITTP DDLEGKNEDS
VELLVLGRTG LLADLLPSFA VEIMPEWVFV GLVLLGVFLF
FVLVGICWCQ CCPHSCCCYV RCPCCPDSCC CPQALYEAGK
AAKAGYPOSV SGV#G#YSIP SVOLGGAPSS GMLMDKPHO@
OLAOSDSTGG SHSVRKGYRI QADKERDSMK VLYYVEKELA
QFDPARRMRG RYNNTISELS SLHEEDSNFR QSFHQMRSKQ
FPVSGDLESN PDYWSGVMGG SSGASRGPSA MEYNKEDRES
FRHSQPRSKS EMLSRKNFAT GVPAVSMDEL AAFADSYGQR
PRRADGNSHE ARGGSRFERS ESRAHSGFYQ DDSLEEYYGQ
RSRSREPLTD ADRGWAFSPA RRRPAEDAHL PRLVSRTPGT
APKYDHSYLG SARERQARPE GASRGGSLET #SKRSAQLGP
RSASYYAWSO #GTYKAGSSQ DDQEDASDDA LPPYSELELT
RGPSYRGRDL PYHSNSEKKR KKEPAKKTND FPTRMSLVV

C1-orf32, with five predicted Glyco-Hyp, has its proline-rich region in the middle of the protein and the Pro's are somewhat spread out. In contrast, while CSF has just two predicted Glyco-Hyp, it has a very strong hydroxylation/arabinogalactosylation region right at the N-terminus of the mature sequence, SPSPST . . . (AAs 22 to 27 of SEQ ID NO: 9). This sequence resembles those that we deliberately add to the end of hGH, interferon etc to introduce hydroxylation/glycosylation.
It should be noted that the program may have a false negative at Pro-268 of C1-orf32. The region 245-285 has quite a bit of Pro (12 of 40 residues) which means it probably has fairly rigid and extended stretches and that region has an abundance of amino acids common in HRGPs.
Also, in the subsequence predicted above to be HO@ OLAO (AAs 278-284), it is likely that third proline will also be arabinosylated, and that the fourth proline will also be arabinogalactosylated.
II. Examples of Non-Plant Proteins that MIGHT be Partially Hydroxylated at the Bolded, Underlined Proline Residues.

The amino acids immediately surrounding these Pro's favor hydroxylation (A, S, T, V, P) but the overall environment (21 amino acid window) is not particularly not rich in A, S, T, V, or P and the target Pros are quite isolated from one another . . . or they occur within folded parts of the protein and unlikely to be exposed to the post-translational machinery.

The environment is not considered rich if the 21 amino acid window (not counting the target residue on which it is centered) is less than 10% Pro, less than 10% A, less than 10% S, less than 10% T, and less than 10% V.

A protein is considered likely to be folded if it contains an even number of Cys residues, since these are likely to be paired off in disulfide bonds, and the disulfide bonds are likely to stabilize a folded conformation.

It is also considered likely to be folded if it has a low content of Hyp and Pro. Pro (and Hyp) rigidize the polypeptide chain, whereas other amino acids are flexible and allow the chain to fold.

It may therefore be advantageous to 1) mutate one or more non-proline amino acids to proline, at positions predicted to then be Hyp-glycosylation sites, 2) mutate one or more amino acids in the vicinity of a proline so as to increase the Hyp-score of that proline or the degree of glycosylation predicted to occur if that proline is hydroxylated, and/or 3) add a Hyp-glycomodule to one or both ends of the protein.

Acidic Mammalian Chitinase (aag60019.1)

(SEQ ID NO: 19)

MTKLILLTGL VLILNLQLGS AYQLTCYFTN WAQYRPGLGR
FMPDNIDPCL CTHLIYAFAG RQNNEITTIE WNDVTLYQAF
NGLKNKNSQL KTLLAIGGWN FGTAPFTAMV STPENRQTFI
TSVIKFLRQY EFDGLDFDWE YPGSRGSPPQ DKELFTVLVQ
EMREAFEQEA KQINKPRLMV TAAVAAGISN IQSGYEIPQL
SQYLDYIHVM TYDLHGSWEG YTGENSPLYK YPTDTGSNAY
LNVDYVMNYW KDNGAPAEKL IVGFPTYGHN FILSNPSNTG
IGA#TSGAG# AGPYAKESGI WAYYEICTFL KNGATQGWDA
PQEVPYAYQG NVWVGYDNIK SFDIKAQWLK HNKFGGAMVW
AIDLDDFTGT FCNQGKFPLI STLKKALGLQ SASCTA#AQP
IEPITAA#SG SGNGSGSSSS GGSSGGSGFC AVRANGLYPV
ANNRNAFWHC VNGVTYQQNC QAGLVFDTSC DCCNWA

In group II because of the high number of cysteines, including several close to the predicted sites.

### Calcitonin (NM001741.1)

(SEQ ID NO: 21)

MGFQKFSPFL ALSILVLLQA GSLHAAPFRS ALESS#ADPA
TLSEDEARLL LAALVQDYVQ MKASELEQEQ EREGSSLDSP
RSKRCGNLST CMLGTYTQDF NKFHTFPQTA IGVGAPGKKR
DMSSDLERDH RPHVSMPQNA N_

In group II, not III, despite having only one predicted Hyp-glycosylation site, since Ser, Ala and Pro nearby. The Calcitonin sequence is near a terminus and is not sandwiched between Cys residues. The motif SSPADP (AAs 34-39) has loosely clustered Pro and Ser plus Ala make up half the amino acids in the motif.

### Erythropoietin (NM000799.1)

(SEQ ID NO: 22)

MGVHECPAWL WLLLSLLSLP LGLPVLGA@O RLICDSRVLE
RYLLEAKEAE NITTGCAEHC SLNENITVPD TKVNFYAWKR
MEVGQQAVEV WQGLALLSEA VLRGQALLVN SSQPWEPLQL
HVDKAVSGLR SLTTLLRALR AQKEAIS#OD AASAAPLRTI
TADTFRKLFR VYSNFLRGKL KLYTGEACRT GDR

The same prolines are predicted to be Hyp-glycosylation sites or Pro-hydroxylation sites regardless of whether one inputs the entire sequence or just the mature sequence.

### Immunoglobin Lambda Constant 2 (AAH73762.1)

(SEQ ID NO: 24)

MAWTLLLLVL LSHCTGSLSQ PVLTQPSSHS ASSGASVRLT
CMLSSGFSVG DFWIRWYQQK PGNPPRYLLY YHSDSNKGQG
SGVPSRFSGS NDASANAGIL RISGLQPEDE ADYYCGAWHS
NSKTVVFGGG TRLTVLGQPK AA#SVTLFPO SSEELQANKA
TLVCLISDFY PGAVTVAWKA DSSOVKAGVE TTT#SKQSNN
KYAASSYLSL TPEQWKSHRS YSCQVTHEGS TVEKTVA#TE
CS

### Nodal Related Protein (AAH33585)

(SEQ ID NO: 26)

MHAHCLPFLL HAWWALLQAG AATVATALLR TRGQPSS#S#
LAYMLSLYRD PLPRADIIRS LQAEDVAVDG QNWTFAFDFS
FLSQQEDLAW AELRLQLSSP VDLPTEGSLA IEIFHQPKPD
TEQASDSCLE RFQMDLFTVT LSQVTFSLGS MVLEVTRPLS
KWLKRPGALE KQMSRVAGEC WPRPPT@PAT NVLLMLYSNL
SQEQRQLGGS TLLWEAESSW RAQEGQLSWE WGKRHRRHHL
PDRSQLCRKV KFQVDFNLIG WGSWIIYPKQ YNAYRCEGEC
PNPVGEEFHP TNHAYIQSLL KRYQPHRVPS TCCAPVKTKP
LSMLYVDNGR VLLDHHKDMI VEECGCL

### Platelet Glycoprotein VI (BAB12247.1)

(SEQ ID NO: 27)

MS#S#TALFC LGLCLGRVPA QSG#LPKPSL QALPSSLVPL
EKPVTLRCQG PPGVDLYRLE KLSSSRYQDQ AVLFIPAMKR
SLAGRYRCSY QNGSLWSLPS DQLELVATGV FAKPSLSAQP
G#AVSSGGDV TLQCQTRYGF DQFALYKEGD PAPYKNPERW
YRASFPIITV TAAHSGTYRC YSFSSRDPYL WSAPSDPLEL
VVTGTSVTPS RLPTE@PSSV AEFSEATAEL TVSFTNKVFT
TETSRSITTS @KESDS#AGE SCPPVLHQGQ PGPDMPRGCD
PNNPGGVSGR GLAQPEEAPA AQGQGCAEAA SA#AA@OADP
EITRGSGWRP TGCSQPRVMF MTAEPQARSY PREGSWHGRR
LKDWRVWSVE AGGQRLQLWK RGHAASSWCS IREPFGQCLS
VCLPLCLRAP SIWDGRNLWR PHPPPCTLWM TWYPGWTTYW
PLSSTSLIWA PDGSLRFPAL RVDSVPSSVQ NPPVLPFGPL
CSCLVFPRNS HPHSISHCGL TNLLSSLRTG LAGSLGMSFI
FLSVKLARCP LPFTLENKIS LCNMVKPHLY QQNKKTQKLA
RCGGASLYSQ QLGGLRWENG LSLGGRGCSE LRSHHCTLAR
VTKPDLVSKN TGMNMSITLI

### Carcinoembryonic Antigen Related Cell Adhesion Molecule (NP001703.2)

(SEQ ID NO: 28)

MGHLSAPLHR VRVPWQGLLL TASLLTFWNP PTTAQLTTES
MPFNVAEGKE VLLLVHNLPQ QLFGYSWYKG ERVDGNRQIV
GYAIGTQQAT @GOANSGRET IYPNASLLIQ NVTQNDTGFY
TLQVIKSDLV NEEATGQFHV YPELPKPSIS SNNSNPVEDK
DAVAFTCEPE TQDTTYLWWI NNQSLPVSOR LQLSNGNRTL
TLLSVTRNDT GOYECEIQNP VSANRSDPVT LNVTYGODTO
TISOSDTYYR PGANLSLSCY AASNP#AQYS WLINGTFQQS
TQELFIPNIT VNNSGSYTCH ANNSVTGCNR TTVKTIIVTE
LSOVVAKPQI KASKTTVTGD KDSVNLTCST NDTGISIRWF
FKNQSLPSSE RMKLSQGNTT LSINPVKRED AGTYWCEVFN
PISKNQSDPI MLNVNYNALP QENGLSOGAI AGIVIGVVAL
VALIAVALAC FLHFGKTGRA SDQRDLTEHK PSVSNETQDH
SNDPONKMNE VTYSTLNFEA QQPTQPTSAS #SLTATEIIY
SEVKKQ

Add an arabinogalactosylation site at residue 513 by mutating L to Pro; Add an arabinogalactosylation site at residue 506 by mutating Q-505 to S or A. The mutations are for regions of the protein that are HRGP-like (High Ser, Ala, Thr, and preexisting Pro) and therefore more likely to be modified after a little tweaking.

### Immunoglobin Mu (CAA 34971.1)

(SEQ ID NO: 38)

MDWTWRFLFV VAAATGVQSQ VQLVQSGAEV KKPGSSVKVS
CKASGGTFSS YAISWVRQAO GQGLEWMGGI IPIFGTANYA
QKFQGRVTIT ADESTSTAYM ELSSLRSEDT AVYYCAKTGI
LGPYSSGWYP NSDYYYYGMD VWGQGTTVTV SSGSASA#TL
FPLVSCENSO SDTSSVAVGC LAQDFLPDSI TFSWKYKNNS
DISSTRGFPS VLRGGKYAAT SQVLLPSKDV MQGTDEHVVC
KVQHPNGNKE KNVOLPVIAE LPOKVSVFVP ORDGFFGNPR
SKSKLICQAT GFSORQIQVS WLREGKQVGS GVTTDQVQAE
AKESGOTTYK VTSTLTIKES DWLSQSMFTC RVDHRGLTFQ
QNASSMCVPD QDTAIRVFAI POSFASIFLT KSTKLTCLVT
DLTTYDSVTI SWTRQNGEAV KTHTNISESH PNATFSAVGE
ASICEDDWNS GERFTCTVTH TDLPS#LKQT ISRPKGVALH
RPDVYLLPOA REQLNLRESA TITCLVTGFS OADVFVQWMQ
RGQPLSOEKY VTSA#MPEOQ APGRYFAHSI LTVSEEEWNT
GETYTCVVAH EALPNRVTER TVDKSTEGEV SADEEGFENL
WATASTFIVL FLLSLFYSTT VTLFKVK

This protein has three predicted AraGal-Hyp sites. The third of these is the most likely to be accessible to the enzymes because it is in a Pro-rich stretch SA#MPEPQAP (amino acids 533-542 of SEQ ID NO:38).
You may add arabinogalactosylation by mutating T 619 to Pro, Val 621 to Ser, Thr 622 to Pro. I suggest these mutations because they occur near an end of the protein.
III. Examples of Non-Plant Proteins that are Unlikely to be Hydroxylated at Proline.

The proteins of this category are likely to require modification in order to exhibit Hyp-glycosylation. It may therefore be advantageous to 1) mutate one or more non-proline amino acids to proline, at positions predicted to then be Hyp-glycosylation sites, 2) mutate one or more amino acids in the vicinity of a proline so as to increase the Hyp-score of that proline or the degree of glycosylation predicted to occur if that proline is hydroxylated, and/or 3) add a Hyp-glycomodule to one or both ends of the protein.

The addition Hyp-glycomodule strategy can be used with any of the proteins. However, for some of the proteins in this category, we also suggest below some specific substitutions which will create predicted arabinogalactosylated Hyp-glycosylation sites within those proteins. This could be done, without undue experimentation, for all of the proteins. Likewise, predicted arabinosylated Hyp-glycosylation sites can be created. Of course, finding mutations which will not also adversely affect biological activity is more difficult. See the discussion of mutational strategies, above.

### Ghrelin (NP057446.1)

(SEQ ID NO: 23)

MPSPGTVCSL LLLGMLWLDL AMAGSSFLSP EHQRVQQRKE
SKKPPAKLQP RALAGWLRPE DGGQAEGAED ELEVRFNAPF
DVGIKLSGVQ YQQHSQALGK FLQDILWEEA KEAOADK

Note that while the program, if input the whole sequence, would predict Pro-4 to be arbinogalactosylated, it is part of the signal peptide, and hence removed before glycosylation occurs.
We suggest mutating Asp-115 to Pro to create a predicted AraGal-Hyp site.
Interleukin 2 (np000577.2)

(SEQ ID NO: 25)

MYRMQLLSCI ALSLALVTNS A#TSSSTKKT QLQLEHLLLD
LQMILNGINN YKNPKLTRML TFKFYMPKKA TELKHLQCLE
EELKPLEEVL NLAQSKNFHL RPRDLISNIN VIVLELKGSE
TTFMCEYADE TATIVEFLNR WITFCQSIIS TLT

Just one predicted Hyp-glycosylation site.
May mutate Ser-24 to Pro and/or Ser-26 to Pro.

### Coagulation Factor (AAH30229)

(SEQ ID NO: 29)

MPAWGALFLL WATAEATKDC PSOCTCRALE TMGLWVDCRG
HGLTALPALP ARTRHLLLAN NSLQSV@OGA FDHLPQLQTL
DVTQNPWHCD CSLTYLRLWL EDRTOEALLQ VRCAS#SLAA
HGPLGRLTGY QLGSCGWQLQ ASWVRPGVLW DVALVAVAAL
GLALLAGLLC ATTEALD

While coagulation factor has predicted Hyp-glycosylation sites, they aren't in Pro-rich regions, and hence are not likely to have an extended conformation (random coil, extended strand, polyproline helix).

Add Arabinogalactosylation sites at residues 47 and 50 by mutating L residues 46 and 49 to A or S. The mutations are for regions of the protein that are HRGP-like (High Ser, Ala, Thr, and preexisting Pro) and therefore more likely to be modified after a little tweaking.

### Fibroblast Growth Factor 1 (NM000800.2)

(SEQ ID NO: 30)

MAEGEITTFT ALTEKFNLPP GNYKKPKLLY CSNGGHFLRI
LPDGTVDGTR DRSDQHIQLQ LSAESVGEVY IKSTETGQYL
AMDTDGLLYG SQTPNEECLF LERLEENHYN TYISKKHAEK
NWFVGLKKNG SCKRGPRTHY GQKAILFLPL PVSSD

Add arabinogalactosylation sites at residues 149 and 151 by mutating L residues 148 and 150 to A or S

### Fibroblast Growth Factor 6 (NP066276.2)

(SEQ ID NO: 31)

MALGQKLFIT MSRGAGRLQG TLWALVFLGI LVGMVVPSPA
GTRANNTLLD SRGWGTLLSR SRAGLAGEIA GVNWESGYLV
GIKRQRRLYC NVGIGFHLQV LPDGRISGTH EENPYSLLEI
STVERGVVSL FGVRSALFVA MNSKGRLYAT PSFQEECKFR
ETLLPNNYNA YESDLYQGTY IALSKYGRVK RGSKVSOIMT
VTHFLPRI

If this sequence is considered in its entirety, Pro-37 is predicted to become arabinogalactosylated Hyp (#). However, that fails to take into account the fact that Pro-37 is part of the signal sequence. Another nominally predicted # site is at Pro-39. However, that fails to take into account that signal peptide residues are within the windows used in the predictive methods. If only the sequence of the mature protein is input, neither Pro-37 nor Pro-39 are predicted to be hydroxylated (and hence, there is no Hyp to be glycosylated).
The program still predicts that Pro-196 is hydroxylated (as shown above), but it is not thereby predicted to be glycosylated.

Add arabinogalactosylation sites at residues 197, 199 and 201 mutating 1198 to A or S and M 199 and V 201 both to P

### Fibroblast Growth Factor 7 (NP002000.1)

(SEQ ID NO: 32)

MHKWILTWIL PTLLYRSCFH IICLVGTISL ACNDMTPEQM
ATNVNCSSPE RHTRSYDYME GGDIRVRRLF CRTQWYLRID
KRGKVKGTQE MKNNYNIMEI RTVAVGIVAI KGVESEFYLA
MNKEGKLYAK KECNEDCNFK ELILENHYNT YASAKWTHNG
GEMFVALNQK GIPVRGKKTK KEQKTAHFLP MAIT

This protein presents us with the interesting opportunity for mutating a parental protein to facilitate secretion in plant cells and simultaneously produced an antagonist. FGF-7 binds heparin through the interaction of positively charged Lys residues with the negatively charged heparin. See Wong and Burgess, “FGF2-Heparin Co-crystal Complex-assisted Design of Mutants FGF1 and FGF7 with Predictable Heparin Affinities,” J. Bio. Chem., 273(29), 18617-18622 (1998).
Addition of bulky groups like arabinosides or, worse, negatively charged arabinogalactan will likely interfere binding of negatively-charged heparin by the positively charged Lys residues near the C-terminal.
So if I wanted to make an antagonist I suggest mutating 1172 to S, A or P and K 170 to P.

### Growth Hormone 1 (NM000506.2)

(SEQ ID NO: 33)

MATGSRTSLL LAFGLLCLPW LQEGSAFPTI PLSRLFDNAM
LRAHRLHQLA FDTYQEFEEA YIPKEQKYSF LQNPQTSLCF
SESIPTOSNR EETQQKSNLE LLRISLLLIQ SWLEPVQFLR
SVFANSLVYG ASDSNVYDLL KDLEEGIQTL MGRLEDGSOR
TGQIFKQTYS KFDTNSHNDD ALLKNYGLLY CFRKDMDKVE
TFLRIVQCRS VEGSCGF

Add arabinosylation site at residues 30-31 by mutating I-30 to Ser or Ala.

### Growth Hormone 2 (NM022557.2)

(SEQ ID NO: 34)

MAAGSRTSLL LAFGLLCLSW LQEGSAFPTI PLSRLFDNAM
LRARRLYQLA YDTYQEFEEA YILKEQKYSF LQNPQTSLCF
SESIPTOSNR VKTQQKSNLE LLRISLLLIQ SWLEPVQLLR
SVFANSLVYG ASDSNVYRHL KDLEEGIQTL MWVRVAOGIP
NPGAOLASRD WGEKHCCPLF SSQALTQENS OYSSFPLVNP
OGLSLQPGGE GGKWMNERGR EQCPSAWPLL LFLHFAEAGR
WQPPDWADLQ SVLQQV

Add arabinosylation site at residues 30-31 by mutating 1-30 to Ser or Ala

### Green Fluorescent Protein (Enhanced) (AAB02574.1)

(SEQ ID NO: 35)

MVSKGEELFT GVVPILVELD GDVNGHKFSV SGEGEGDATY
GKLTLKFICT TGKLPVPWPT LVTTLTYGVQ CFSRYPDHMK
QHDFFKSAMP EGYVQERTIF FKDDGNYKTR AEVKFEGDTL
VNRIELKGID FKEDGNILGH KLEYNYNSHN VYIMADKQKN
GIKVNFKIRH NIEDGSVQLA DHYQQNTPIG DGPVLLPDNH
YLSTQSALSK DPNEKRDHMV LLEFVTAAGI TLGMDELYK

Add arabinogalactosylation by mutating Val 11 to Pro and Val 12 to Ser. The N-terminus is not crucial for function so these mutations may be tolerated.
The difference between enhanced GFP and ordinary GFP is that the former contains two amino acid substitutions in the vicinity of the chromophore (Phe-64 to Leu, Ser-65 to Thr).

### Human Protein C

(SEQ ID NO: 36)

MWQLTSLLLF VATWGISGTP APLDSVFSSS ERAHQVLRIR
KRANSFLEEL RHSSLERECI EEICDFEEAK EIFQNVDDTL
AFWSKHVDGD QCLVLPLEHP CASLCCGHGT CIDGIGSFSC
DCRSGWEGRF CQREVSFLNC SLDNGGCTHY CLEEVGWRRC
SCAPGYKLGD DLLQCHPAVK FPCGRPWKRM EKKRSHLKRD
TEDQEDQVDP RLIDGKMTRR GDSPWQVVLL DSKKKLACGA
VLIHPSWVLT AAHCMDESKK LLVRLGEYDL RRWEKWELDL
DIKEVFVHPN YSKSTTDNDI ALLHLAQPAT LSQTIVPICL
PDSGLAEREL NQAGQETLVT GWGYHSSREK EAKRNRTFVL
NFIKIPVVPH NECSEVMSNM VSENMLCAGI LGDRQDACEG
DSGGOMVASF HGTWFLVGLV SWGEGCGLLH NYGVYTKVSR
YLDWIHGHIR DKEAOQKSWA P

Here, Pro-20 and -22 would be predicted to be hydroxylated were they not part of the signal sequence.

Add arabinogalactosylation sites by mutating W-359 to P, Q-356 to A and K-357 to P

### Human Serum Albumin

(SEQ ID NO: 37)

MKWVTFISLL FLFSSAYSRG VFRRDAHKSE VAHRFKDLGE
ENFKALVLIA FAQYLQQCPF EDHVKLVNEV TEFAKTCVAD
ESAENCDKSL HTLFGDKLCT VATLRETYGE MADCCAKQEP
ERNECFLQHK DDNPNLPRLV RPEVDVMCTA FHDNEETFLK
KYLYEIARRH PYFYAPELLF FAKRYKAAFT ECCQAADKAA
CLLPKLDELR DEGKASSAKQ RLKCASLQKF GERAFKAWAV
ARLSQRFPKA EFAEVSKLVT DLTKVHTECC HGDLLECADD
RADLAKYICE NQDSISSKLK ECCEKPLLEK SHCIAEVEND
EMPADLPSLA ADFVESKDVC KNYAEAKDVF LGMFLYEYAR
RHPDYSVVLL LRLAKTYETT LEKCCAAADP HECYAKVFDE
FKPLVEEPQN LIKQNCELFE QLGEYKFQNA LLVRYTKKVP
QVSTPTLVEV SRNLGKVGSK CCKHPEAKRM PCAEDYLSVV
LNQLCVLHEK TPVSDRVTKC CTESLVNRRP CFSALEVDET
YVPKEFNAET FTFHADICTL SEKERQIKKQ TALVELVKHK
PKATKEQLKA VMDDFAAFVE KCCKADDKET CFAEEGKKLV
AASQAALGL

There were no predicted Hyp-glycosylation sites.

We expressed this in BY-2 cells and the population of molecules contained only a trace of Hyp . . . presumably because this is a folded protein and potential target Pro's (boldfaced) are not accessible to the post-translational machinery.

Add arabinogalactosylation sites by mutating L-447 and E-449 to P.

### Insulin Like Growth Factor 1 (AAA52539.1)

(SEQ ID NO: 39)

MGKISSLPTQ LFKCCFCDFL KVKMHTMSSS HLFYLALCLL
TFTSSATAGO ETLCGAELVD ALQFVCGDRG FYFNKPTGYG
SSSRRAOQTG IVDECCFRSC DLRRLEMYCA PLKPAKSARS
VRAQRHTDMP KTQKYQPOST NKNTKSQRRK GWPKTHPGGE
QKEGTEASLQ IRGKKKEQRR EIGSRNAECR GKKGK

This protein has predicted Pro-hydroxylation sites, but not predicted Hyp-glycosylation sites.

Add arabinogalactosylation sites by mutating F-42 to P, S-44 to P, and A-46 to P

### Interferon Alpha 2 (NM000605.2)

(SEQ ID NO: 40)

MALTFALLVA LLVLSCKSSC SVGCDLPQTH SLGSRRTLML
LAQMRRISLF SCLKDRHDFG FPQEEFGNQF QKAETIPVLH
EMIQQIFNLF STKDSSAAWD ETLLDKFYTE LYQQLNDLEA
CVIQGVGVTE TPLMKEDSIL AVRKYFQRIT LYLKEKKYSP
CAWEVVRAEI MRSFSLSTNL QESLRSKE

The sequence above is that of Interferon alpha2b. It differs from alpha2a at position 46 (23 of the mature sequence) (boldfaced), which is Arg in 2b and Lys in 2a.
There are no predicted Pro-hydroxylation sites in either 2a or 2b.

Introduce arabinogalactosylation sites by mutating L-176 & 184 to P, F-174 to P, T 178 to P, R-185 to S or A and K 187 to P.

### Interferon Gamma (NP00610.1)

(SEQ ID NO: 41)

MKYTSYILAF QLCIVLGSLG CYCQDPYVKE AENLKKYFNA
GHSDVADNGT LFLGILKNWK EESDRKIMQS QIVSFYFKLF
KNFKDDQSIQ KSVETIKEDM NVKFFNSNKK KRDDFEKLTN
YSVTDLNVQR KAIHELIQVM AELS#AAKTG KRKRSQMLFQ
GRRASQ

There is only one predicted Hyp-glycosylation site.
Add arabinogalactosylation by mutating Gln 166 to Pro, Arg 163 to Ser, Ala 164 to Pro

### Interferon Omega (NP002168.1)

(SEQ ID NO: 42)

MALLFPLLAA LVMTSYS#VG SLGCDLPQNH GLLSPNTLVL
LHQMRRISOF LCLKDRRDFR FPQEMVKGSQ LQKAHVMSVL
HEMLQQIFSL FHTERSSAAW NMTLLDQLHT GLHQQLQHLE
TCLLQVVGEG ESAGAISS#A LTLRRYFQGI RVYLKEKKYS
DCAWEVVRME IMKSLFLSTN MQERLRSKDR DLGSS

If the entire sequence is inputted, Pro-18 is predicted to become arabinogalactosylated-Hyp. Several signal peptide residues are within the entropy window used in predicting whether Pro-Hydroxylation occurs. Several signal peptide residues are also within the 11-aa window used for prediction of Hyp-glycosylation. If only the mature sequence is input, Pro-18 is not predicted to be hydroxylated.

Hence, there is only one predicted Hyp-glycosylation site Pro-139). However, if the mature sequence is inputted into the secondary structure prediction program HNN, it is found that this Pro-139 lies at the second position of a predicted alpha-helix.

There are also cysteines in this protein.

Introduce arabinogalactosylation sites by mutating G-20 to P and L-22 to P.

### Interleukin 10 (NP000563.1)

(SEQ ID NO: 45)

MHSSALLCCL VLLTGVRASO GQGTQSENSC THFPGNLPNM
LRDLRDAFSR VKTFFQMKDQ LDNLLLKESL LEDFKGYLGC
QALSEMIQFY LEEVMPQAEN QDPDIKAHVN SLGENLKTLR
LRLRRCHRFL PCENKSKAVE QVKNAFNKLQ EKGIYKAMSE
FDIFINYIEA YMTMKIRN

This protein has predicted Pro-hydroxylation sites, but not predicted Hyp-glycosylation sites.
Add glycosylation by mutating Gln 22 to Pro and Thr 24 to Pro

### Insulin-Like Growth Factor I (AAA52539.1)

(SEQ ID NO: 47)

MGKISSLPTQ LFKCCFCDFL KVKMHTMSSS HLFYLALCLL
TFTSSATAGO ETLCGAELVD ALQFVCGDRG FYFNKPTGYG
SSSRPAOQTG IVDECCFRSC DLRRLEMYCA PLKPAKSARS
VRAQRHTDMP KTQKYQPOST NKNTKSQRRK GWPKTHPGGE
QKEGTEASLQ IRGKKKEQRR EIGSRNAECR GKKGK

This protein has predicted Pro-hydroxylation sites, but not predicted Hyp-glycosylation sites.

Add arabinogalactosylation sites by mutating S-29 and H-31 to P

### Monocyte Chemotactic Protein-1 (NP002973.1)

(SEQ ID NO: 49)

MKVSAALLCL LLIAATFIPQ GLAQPDAINA PVTCCYNFTN
RKISVQRLAS YRRITSSKCP KEAVIFKTIV AKEICADPKQ
KWVQDSMDHL DKQTQTPKT

To introduce arabinogalactosylation sites, alter the extreme C-terminal Q's to S or A.

### Table P: Non-Plant Proteins Previously Expressed in Plants

The plant expressed proteins are described in the following format: Protein name (host plant cell species, promoter, signal peptide, yield, references). The signal peptide in the protein sequence is italicized. Pro residues in protein sequence are bold (this doesn't mean that they are hydroxylated or glycosylated). N-glycosylation sites are “redlined”.

For each protein, we have determined whether our most preferred preliminary prediction method (the standard quantitative method, with the revised matrix, for predicting Pro-Hydroxylation, and the new standard method for predicting Hyp-glycosylation of the predicted Pro-Hydroxylation (Hyp) sites) predicts any such sites, and we indicate the locations of predicted plain Hyp, Ara-Hyp, and AraGal-Hyp.

Green Fluorescent Protein, GFP (Tobacco cell suspension culture, CaMV 35S promoter, Arabidopsis basic chitinase signal peptide, 50% secreted, 12 mg/L; Su et al., High-level secretion of functional green fluorescent protein from transgenic tobacco cell cultures: characterization and sensing. Biotechnol. Bioeng. 85, 610-619, 2004).

```
(SEQ ID NO: 70)
    1 mvskgeelft gvv [symbol] ilveld gdvnghkfsv sgegegdaty gkltlkfict tgklpvpwpt
  61 lvttltygvq cfsrypdhmk qhdffksamp egyvqertif fkddgnyktr aevkfegdtl
121 vnrielkgid fkedgnilgh kleynynshn vyimadkqkn gikvnfkirh niedgsvqla
181 dhyqqntpig dgpvllpdnh ylstqsalsk dpnekrdhmv llefvtaagi tlgmdelyk
```

See the Examples for the related enhanced Green Fluorescent Protein (SEQ ID NO:35), which has no predicted Pro-Hydroxylation sites.
Human serum albumin (Tobacco cell suspension culture, CaMV 35S promoter, tobacco extensin signal peptide, secreted, 5-10 mg/L detected in this lab; Tobacco leaves Chloroplasts, 11% TSP, Plant Biotechnol. J. 1, 71-79, 2003; Potato and tobacco plant, CaMV35S promoter, tobacco PR-S signal peptide, 0.02% TSP, Sijmons et al., Bio/Technology, 8:217-221, 1990)
Signal sequence not shown here

```
(SEQ ID NO: 71)
    1 dahksevahr fkdlgeenfk alvilafaqy lqqcpfedhv klvnevtefa ktcvadesae
  61 ncdkslhtlf gdklctvatl retygemadc cakqeperne cflqhkddnp nlprlvrpev
121 dvmctafhdn eetflkkyly eiarrhpyfy apellffakr ykaafteccq aadkaacllp
181 kldelrdegk assakqrlkc aslqkfgera fkawavarls qrfpkaefae vsklvtdltk
241 vhtecchgdl lecaddradl akyicenqas issklkecce kpllekshci aevendempa
301 dlpslaadfv eskdvcknya eakdvflgmf lyeyarrhpd ysvvlllrla ktyettlekc
361 caaadphecy akvfdefkpl veepqnlikq ncelfeqlge ykfqnallvr ytkkvpqvst
421 ptlvevsrnl gkvgskcckh peakrmpcae dylsvvlnql cvlhektpvs drvtkcctes
481 lvnrrpcfsa levdetyvpk efnaetftfh adictlseke rqikkqtalv elvkhkpkat
541 keqlkavmdd faafvekcck addketcfae egkklvaasq aalgl
```

See the Examples (SEQ ID NO:37); there were no predicted Pro-hydroxylation sites.
Human a₁-antitrypsin (Rice cell suspension culture, RAmy3D promoter, RAmy3D signal peptide, secreted, 85 mg/L in shake flask, 25 mg/L in bioreactor; Terashima, M. et al. Production of functional human a₁-antitrypsin by plant cell culture. Appl. Microbiol. Biotechnol. 52, 516-523, 1999)

```
(SEQ ID NO: 72)
    1 mpssvswgil laglcclvpv slaedpqgda aqktdtshhd qdhptfnkit pnlaefafsl
  61 yrqlahqsns tniffspvsi atafamlslg tkadthdeil eglnf [symbol] tei peaqihegfq
121 ellrtlnqpd sqlqlttgng lflseglklv dkfledvkkl yhseaftvnf gdheeakkqi
181 ndyvekgtqg kivdlvkeld rdtvfalvny iffkgkwerp fevkdteded fhvdqvttvk
241 vpmmkrlgmf niqhckklss wvllmkylg [symbol] ataifflpde gklqhlenel thdiitkfle
301 nedrrsaslh lpklsitgty dlksvlgqlg itkvfsngad lsgvteeapl klskavhkav
361 ltidekgtea agamfleaip msippevkfn kpfvflmieq ntksplfmgk vvnptqk
```

No predicted Pro-hydroxylation sites.
Bryodin 1 (BD1) (Tobacco cell suspension culture, CaMV 35S promoter, tobacco extensin signal peptide, secreted, 30 mg/L; Francisco, J. A. et al. Expression and characterization of bryodin 1 and a bryodin 1-based single chain immunotoxin from tobacco cell culture. Bioconjug. Chem. 8, 708-713, 1997)

```
(SEQ ID NO: 73)
    1 mikilvlwll iltiflkspt vegdvsfrls gatttsygvf iknlrealpy erkvynipll
  61 rssisgsgry tllhltnyad etisvavdvt nvyimgylag dvsyffneas ateaakfvfk
121 dakkkvtlpy sgnyerlqta agkirenipl glpaldsait tlyyytassa asallvliqs
181 taesarykfi eqqigkrvdk tflpslatis len [symbol] wsalsk qiqiastnng qfespvvlid
241 gnnqrvsit [symbol]  asarvvtsni alllnrnnia
```

No predicted Pro-hydroxylation sites.
Hepatitis B surface antigen (HBsAg) (Retained intracellular up to 22 mg/L in soybean and 2 mg/L in tobacco, (ocs)mas promoter, native signal peptide, Smith, M. L. et al. Hepatitis B surface antigen (HbsAg) expression in plant cell culture: kinetics of antigen accumulation in batch culture and its intracellular form. Biotechnol Bioeng. 80(7):812-822, 2002; Tobacco BY-2 cells, CaMV35S promoter, soybean gene vspA signal peptide, 226 ng/mg TSP, Sojikul et al., PNAS, 100(5):2209-2214; Potato tubers and leaves, CaMV35S promoter with dual enhancer, soybean VSP “aS” signal peptide or native signal peptide, <0.05% TSP, Richter et al., Nat. Biotechnol. 18:1167-1171, 2000)

```
(SEQ ID NO: 74)
    1 mesttsgflg [symbol] llvlqagff lltriltipq sldswwtsln flggaptcpg qnsqspts [symbol] h
  61 sptscpptcp gyrwmclrrf iiflfilllc lifllvlldy qgmlpvcpll pgtsttstgp
121 crtctipaqg tsmfpsccct kpsdg [symbol] ctci pipsswafar flwewasvrf swlsllvpfv
181 qwfvglsptv wlsaiwmmwy wgpslynils pflpllpiff clwvyi
```

AraGal-Hyp predicted at Pro-56, Pro-62; Hyp at Pro-288.
mAb against HBsAg (Tobacco BY-2 cell suspension culture, CaMV 35S promoter, signal peptide of calreticulin of Nicotiana plumbaginfolia or signal peptide of hordothionin of barley. secreted, 2-7.5 mg/L; Yano, A. et al. Transgenic tobacco cells producing the human monoclonal antibody to Hepatitis B virus surface antigen. J. Med. Virol. 73, 208-215, 2004)

### Heavy Chain

```
(SEQ ID NO: 75)
    1 melglswvlf aallrgvqcq eqlvesgggv vqpgkslrls caasgftfss fpmqwvrqap
  61 gkglewvali wydgsykyya davkgrftis rdnskntvyv qlnslraedt avyycargfy
121 eaymdvwgkg ttvtvss
```

No predicted Pro-hydroxylation sites.

### Light Chain

```
(SEQ ID NO: 76)
    1 mdmgapaqll fllllwlpda tgeivltqsp gtlslspger atfscrasqs vsgsylawyq
  61 qkpgqaprll iygassratg vpdrfsgsgs gtdftltisr lqpadfavyy cqqygsfpyt
121 fgpgtkvdik r
```

No predicted Pro-hydroxylation sites.
Human Interleukin-12 (N. tabacum cv Havana suspension culture, Enhanced CaMV 35S promoter, native signal peptide, secreted, 800 ug/L; Kwon, T. H. et al. Expression and secretion of the heterodimeric protein interleukin-12 in plant cell suspension culture. Biotechnol Bioeng 81(7):870-875, 2002)

```
35 kDa subunit
(SEQ ID NO: 77)
    1 mw [symbol] [symbol] gsasq [symbol] [symbol] [symbol]   spaaatgl h [symbol] aar [symbol] vslq crlsmc [symbol] ars lllvatlvll dhlslarnlp
  61 vatpdpgmfp clhhsqnllr avsnmlqkar qtlefypcts eeidheditk dktstveacl
121 pleltk [symbol] esc lnsretsfit [symbol] gsclasrkt sfmmalclss iyedlkmyqv efktmnakll
181 mdpkrqifld qnmlavidel mqalnfnset vpqkssleep dfyktkiklc illhafrira
241 vtidrvmsyl [symbol] as
```

Ara-Hyp (@) predicted at Pro-64.

```
40 kDa subunit
(SEQ ID NO: 78)
    1 mchqqlvisw fslvflas [symbol] l vaiwelkkdv yvveldwypd apgemvvltc dtpeedgitw
  61 tldqssevlg sgktltiqvk efgdagqyte hkggevlshs llllhkkedg iwstdilkdq
121 kepk [symbol] ktflr ceak [symbol] ysgrf tcwwlttist dltfsvkssr gssdpqgvtc gaatlsaerv
181 rgdnkeyeys vecqedsacp aaeeslpiev mvdavhklky e [symbol] ytssffir diikpdppkn
241 lqlkplknsr qvevsweypd twstphsyfs ltfcvqvqgk skrekkdrvf tdktsatvic
301 rk [symbol] asisvra qdryysssws ewasvpcs
```

No predicted Pro-hydroxylation sites.
Single chain Fv antibody against HBsAg (N. tabacum cell suspension culture, CaMV 35S promoter, sporamin signal peptide, secreted, 1.0 mg/L; Ramirez, N. et al. Single-chain antibody fragments specific to the hepatitis B surface antigen, produced in recombinant tobacco cell cultures, Biotechnol Lett. 22: 1233-1236, 2000)

```
(SEQ ID NO: 79)
    1 maevqlvesg gglvkpggsl rlscadsgft fsdyymswir qapgkglewv syisssgsti
  61 yyadsvkgrf tisrdnakns lylqmnslra edtavyycar klrngrwplv ywgqgtlvtv
121 srggggsggg gsggggssel tqdpavsval gqtvritcqg dslrsyyasw ygqkpgqapv
181 lviygknnrp sgipdrfsgs ssgntaslti tgaqaedead yycnsrdssg nhvvfgggtk
241 ltvlgaaaeq kilseeding aa
```

No predicted Pro-hydroxylation sites.
Carrot Invertase (Tobacco cell suspension culture, CaMV35S promoter, native signal sequence, 1.6 mg/L in cells; Des Molles et al., J. Biosci Bioeng., 87, 302-306, 1999)

```
(SEQ ID NO: 80)
    1 mnttciavsn mrpccrmlls cknssifgys frkcdhrmgt [symbol] lskkqfkvy glrgyvscrg
  61 gkgigyrcgi dpnrkgffgs gsdwgqprvl tsgcrrvdsg grsvlvnvas dyr [symbol] hstsve
121 ghvndksfer iyvrgglnvk plviervekg ekvreeegrv gv [symbol] gsnvnig dskglnggkv
182 lspkrevsev ekeawellrg avvdycgnpv gtvaasdpad stplnydqvf irdfvpsala
241 fllngegeiv knfllhtlql qswektvdch spgqglmpas fkvknvaidg kigesedild
301 pdfgesaigr vapvdsglww iillraytkl tgdyglqarv dvqtgirlil nlcltdgfdm
361 fptllvtdgs cmidrrmgih ghpleiqalf ysalrcsrem liv [symbol] dstknl vaavnnrlsa
421 lsfhireyyw vdmkkineiy rykteeystd ainkfniypd qipswlvdwm petggylign
481 lqpahmdfrf ftlgnlwsiv sslgtpkq [symbol] e silnliedkw ddlvahmplk icypaleyee
541 wrvitgsdpk ntpwsyhngg swptllwqft lacikmkkpe larkavalae kklsedhwpe
601 yydtrrgrfi gkqsrlyqtw tiagfltskl llenpemask lfweedyell escvcaigks
661 grkkcsrfaa ksqvv
```

No predicted Pro-hydroxylation sites.
Human erythropoietin (Tobacco BY-2 cell suspension culture, CaMV 35S promoter, native signal peptide, secreted, 1 pg/gFW; Matsumoto, S. et al. Characterization of a human glycoprotein (erythropoietin) produced in cultured tobacco cells. Plant Mol. Biol. 27, 1163-1173, 1995)

```
(SEQ ID NO: 81)
    1 mgvhecpawl wlllsllslp lglpvlgapp rlicdsrvle rylleakeae [symbol] ittgcaehc
  61 slne [symbol] itvpd tkvnfyawkr mevgqqavev wqglallsea vlrgqallv [symbol] ssqpweplql
121 hvdkavsglr slttllralg aqkeaisppd aasaaplrti tadtfrklfr vysnflrgkl
181 klytgeacrt gdr
```

See the Examples at SEQ ID NO:22, one predicted Ara-Hyp; one predicted Hyp.
Human lactoferrin (Tobacco BY-2 cell suspension culture, Oxidative stress-inducible peroxidase (SWPA2) promoter, tobacco ER calreticulin signal peptide, 4.3W TSP; Choi, S. M. et al. High expression of a human lactoferrin in transgenic tobacco cell cultures. Biotechnol. Lett. 25: 213-218, 2003)

```
(SEQ ID NO: 82)
    1 mklvflvllf lgalglclag rrrrsvqwct vsqpeatkcf qwqrnmrrvr gppvscikrd
  61 spiqciqaia enradavtld ggfiyeagla pyklrpvaae vygterqprt hyyavavvkk
121 ggsfqlnelg glkschtglr rtagwnvpig tlrpfl [symbol] wtg ppepieaava rffsascvpg
181 adkgqfpnlc rlcagtgenk cafssqepyf sysgafkclr dgagdvafir estvfedlsd
241 eaerdeyell cpdntrkpvd kfkdchlarv pshavvarsv ngkedaiwnl lrqaqekfgk
301 dkspkfqlfg spsgqkdllf kdsaigfsrv ppridsglyl gsgyftaiqn lrkseeevaa
361 rrarvvwcav geqelrkcnq wsglsegsvt cssasttedc ialvlkgead amsldggyvy
421 tagkcglvpv laenyksqqs sdpdpncvdr pvegylavav vrrsdtsltw nsvkgkksch
481 tavdrtagwn ipmgllf [symbol] qt gsckfdeyfs qscapgsdpr snlcalcigd eqgenkcvpn
541 sneryygytg afrclaenag dvafvkdvtv lqntdgnnne awakdlklad fallcldgkr
601 kpvtearsch lamapnhavv srmdkverlk qvllhqqakf gr [symbol] agsdcpdk fclfqsetkn
661 llfndntecl arlhgkttye kylgpqyvag itnlkkcsts plleaceflr k
```

Ara-Hyp predicted at Pro-304; Hyp at Pro-53, Pro-162, Pro-312, Pro-332.
Human hirudin (Arabidopsis, Arabidopsis oleosin promoter, 1% seed weight; Parmenter D. et al. Production of biologically active hirudin in plant seeds using oleosin partitioning. Plant Mol Biol. 29(6):1167-80, 1995)
Signal sequence not shown here

(SEQ ID NO: 83)
1 vvytdctesg qnlclcegsn vcgqgnkcil gsdgeknqcv tgegtpkpqs hndgdfeeip
61 eeylq

No predicted Pro-hydroxylation sites.
Human milk β-casein (Solanum tuberosum (Potato) leaves, Auxin-inducible mannopine synthase promoter, native signal sequence, 0.01% TSP, Chong et al., Transgenic Res., 6, 289-296, 1997)

```
(SEQ ID NO: 84)
    1 mkvlilaclv alalaretie slssseesit eykqkvekvk hedqqqgede hqdkiypsfq
  61 pqpliypfve pipygflpqn ilplaqpavv lpvpqpeime vpkakdtvyt kgrvmpvlks
121 ptipffdpqi pkltdlenlh lplpllqplm qqvpqpipqt lalppqplws vpqpkvlpip
181 qqvvpypqra vpvqalllnq elllnpthqi ypvtqplapv hnpisv
```

AraGal-Hyp predicted at Pro-94, Pro-172, Pro-185; Hyp at Pro-165, Pro-219.
Human milk CD14 protein (Tobacco cell culture, CaMV35S promoter, native signal sequence or tomato extensin signal peptide, 5 ug/L medium, Girard et al., Plant Cell, Tissue and Organ Culture 78: 253-260, 2004

```
(SEQ ID NO: 85)
    1 merascllll ll [symbol] lvhvsat tpepceldde dfrcvc [symbol] fse pqpdwseafq cvsaveveih
  61 agglnlepfl krvdadadpr qyadtvkalr vrrltvgaaq vpaqllvgal rvlaysrlke
121 ltledlkitg tmpplpleat glalsslrlr [symbol] vswatgrsw laelqqwlkp glkvlsiaqa
181 hspafsceqv rafpaltsld lsdnpglger glmaalcphk fpaiqnlalr ntgmetptgv
241 caalaaagvq phsldlshns lratvnpsap rcmwssalns l [symbol] lsfagleq vpkglpaklr
301 vldlscnrln rapqpdelpe vd [symbol] ltldgnp flvpgtalph egsmnsgvvp acarstlsvg
361 vsgtlvllqg argfa
```

AraGal-Hyp predicted at Pro-183, Pro-313; Ara-Hyp at Pro-22; Hyp at Pro-134.
Human granulocyte-macrophage colony-stimulating factor (hGM-CSF) (Rice cell suspension culture, Ramy3D promoter, Ramy3D signal peptide, secreted 125 mg/L; Shin et al., Biotechnol. Bioeng. 82 (7): 778-783, 2003; Tomato cell suspension culture, duplicated CaMV 35S promoter, omega mRNA signal sequence from the coat protein gene of tobacco mosaic virus, secreted 45 ug/L, Kwon et al., Biotechnol. Lett. 25 (18): 1571-1574, 2003; Tobacco cell suspension culture, CaMV 35S promoter, native signal sequence, secreted 270 ug/L, Kwon et al., Biotechnol. Bioprocess Bioeng. 8 (2): 135-141, 2003)

```
(SEQ ID NO: 86)
    1 mwlqsllllg tvacsisapa rspspstqpw ehvnaiqear rll [symbol] lsrdta aem [symbol] etvevi
  61 semfdlqept clqtrlelyk qglrgsltkl kgpltmmash ykqhcpptpe tscatqiitf
121 esfkenlkdf llvipfdcwe pvqe
```

See the Examples (SEQ ID NO:12), 3 predicted AraGal-Hyp, 1 predicted Ara-Hyp.
Human haemoglobin (Tobacco plant, CaMV35S promoter, chloroplastic transit signal peptide, 0.05% TSP in seed, Dieryck et al., NATURE 386 (6620): 29-30, 1997)

```
alpha globin
(SEQ ID NO: 87)
    1 mvlspadktn vkaawgkvga hageygaeal ermflsfptt ktyfphfdls hgsaqvkghg
  61 kkvadaltna vahvddmpna lsalsdlhah klrvdpvnfk llshcllvtl aahlpaeftp
121 avhasldkfl asvstvltsk yr
```

AraGal-Hyp predicted at Pro-120; Hyp at Pro-S.

```
beta globin
(SEQ ID NO: 88)
    1 mvhltpeeks avtalwgkvn vdevggealg rllvvypwtq rffesfgdls tpdavmgnpk
  61 vkahgkkvlg afsdglahld nlkgtfatls elhcdklhvd penfrllgnv lvcvlahhfg
121 keftppvqaa yqkvvagvan alahkyh
```

Hyp predicted at Pro-126.
Despite the foregoing preliminary predictions, neither globin is likely to be reliably Hyp-glycosylated without sequence modifications. The flanking sequences are low in Pro, esp B-globin.
Human epidermal growth factor (Tobacco plant, CaMV35S promoter or CaMV 35S long promoter, tobacco AP24 osmotin signal peptide, 0.015% TSP, Wirth et al., MOLECULAR BREEDING 13 (1): 23-35, 2004; Tobacco plant, CaMV35S promoter, native signal peptide, 0.001% TSP, Higo et al., Biosci. Biotech. Bioch. 57 (9): 1477-1481, 1993)

(SEQ ID NO: 89)
mrpsgtagaa llallaalc [symbol] asraleekkg kgvsrrlprr priaprtpqp aqprtgapar
61 araparpflf p

AraGal-Hyp predicted at Pro-58; Ara-Hyp at Pro-48; Hyp at Pro-45.
Human protein C (tobacco plant, CaMV35S promoter, native signal peptide, <0.01% TSP, Cramer et al., Ann NY Acad. Sci. 792:62-71, 1996)
Signal sequence not shown here

```
(SEQ ID NO: 90)
    1 eydlrrwekw eldldikevf vhp [symbol] yskstt dndiallhla qpatlsqtiv piclpdsgla
  61 erelnqagqe tlmtgwgyhs srekeakr [symbol] r tfvlnfikip vvphnecsev msnmvsenml
121 cagilgdrqd acegdsggpm vasfhgtwfl vglvswgegc gllhnygvyt kvsryldwih
181 ghirdkeapq kswap
```

No predicted Pro-Hydroxylation sites.
Human growth hormone (Tobacco BY-2 cell suspension culture, CaMV35S promoter, extensin signal peptide, secreted <0.007 mg/L, result from this lab; Tobacco seed, sorghum γ-kafirin gene promoter, alpha-coixin signal peptide, 0.16% TSP, Leite et al., MOLECULAR BREEDING 6 (1): 47-53, 2000; Tobacco chloroplasts, 7% TSP, Staub et al., Nature Biotechnol. 18 (3): 333-338, 2000)

```
(SEQ ID NO: 91)
    1 matgsrtsll lafgllclpw lqegsafpti plsrlfd [symbol] as lrahrlhqla fdtyqefeea
  61 yipkeqkysf lqnpqtslcf sesiptpsnr eetqqksnle llrisllliq swlepvqflr
121 svfanslvyg asdsnvydll kdleegiqtl mgrledgspr tgqifkqtys kfdtnshndd
181 allknyglly cfrkdmdkve tflrivqcrs vegscgf
```

See the Examples (SEQ ID NO:33), one predicted Hyp. We know experimentally that unmodified HGH isn't Hyp-glycosylated.
Human interferon alpha2b (Tobacco BY-2 cell suspension culture, CaMV35S promoter, extensin signal peptide, secreted <0.002 mg/L, result from this lab; Potato plant, CaMV35S promoter, native signal peptide, 560 IU/g, J. INTERFERON CYTOKINE RES. 21 (8): 595-602, 2001

```
(SEQ ID NO: 92)
    1 maltfyllva lvvlsyksffs slgcdlpqth slgnrralil laqmrrispf sclkdrhdfe
  61 fpqeefddkq fqkaqaisvl hemiqqtfnl fstkdssaal detlldefyi eldqqlndle
121 scvmqevgvi esplmyedsi lavrkyfqri tlyltekkys scawevvrae imrsfslsin
181 lqkrlkske
```

See the Examples, Human Interferon Alpha-2 (NM000605.2) (SEQ ID NO40). No predicted Pro-hydroxylation sites.
Human interferon beta (Tobacco plant, CaMV35S promoter, native signal peptide, 0.01% fresh weight, J. INTERFERON RES. 12 (6): 449-453, 1992)

```
(SEQ ID NO: 93)
    1 mtnkcllqia lllcfsttal smsynllgfl qrssncqcqk llwqlngrle yclkdrrnfd
  61 ipeeikqlqq fqkedaavti yemlqnifai frqdssstgw [symbol] etivenlla nvyhqrnhlk
121 tvleekleke dftrgkrmss lhlkryygri lhylkakeds hcawtivrve ilrnfyvinr
181 ltgylrn
```

No predicted Pro-Hydroxylation sites.
Human placental alkaline phosphatase (Tobacco root, CaMV 35S or mas2′ promoter, native signal peptide, 20 ug/g of root dry weight/day, Borisjuk et al., Nat. Biotechnol. 17, 466-469, 1999)

```
(SEQ ID NO: 94)
    1 mlg [symbol] cmllll lllglrlqls lgiilveeen pdfwnreaae algaakklqp aqtaaknlii
  61 flgdgvgvst vtaarilkgq kkdklgpeip lamdrfpyva lsktynvdkh vpdsgatata
121 ylcgvkgnfq tiglsaaarf nqc [symbol] ttrgne visvmnrakk agksvgvvtt trvqhaspag
181 tyahtvnrnw ysdadvpasa rqegcqdiat qlisnmdidv ilgggrkymf rmgtpdpeyp
241 ddysqggtrl dgknlvqewl akhqgaryvw [symbol] rtelmrasl dpsvahlmgl fepgdmkyei
301 hrdstldpsl memteaalrl lsrnprgffl fveggridhg hhesrayral tetimfddai
361 eragqltsee dtlslvtadh shvfsfggcp lrggsifgla pgkardrkay tvllygngpg
421 yvlkdgarpd vtesesgspe yrqqsavpld eethagedva vfargpqahl vhgvqeqtfi
481 ahvmafaacl epytacdlap pagttdaahp grsvvpallp llagtlllle tatap
```

AraGal-Hyp predicted at Pro-178, Pro-535; Ara-Hyp at Pro-235, Pro-450; Hyp at Pro-439, Pro-501, Pro-516.
Human Interleukin-2 (Tobacco cell culture, CaMV35S promoter, native signal peptide, secreted, 0.1 ug/L, Magnuson et al., Protein Expr. Purifi. 13 (1): 45-52, 1998)

```
(SEQ ID NO: 95)
    1 myrmqllsci alslalvtns aptssstkkt qlqlehllld lqmilnginn yknpkltrml
  61 tfkfympkka telkhlqcle eelkpleevl nlaqsknfhl rprdlisnin vivlelkgse
121 ttfmceyade tativeflnr witfcqsiis tlt
```

See the Examples (SEQ ID NO:25), one predicted AraGal-Hyp.
Human Interleukin-4 (Tobacco cell culture, CaMV35S promoter, native signal peptide, secreted, 0.18 ug/L, Magnuson et al., Protein Expr. Purifi. 13 (1): 45-52, 1998)

```
(SEQ ID NO: 96)
    1 mgltsqll [symbol] [symbol]  lffllacagn fvhghkcdit lqeiiktlns lteqktlcte ltvtdifaas
  61 k [symbol] tteketfc raatvlrqfy shhekdtrcl gataqqfhrh kqlirflkrl drnlwglagl
121 nscpvkea [symbol] q stlenflerl ktimrekysk css
```

No predicted Pro-Hydroxylation sites.
Human muscarinic cholinergic receptors (Tobacco plant and BY-2 cell culture, CaMV35S promoter, native signal peptide, 240 fmol/mg membrane protein. Mu et al., Plant Mol. Bio. 34 (2): 357-362, 1997)

```
m1
(SEQ ID NO: 97)
    mntsa [symbol] [symbol]  avs [symbol] nitvla [symbol] gk g [symbol] wgvafigi ttgllslatv tgnhllvlisf kvntelktvn
  61 nyfllslaca dliigtfsmn lyttyllmgh walgtlacdl wlaldyvas [symbol] asvmnlllis
121 fdryfsvtrp lsyrakrtpr raalmiglaw lvsfvlwapa ilfwqylvge rtvlagqcyi
181 qflsqpiitf gtamaafylp vtvmctlywr iyretenrar elaalqgset pgkgggssss
241 sersqpgaeg spetppgrcc rccraprllq ayswkeeeee degsmeslts segeepgsev
301 vikmpmvdpe aqaptkqppr sspntvkrpt kkgrdragkg qkprgkeqla krktfslvke
361 kkaartlsai llafiltwtp ynimvlvstf ckdcvpetlw elgywicyv [symbol] stinpmcyal
421 cnkafrdtfr llllcrwdkr rwrkipkrpg svhr
```

Hyp predicted at Pro-231, Pro-252, Pro-254, Pro-323.

```
m2
(SEQ ID NO: 98)
    1 mnnstnssnn slalts [symbol] ykt fevvfivlva gslslvtiig nilvmvsikv nrhlqtvnny
  61 flfslacadl iigvfsmnly tlytvigywp lgpvvcdlwl aldyvvs [symbol] as vmnlliisfd
121 ryfcvtkplt ypvkrttkma gmmiaaawvl sfilwapail fwqfivgvrt vedgecyiqf
181 fsnaavtfgt aiaafylpvi imtvlywhis rasksrikkd kkepvanqdp vspslvqgri
241 vkpnnnnmps sddglehnki qngkaprdpv tencvqgeek ess [symbol] dstsvs avasnmrdde
301 itqdentvst slghskdens kqtcirigtk tpksdsctpt [symbol] ttvevvgss gqngdekqni
361 varkivkmtk qpakkkppps rekkvtrtil aillafiitw apynvmvlin tfcapcipnt
421 vwtigywlcy i [symbol] stinpacy alc [symbol] atfkkt fkhllm
```

Ara-Hyp predicted at Pro-332, Pro-378; Hyp at Pro-233, Pro-379.
Human insulin-like growth factor (Tobacco plant, Maize ubiquitin promoter, Lam B signal peptide, 43 ng/mg TSP, Panahi et al., Molecular Breeding, 12:21-31, 2003)

```
(SEQ ID NO: 99)
    1 mgkissl [symbol] tq lfkccfcdfl kvkmhtmsss hlfylalcll tftssatagp etlcgaelvd
  61 alqfvcgdrg fyfnkptgyg sssrrapqtg ivdeccfrsc dlrrlemyca plkpaksars
121 vraqrhtdmp ktqkevhlkn asrgsagnkn yrm
```

See the examples, SEQ ID NO:39, no predicted glyco-Hyp, 3 predicted Hyp.
Avidin (Corn, corn ubiquitin promoter, alpha-amylase signal sequence, 2.1-5.7% TSP in seed, Kusnadi et al., Biotechnol. Prog. 14 (1): 149-155, 1998)

```
(SEQ ID NO: 100)
    1 mvhats [symbol] lll llllslalva [symbol] slsarkcsl tgkwtndlgs [symbol] mtigavnsr geftgtyita
  61 vtatsneike splhgtqnti nkrtqptfgf tvnwkfsest tvftgqcfid rngkevlktm
121 wllrssvndi gddwkatrvg iniftrlrtq ke
```

No predicted Pro-hydroxylation sites.
Human collagen alpha-1 type-I (Tobacco plant, L3 promoter, tobacco PR-S signal peptide, 50-100 ug purified collagen/100 g leaf, Merle et al., FEBS Lett. 515 (1-3): 114-118, 2002; Tobacco plant, enhanced 35S promoter, tobacco PR-S signal peptide, 10 mg/100 g plant, Ruggiero et al., FEBS Lett. 469 (1): 132-136, 2000)

```
(SEQ ID NO: 101)
      1 mfsffvdlrll lllaatallt hgqeegqyeg qdedippitc vqnglryhdr dvwkpepcri
    61 cvcdngkvlc ddvicdetkn cpgaevpege ccpvcpdgse sptdqettgv egpkgdtgpr
  121 gprgpagppg rdgipgqpgl pgppgppgpp gppglgqnfa pqlsygydek stggisvpgp
  181 mgpsgprglp gppgapgpqg fqgppgepge pgasgpmgpr gppgppgkng ddgeagkpgr
  241 pgergppgpq garglpgtag lpgmkghrgf sgldgakgda gpagpkgepg spgengapgq
  301 mgprglpger grpgapgpag argndgatga agppgptgpa gppgfpgavg akgeagpqgp
  361 rgsegpqgvr gepgppgpag aagpagnpga dgqpgakgan gapgiagapg fpgargpsgp
  421 qgpggppgpk gnsgepgapg skgdtgakge pgpvgvqgpp gpageegkrg argepgptgl
  481 pgppgerggp gsrgfpgadg vagpkgpage rgspgpagpk gspgeagrpg eaglpgakgl
  541 tgspgspgpd gktgppgpag qdgrpgppgp pgargqagvm gfpgpkgaag epgkagergv
  601 pgppgavgpa gkdgeagaqg ppgpagpage rgeqgpagsp gfqglpgpag ppgeagkpge
  661 qgvpgdlgap gpsgargerg fpgergvqgp pgpagprgan gapgndgakg dagapgapgs
  721 qgapglqgmp gergaaglpg pkgdrgdagp kgadgspgkd gvrgltgpig ppgpagapgd
  781 kgesgpsgpa gptgargapg drgepgppyp agfagppgad gqpgakgepg dagakgdagp
  841 pgpagpagppgpignvgapg akgargsagp pgatgfpgaa grvgppgpsg nagppgppgp
  901 agkeggkgpr getgpagrpg evgppgppgp agekgspgad gpagapgtpg pqgiagqrgv
  961 vglpgqrger gfpglpgpsg epgkqgpsga sgergppgpm gppglagppg esgregapga
1021 egspgrdgsp gakgdrgetg pagppgapga pgapgpvgpa gksgdrget
```

Merle paper reported hydroxyproline content of 0.68%, implying the formation of about 7 Hyp (% Hyp increased up to 9.41% if collagen co-expressed in plant cell together with Caenorhabiditis elegans/beta human chimeric proline-4-hydroxylase.)
See the Examples, SEQ ID NO:8, many predicted glyco-Hyp sites.
Phytase (Tobacco plant, CaMV35S promoter, native signal peptide, 14.4% TSP, VERWOERD et al., PLANT PHYSIOLOGY 109 (4): 1199-1205, 1995)

```
(SEQ ID NO: 102)
    1 mgvsavllpl yllsgvtsgl avpasrnqst cdtvdqgyqc fsetshlwgq yapffslane
  61 saispdvpag ckvtfaqyls rhgaryptds kgkkysalie eiqqnattfa gkyaflktyn
121 yslgaddltp fgeqelvnsg ikfyqryesl trniipfirs sgssrviasg kkfiegfqst
181 klkdpraqps qsspkidvvi seasssnntl dpgtcavfed seladtvean ftatfvpsir
241 qrlgndlsgv sltdtevtyl mdmcsfdtis tstvdtklsp fcdlfthdew inydylqslk
301 kyyghgagnp lgptqgvgya neliarlths pvhddtssnh tldsspatfp lnstlyadfs
361 hdngiisilf alglyngtkp lstttvqnit qtdgfssawt vpfasrlyve mmqcqaeqep
421 lvrvlvndrv vplhgcpada lgrctrdsfv rglsfarsgg dwaecfa
```

AraGal-Hyp predicted at Pro-13, Pro-346; Ara-Hyp at Pro-194; Hyp at Pro-331.
Xylanase (Tobacco plant, CaMV35S promoter, native signal peptide, 4.1% TSP leaves, Herbers et al., Bio/Technolo. 13 (1): 63-66, 1995)

```
(SEQ ID NO: 103)
    1 mkrkvkkmaa matsiimaim iilhsi [symbol] vla griiyd [symbol] etg thggydyelw kdygntimel
  61 ndggtfscqw snignalfrk grkfnsdkty qelgdivvey gcdynpngns ylcvygwtrn
121 plveyyives wgswrppgat pkgtitqwma gtyeiyettr vnqpsidgta tfqqywsvrt
181 skrtsgtisv tehfkqwerm gmrmgkmyev altvegyqss gyanvyknei riga [symbol] ptpap
241 sqspirrdaf siieaeey [symbol] s t [symbol] sstlqvig tpnngrgigy iengntvtys nidfgsgatg
301 fsatvatev [symbol] tsiqirsdsp tgtllgtlyv sstgswntyq tvst [symbol] iskit gvhdivlvfs
361 gpvnvdnfif srsspvpapg dntrdaysii qaedydssyg pnlqifslpg ggsaigyien
421 gysttyknid fgdgatsvta rvatq [symbol] atti qvrlgspsgt llgtiyvgst gsfdtyrdvs
481 atisntagvk divlvfsgpv nvdwfvfsks gt
```

AraGal-Hyp predicted at Pro-240, Pro-375, Pro-377; Ara-Hyp at Pro-238; Hyp at Pro-457.
beta-glucuronidase (Tobacco cell culture, CaMV35S promoter, native signal peptide, 12 IU/ml, Lee et al., J. MICROBIOL. BIOTECHNOL. 16 (5): 673-677, 2006)

```
(SEQ ID NO: 104)
    1 mslkwsacwv algqllcsca lalkggmlfp kespsrelka ldglwhfrad lsnnrlqgfe
  61 qqwyrqplre sgpvldmpvp ssfnditqea alrdfigwvw yereailprr wtqdtdmrvv
121 lrinsahyya vvwvngihvv ehegghlpfe adisklvqsg plttcrtia i [symbol] mtltphtl
181 ppgtivyktd tsmypkgyfv qdtsfdffny aglhrsvvly ttpttyiddi tvitnveqdi
241 glvtywisvq gsehfqlevq lldedgkvva hgtgnqgqlq vpsanlwwpy lmlaehpaymy
301 slevkvttte svtdyytlpv girtvavtks kflingkpfy fqgvnkheds dirgkgfdwp
361 llvkdfnllr wlgansfrts hypyseevlq lcdrygivvi decpgvgivl pqsfg [symbol] eslr
421 hhlevmeelv rrdknhpavv mwsvanepss alkpaayyfk tlithtkald ltrpvtfvsn
481 akydadlgap yvdvicvnsy fswyhdyghl eviqpqlnsq fenwykthqk piiqseygad
541 aipgihedpp rmfseeyqka vlenyhsvld qkrkeyvvge liwnfadfmt [symbol] qsplrvign
601 kkgiftrqrq pktsafilre rywria [symbol] etg ghgsgprtqc fgsrpftf
```

AraGal-Hyp predicted at Pro-223; Hyp at Pro-182.
Aprotinin (Maize seeds, maize ubiquitin promoter, barley alpha-amylase signal peptide, 0.07% TSP, Zhong et al., MOLECULAR BREEDING 5 (4): 345-356, 1999)

(SEQ ID NO: 102)
1 rrpdfclepp ytgpckarii ryfynakagl cqtfvyggcr akrnnfksae dcmrtcgga

No predicted Hyp-glycosylation sites.
Heat-labile enterotoxin B subunit (Potato plant, CaMV35S promoter, native signal peptide, 0.01% TSP, Mason et al., vaccine 16(3):1336-1343, 1996)

```
(SEQ ID NO: 106)
    1 mnkvkcyvlf tallsslyah gapqtitelc seyrntqiyt indkilsyte smagkremvi
  61 itfksgetfq vevpgsqhid sqkkaiermk dtlritylte tkidklcvwn [symbol] ktpnsiaai
121 smkn
```

No predicted Hyp-glycosylation sites.
Norwalk virus capsid protein (Tobacco leaves and potato tubers, CaMV35S promoter or patatin promoter, native signal peptide, 0.23% TSP, Mason et al., PNAS, 93 (11): 5335-5340, 1996)

```
(SEQ ID NO: 107)
    1 mkmasndatp sndgaaglvp einneamald pvagaaiaap ltgqqniidp wimnnfvqap
  61 ggeftvsprn spgevllnle lgpeinpyla hlarmyngya ggfevqvvla gnaftagkii
121 faaippnfpi d [symbol] lsaaqitm cphvivdvrq lepvnlpmpd vrnnffhynq gsdsrlrlia
181 mlytplra [symbol] n sgddvftvsc rvltrpspdf sfnflvpptv esktkpftlp iltisemsns
241 rfpvpidslh tsptenivvq cqngrvtldg elmgttqllp sqicafrgvl trstsrasdq
301 adtatprlfn yywhiqldnl [symbol] gtpydpaed ipgplgtpdf rgkvfgvasq rnpdsttrah
361 eakvdttagr ftpklgslei stesgdfdqn qptrftpvgi gvdneadfqq wslpdysgqf
421 thnmnlapav apnfpgeqll ffrsqlpssg grsngildcl vpqewvqhfy qesapagtqv
481 alvryvnpdt grvlfeaklh klgfmtiakn gdspitvppn gyfrfeswvn pfytlapmgt
541 gngrrriq
```

### AraGal-Hyp at Pro-208, Pro-253, Pro-475; Ara-Hyp at Pro-217; Hyp at Pro-40, Pro-72, Pro-218, Pro-428.

Chymosin (Tobacco and potato plant, CaMV35S promoter, native signal peptide, 0.1-0.5% TSP, Willmitzer at al., international patent WO 92/01042)

```
(SEQ ID NO: 108)
    1 mrclvvllav falsqgteit riplykgksl rkalkehgll edflqkqqyg isskysgfge
  61 vasvpltnyl dsqyfgkiyl gtppqeftvl fdtgssdfwv psiycksngc knhqrfdprk
121 sstfqnlgkp lsihygtgsm qgilgydtvt vsnivdiqqt vglstqepgd vftyaefdgi
181 lgmaypslas eysipvfdnm mnrhlvaqdl fsvymdrngq esmltlgaid psyytgslhw
241 vpvtvqqywq ftvdsvtisg vvvaceggcq aildtgtskl vgpssdilni qqaigatqnq
301 ygefdidcd [symbol] lsymptvvfe ingkmypltp saytsqdqgf ctsgfqse [symbol] h sqkwilgdvf
361 ireyysvfdr annlvglaka i
```

Hyp predicted at Pro-83.
Cholera toxin B subunit (Tomato plant, CaMV35S promoter, native signal peptide, 0.02%-0.04% TSP, Jani et al., Transgenic Res. 11 (5): 447-454, 2002; Tobacco plant, ubiquitin promoter, native signal peptide, 1.8% TSP, Kang et al., MOLECULAR BIOTECHNOLOGY 32 (2): 93-100, 2006

```
(SEQ ID NO: 109)
    1 miklkfgvff tvllssayah gtpqnitdlc aeyhntqiyt lndkifsyte slagkremai
  61 itfkngaifq vevpgsqhid sqkkaiermk dtlriaylte akveklcvwn [symbol] ktphaiaai
121 sman
```

No predicted Pro-hydroxylation sites.
Rabies virus glycoprotein (Tomato, CaMV35S promoter, native signal peptide, 0.1% TSP,

### McGarvey et al., Nature Bio/Technol. 13 (13): 1484-1487 DEC 1995

```
(SEQ ID NO: 110)
    1 mdadkivfkv nnqvvslkpe iivdqyeyky paikdlkkps itlgkapdls kayksilsgm
  61 naakldpddv csylaaamqf fegscpddwt sygiliarrg dkitpaslvd ikrtdvegnw
121 altggmeltr dptvsehasl vglllslyrl skisgqntgn yktniadrie qifetapfak
181 ivehhtlmtt hkmca [symbol] wsti pnfrflagty dmffsriehl ysairvgtvv tayedcsglv
241 sftgfikqi [symbol] ltareallyf fhknfeeeir rmfepgqeta vphsyfihfr slglsgkspy
301 ssnavghvfn lihfvgcymg qvrsl [symbol] atvi atcaphemsv lggylgeeff gkgtferrff
361 rdekelqeye aaeltraeta laddgtvnsd dedyfssetr speavytrim mnggrlkrsh
421 irryvsvssn hqtrpnsfae fl [symbol] ktyssds
```

Hyp predicted at Pro-105, Pro-299.
Foot and mouth disease virus VP1 protein (Alfalfa plant, CaMV35S promoter, no signal peptide, yield not shown, Wigdorovitz et al., VIROLOGY 255 (2): 347-353, 1999)
Signal sequence not shown here

```
(SEQ ID NO: 111)
    1 ttstgesadp vtatvenygg etqvqrrhht dvsfildrfv kvtpkdqinv ldlmqtppht
  61 lvgallrtat yyfadlevav khegdltwvp ngapeaal [symbol] [symbol] tt [symbol] ptayhka pltrlalpyt
121 aphrvlatvy ngnckyaegs ltnvrgdlqv laqkaarplp tsfnygaika trvtellyrm
181 kraetycprp llavhpdgar hnqelvapvk qsl
```

Hyp predicted at Pro-94, Pro-111, Pro-208.
Gastroenteritis coronavirus glycoprotein S (Arabidopsis plant, CaMV35S promoter, native signal peptide, 0.006-0.03% TSP, Gomez et al., VIROLOGY 249 (2): 352-358, 1998)

```
(SEQ ID NO: 112)
      1 mkklfvvlvv m [symbol] liygdnfp csklt [symbol] rtig nqwnhietfl l [symbol] yssrlppn sdvvlgdyfp
    61 tvqpwfncir [symbol] nsndlyvtl enlkalywdy ate [symbol] itwnhr qrlnvvvngy pysitvtttr
  121 nfnsaegaii cickgspptt ttessltcnw gsecrlnhkf picpsnsean cgnmlyglqw
  181 fadevvaylh gasyrisfen qwsgtvtfgd mrattlevag tlvdlwwfnp vydvsyyrvn
  241 nk [symbol] gttvvs [symbol] ctdgcasyva nvfttqpggf ipsdfsfnnw fllt [symbol] sstlv sgklvtkqpl
  301 lvnclwpvps feeaastfcf egagfdqcng avl [symbol] ntvdvi rfnl [symbol] fttnv qsgkgatvfs
  361 l [symbol] ttggvtle iscytvsdss ffsygeipfg vtdgprycyv hy [symbol] gtalkyl gtlppsvkei
  421 aiskwghfyi ngynffstfp idcisf [symbol] ltt gdsdvfwtia ytsytealvq ventaitkvt
  481 ycnshvnnik csqitanlnn gfypvsssev glv [symbol] ksvvll psfythtiv [symbol] itiglgmkrs
  541 gygqpiastl s [symbol] itlpmqdh ntdvycirsd qfsvyvhstc ksalwdnifk r [symbol] ctdvldat
  601 aviktgtcpf sfdklnnylt fnkfclslsp vganckfdva artrtneqvv rslyviyeeg
  661 dnivgvpsdn sgvhdlsvlh ldsctdyniy grtgvgiirq t [symbol] rtllsgly ytslsgdllg
  721 fk [symbol] vsdgviy svtpcdvsaq aavidgtivg aitsinsell glthwtttpn fyyysiy [symbol] yt
  781 ndrtrgtaid sndvdcepvi tysnigvckn gafvfi [symbol] vth sdgdvqpist g [symbol] vtipt [symbol] ft
  841 isvqveyiqv yttpvsidcs ryvcngnprc nklltqyvsa cqtieqalam garlenmevd
  901 smlfvsenal klasveaf [symbol] s setldpiyke wpniggswle glkyilpshn skrkyrsaie
  961 dllfdkvvts glgtvdedyk rctggydiad lvcaqyyngi mvlpgvanad kmtmytasla
1021 ggitlgalgg gavaipfava vqarlnyval qtdvlnknqq ilasafnqai g [symbol] itqsfgkv
1081 ndaihqtsrg latvakalak vqdvvniqgq alshltvqlq nnfqaisssi sdiynrldel
1141 sadaqvdrli tgrltalnaf vsqtltrqae vrasrqlakd kvnecvrsqs qrfgfcg [symbol] gt
1201 hlfslanaap ngmiffhtvl lptayetvta wpgicasdgd rtfglvvkdv qltlfrnldd
1261 kfyltprtmy qprvatssdf vqiegcdvlf v [symbol] atvsdlps iipdyidi [symbol] q tvqdilenfr
1321 p [symbol] wtvpeltf dif [symbol] atyl [symbol] l tgeiddlefr seklh [symbol] ttve lailidni [symbol] n tlvnlewlnr
1381 ietyvkwpwy vwlliglvvi fciplllfcc cstgccgcig clgscchsic srrqfenyep
1441 iekvhvh
```

Ara-Hyp predicted at Pro-137; Hyp at Pro-138, Pro-415, Pro-854.
Avian reovirus sigma C protein (Alfalfa plant, CaMV 35S promoter and rice actim promoter, native signal peptide, 0.007-0.008% TSP, Huang et al. J. VIROLOGICAL METHODS 134 (1-2): 217-222, 2006)

```
(SEQ ID NO: 113)
    1 maglnpsqrr evvslilslt snvnishgdl tpiyerltnl eastellhrs isdisttvs [symbol]
  61 isanlqdmth tlddvtanld glrttvtalq dsvsilst [symbol] v tdlt [symbol] rssah aailsslqtt
121 vdg [symbol] staisn lksdissngl aitdlqdrvk slestashgl sfspplsvad gvvsldmdpy
181 fcsqrvslts ysaeaqlmqf rwmargt [symbol] gs sdtidmtvna hchgrrtdym msstg [symbol] ltvt
241 snvvlltfdl sdithipsdl arlvpsagfq aasfpvdvsf trdsathayq aygvysssrv
302 ftitfptggd gtanirsltv rtgidt
```

Ara-Hyp predicted at Pro-164; Hyp at Pro-165.
Despite the foregoing preliminary prediction, reliable Hyp-glycosylation is doubtful because Avian reovirus sigma C1 has a SPP sandwiched between Cys residues and the nearest flanking Pro is 14 residues away.
HIV-1 p24 antigen (Tobacco plant, CaMV35S promoter, murine immunoglobulin signal sequence, 0.1% TSP HIV-1 p24 alone, 1.4% TSP when fused to IgA., Obregon P et al., PLANT BIOTECHNOL. J. 4 (2): 195-207, 2006)
Signal sequence not shown here

```
(SEQ ID NO: 114)
    1 spevipmfsa lsegatpqdl ntmlntvggh qaamqmlket indeaaewdr lhpvqagpva
  61 pgqmreprgs diagttstlq eqinwmtgnp pipvgeiykr wiilglnkiv rmysptsild
121 ikqgpkepfr dyv
```

Hyp predicted at Pro-2.
Antibody versus Glycoprotein D of herpes simplex virus, Human IgA1 heavy chain (Maize seeds, no information on promoter and signal peptide, no information on yields. Karnoup et al., GLYCOBIOLOGY 15 (10): 965-981, 2005) Up to six proline/hydroxyproline conversions and variable amounts of arabinosylation (Pro/Hyp+Ara) were found in the hinge region (highlighted, and asterisks underneath)

```
(SEQ ID NO: 115)
    1 mefglswvfl vailkgvhce vqlvesgggl vqpggslkls caasgftlsg snvhwvrqas
  61 gkglewvgri krnaesdata yaasmrgrlt isrddsknta flqmnslksd dtamyycvir
121 gdvynrqwgq gtlvtvssas ptspkvfpls lcstqpdgnv viaclvqgff pqeplsvtws
181 esgqgvtarn fppsqdasgd lyttssqltl patqclagks vtchvkhyt [symbol] psq [symbol]
                                                                                                                          *******
241 [symbol] lslhrp aledlllgse a [symbol] ltctltgl rdasgvtftw
        ********** ********** ****
301 tpssgksavq gppdrdlcgc ysvssvlsgc aepwnhgktf tctaaypesk tpltatlsks
361 gntfrpevhl lpppseelal nelvtltcla rgfspkdvlv rwlqgsqelp rekyltwasr
421 qepsqgtttf avtsilrvaa edwkkgdtfs cmvghealpl aftqktidrl agkpthv [symbol] vs
481 vvmaevdgtc y
```

Predicted processing of hinge region is as follows:

### DVTVPCPV#ST@OT@S#ST@OT@SPSCCHPR (AAs 234-264 of SEQ ID NO:115)

Anti-rabies virus mAb (tobacco BY-2 cells, CaMV35S promoter with duplicated upstream B domains (Ca2p) and potato proteinase inhibitor II promoter (Pin2p), native signal peptide, KDEL ER retention signal, 0.5 mg/L retained in cells, Girard et al., BIOCHEMICAL AND BIOPHYSICAL RESEARCH COMMUNICATIONS 345 (2): 602-607, 2006)
Signal sequence not shown here

### Heavy Chain

```
(SEQ ID NO: 116)
    1 evqlvqsggg vvqpgrslrl scaasgftfs sysmhwvrqa pgrglewvav isydgsnkyy
  61 adsvkgrfti srdnskntly lqmnslraed tavyycvirt pqfaqyyfds wgqgtlvtvs
121 s
```

No predicted Pro-hydroxylation sites.

### Light Chain

(SEQ ID NO: 117)
1 diqltqspss vsasvgdrvt itcrasqgis swlawyqqkp gkaprsliyd asslqsgvps
61 rfsgsgsgtd ftltisslqp edfatyycqq adsfpitfgq gtrleik

AraGal-Hyp predicted at Pro-8.
Endo-1,4-beta-D-glucanase (Tobacco BY-2 suspension cells and leaves of Arabidopsis thaliana plants, CaMV35S promoter, Tobacco PR (Pathogenesis-Related)-S signal peptide, up to 26% TSP in leaves of A. thaliana. Ziegler et al., Molecular Breeding 6:37-46, 2000.
See examples at SEQ ID NO:10.
Chimeric L6 sFv anti-tumor antibody (Tobacco NT1 cells, CaMV 35S promoter, tobacco extensin signal peptide, 25 mg/L, 10% TSP, Russell and James, U.S. Pat. No. 6,080,560)

```
(SEQ ID NO: 44)
    1 maasrqivls qspailsasO gekvtltcra sssvsfmnwy qqcpgssOkp wiyatsnlas
  61 gvpgrfsgsg sgtsyslais rvqaqdaaty ycqqwnsnpl tfgagtklql kqlsggggsg
121 gggsggggsl qiqlvqsgpe lkkpgetvki sckasgytft nygmnwvkqa pgkglkwmgw
181 intytgqpty addfkgrfaf sletsaytay lqinnlkned matyfcarfs ygnsryadyw
241 gqgttltvss Og
```

This sequences should be identical to Russell's SEQ ID NO:6. It has three predicted Hyp, and no predicted glycosylated Hyp, based on the new standard method. However, based on other methods disclosed in this application, there are several predicted Hyp-glycosilation sites: Pro-48 ((excluded by the new standard method because of Lys-49), Pro-63, Pro-171 (excluded by new standard method because of Lys nearby), and Pro-251.
Russell also discloses L6 cys sFv, which differs from the above by the mutation K49C.
Anti-TAC sFV antibody, recognizes a portion of the IL2 receptor, (tobacco cells)
Sequence is shown in Russell's SEQ ID NO:8.

(SEQ ID NO: 119)

Met Ala Gln Val Gln Leu Gln Gln Ser Gly Ala Glu
Leu Ala Lys Pro Gly Ala Ser Val Lys Met Ser Cys
Lys Ala Ser Gly Tyr Thr Phe Thr Ser Tyr Arg Met
His Trp Val Lys Gln Arg Pro Gly Gln Gly Leu Glu
Trp Ile Gly Tyr Ile Asn Pro Ser Thr Gly Tyr Thr
Glu Tyr Asn Gln Lys Phe Lys Asp Lys Ala Thr Leu
Thr Ala Asp Lys Ser Ser Ser Thr Ala Tyr Met Gln
Leu Ser Ser Leu Thr Phe Glu Asp Ser Ala Val Tyr
Tyr Cys Ala Arg Gly Gly Gly Val Phe Asp Tyr Trp
Gly Gln Gly Thr Thr Leu Thr Val Ser Ser Gly Gly
Gly Gly Ser Gly Gly Gly Gly Ser Gly Gly Gly Gly
Ser Gln Ile Val Leu Thr Gln Ser Pro Ala Ile Met
Ser Ala Ser Pro Gly Glu Lys Val Thr Ile Thr Cys
Ser Ala Ser Ser Ser Ile Ser Tyr Met His Trp Phe
Gln Gln Lys Pro Gly Thr Ser Pro Lys Leu Trp Ile
Tyr Thr Thr Ser Asn Leu Ala Ser Gly Val Pro Ala
Arg Phe Ser Gly Ser Gly Ser Gly Thr Ser Tyr Ser
Leu Thr Ile Ser Arg Met Glu Ala Glu Asp Ala Ala
Thr Tyr Tyr Cys His Gln Arg Ser Thr Tyr Pro Leu
Thr Phe Gly Ser Gly Thr Lys Leu Glu Leu Lys

Our program implementing the new standard method predicts arabinogalactosylation of Pro 148 in the sequence SPG and arabinosylation of Pro 176 in the sequence SP. It predicts hydroxylation of Pro 191 in VPA it is likely a glycosylation site as well. It is unclear why the program doesn't arabinogalactosylate it as it fits the rules:
in the window:

### Sum of Hyp/Pro <4

### Sum of S/T/A/ >3 but <5

The number of different types of amino acids is >3 (it is 6)
The Hyp is not followed by a bulky residue.

### The sum of Y/K/H is not >1

According to our older prediction methods, Pro-141, Pro-148, Pro-176 and Pro-191 would be glycosylated Hyp, and there would also be an N-glycosylation site at positions 54-56.
Dragline silk protein [Nephila clavipes] (Tobacco plant, promoters, enhanced CaMV 35S promoter or tobacco cryptic constitutive promoter tCUP, Tobacco PR (Pathogenesis-Related)-S signal peptide, and ER retention signal (KDEL), MaSp1<0.0025% TSP, MaSp2 0.025%. Menassa et al., Plant Biotechnol. J. 2: 431-438

```
Spidroin 1 (MaSp1)
(SEQ ID NO: 46)
    1 aaaaaggagq ggygglgsqg agrggqgaga aaaaaggagq ggygglgsqg agrgglggqg
  61 agaaaaaaag gvgqgglggq gagqgagaaa aaaggagqgg ygglgsqgag rggsggqgag
121 aaaaaaggag qggygglgsq gagrgglggq gagaaaaaaa ggagqggygg lggqgagqgg
181 ygglgsqgag rgglggqgag aaaaaaagga gqgglggqga gqgagaaaaa aggagqggyg
241 glgsqgagrg gqgagaaaaa avgagqggyg gqgagqggyg glgsqgagrg glggqgagaa
301 aaaaaggagq gglggqgagq gagaaaaaag gagqggyggl gnqgagrggq gaaaaaagga
361 gqggygglgs qgagrgglgg qgagaaaaaa ggagqggygg lggqgagqgg ygglgsqgsg
421 rgglggqgag aaaaaaggag qgglggqgag qgagaaaaaa ggvrqggygg lgsqgagrgg
481 qgagaaaaaa ggagqggygg lggqgvgrgg lggqgagaaa aggagqggyg gvgsgasaas
541 aaasrlss#q assrvssavs nlvasgptns aalsstisnv vsqigasnpg lsgcdvliqa
601 llevvsaliq ilgsssi
```

One predicted AraGal-Hyp.

```
Spidroin 2 (MaSp2)
(SEQ ID NO: 48)
    1 pggygpgqqg pggygpgqqg psg#gsaaaa aaaaaagpgg ygpgqqgpgg ygpgqqgpgr
  61 ygpgqqgpsg #gsaaaaaag sgqqgpggyg prqqgpggyg qgqqgpsg#g saaaasaaas
121 aesgqqgpgg ygpgqqgpgg ygpgqqgpgg ygpgqqgpsg #gsaaaaaaa asgpgqqgpg
181 gygpgqqgpg gygpgqqgps g#gsaaaaaa aasgpgqqgp ggygpgqqgp ggygpgqqgl
241 sg#gsaaaaa aagpgqqgpg gygpgqqgps g#gsaaaaaa aaagpggygp gqqgpggygp
301 gqqgpsgags aaaaaaagpg qqglggygpg qqgpggygpg gqgpggyg#g sasaaaaaag
361 pgqqgpggyg pgqqgpsg#g sasaaaaaaa agpggygpgq qgpggyaOgq qgpsg#gsas
421 aaaaaaaagp ggygpgqqgp ggyaOgqqgp sg#gsaaaaa aaaagpggyg Oaqqgpsgpg
481 iaasaasagp ggygOaqqgp agyg#gsava asagagsagy g#gsqasaaa srlas#dsga
541 rvasavsnlv ssgptssaal ssvisnavsq igasnpglsg cdvliqalle ivsacvtils
601 sssigqvnyg aasqfaqvvg qsvlsaf
```

Many predicted AraGal-Hyp.

TABLE Q
Summary of information from Table P. All proteins
are human unless otherwise specified.

| | | | Pred |
|---|---|---|---|
| | | | Glyco |
| Protein | SEQ ID | Cells Expressed | Hyp |
| Green Fluorescent Protein | 70 | tobacco | N |
| Serum Albumin | 71 | tobacco | N |
| a1-antitrypsin | 72 | rice | N |
| bryodin 1 | 73 | tobacco | N |
| Hepatitis B Surface antigen | 74 | tobacco, potato | Y |
| monoclonal antibody versus | 75 | tobacco | N |
| Hepatitis B Surface | | | |
| antigen, heavy chain | | | |
| monoclonal antibody versus | 76 | tobacco | N |
| Hepatitis B Surface | | | |
| antigen, light chain | | | |
| interleukin-12, 35 kDa | 77 | tobacco | Y |
| interleukin-12, 40 kDa | 78 | tobacco | N |
| Single Chain Fv versus | 79 | tobacco | N |
| HBsAg | | | |
| Carrot Invertase | 80 | tobacco | N |
| erythropoietin | 22, 81 | tobacco | Y |
| lactoferrin | 82 | tobacco | Y |
| hirudin | 83 | Arabidopsis | N |
| milk beta casein | 84 | potato | Y |
| milk CD14 | 85 | tobacco | Y |
| GM-CSF | 86 | rice, tomato, | Y |
| | | tobacco | |
| Hemoglobin, alpha chain | 87 | tobacco | Y but see |
| | | | comment |
| Hemoglobin, beta chain | 88 | tobacco | Y but see |
| | | | comment |
| epidermal growth factor | 89 | tobacco | Y |
| protein C | 90 | tobacco | Y |
| growth hormone 1 | 33, 91 | tobacco | Y but see |
| | | | comment |
| interferon alpha-2b | 40, 92 | tobacco, potato | N |
| interferon beta | 93 | tobacco | N |
| placental alkaline | 94 | tobacco | Y |
| phosphatase | | | |
| interleukin-2 | 25, 95 | tobacco | Y |
| interleukin-4 | 96 | tobacco | N |
| muscarinic cholinergic | 97 | tobacco | Y |
| receptor m1 | | | |
| muscarinic cholinergic | 98 | tobacco | Y |
| receptor, m2 | | | |
| insulin-like growth factor | 39, 99 | tobacco | N |
| avidin | 100 | corn | N |
| human collagen alpha1 type I | 101 | tobacco | Y |
| bovine collagen alpha1 type | n/a | tobacco | ? |
| I, see Merle et al. | | | |
| phytase | 102 | tobacco | Y |
| xylanase | 103 | tobacco | Y |
| beta-glucoronidase | 104 | tobacco | Y |
| aprotonin | 105 | maize | N |
| heat-labile enterotoxin B | 106 | potato | N |
| subunit | | | |
| Norwalk virus capsid | 107 | tobacco, potato | Y |
| chymosin | 108 | tobacco, potato | Y |
| cholera toxin B subunit | 109 | tobacco, tomato | N |
| rabies virus glycoprotein | 110 | tomato | Y |
| foot and mouth disease | 111 | alfalfa | Y, but no |
| virus VP1 | | | signal |
| | | | peptide! |
| gastroenteritis coronavirus | 112 | Arabidopsis | Y |
| glycoprotein S | | | |
| avian reovirus sigma C | 113 | alfalfa | Y, but see |
| | | | comment |
| HIV-1 p24 | 114 | tobacco | Y |
| HIV-1 p24 fused to human | n/a | tobacco | Y |
| IgA | | | |
| Antibody versus | 115 | maize | Y |
| Glycoprotein D of herpes | | | |
| simplex virus, Human IgA1 | | | |
| heavy chain (sequence | | | |
| given is of hinge region) | | | |
| anti-rabies virus | 116 | tobacco | N |
| monoclonal antibody, heavy | | | |
| chain | | | |
| anti-rabies virus | 117 | tobacco | Y |
| monoclonal antibody, light chain | | | |
| Endo-1,4-beta-D-glucanase | 10 | tobacco, | Y |
| | | Arabidopsis | |
| Chimeric L6 antibody L6 sFv | 44 | tobacco | N, but see |
| (Russell's SEQ ID NO: 6) | | | comment |
| Chimeric L6 antibody L6 cys | — | tobacco | N, but see |
| sFv, which differs from the | | | comment |
| above by the mutation K49C. | | | |
| anti-TAC sFv antibody | 119 | tobacco | see |
| (Russell's SEQ ID NO: 8) | | | comment |
| Dragline silk protein | 46 | tobacco | Y |
| [Nephila clavipes, | | | |
| spidroin 1 | | | |
| Dragline silk protein | 48 | tobacco | Y |
| [Nephila clavipes], | | | |
| spidroin 2 | | | |

Citation of documents herein is not intended as an admission that any of the documents cited herein is pertinent prior art, or an admission that the cited documents is considered material to the patentability of any of the claims of the present application. All statements as to the date or representation as to the contents of these documents is based on the information available to the applicant and does not constitute any admission as to the correctness of the dates or contents of these documents.

The appended claims are to be treated as a non-limiting recitation of preferred embodiments.

In addition to those set forth elsewhere, the following references are hereby incorporated by reference, in their most recent editions as of the time of filing of this application: Kay, Phage Display of Peptides and Proteins: A Laboratory Manual; the John Wiley and Sons Current Protocols series, including Ausubel, Current Protocols in Molecular Biology; Coligan, Current Protocols in Protein Science; Coligan, Current Protocols in Immunology; Current Protocols in Human Genetics; Current Protocols in Cytometry; Current Protocols in Pharmacology; Current Protocols in Neuroscience; Current Protocols in Cell Biology; Current Protocols in Toxicology; Current Protocols in Field Analytical Chemistry; Current Protocols in Nucleic Acid Chemistry; and Current Protocols in Human Genetics; and the following Cold Spring Harbor Laboratory publications: Sambrook, Molecular Cloning: A Laboratory Manual; Harlow, Antibodies: A Laboratory Manual; Manipulating the Mouse Embryo: A Laboratory Manual; Methods in Yeast Genetics: A Cold Spring Harbor Laboratory Course Manual; Drosophila Protocols; Imaging Neurons: A Laboratory Manual; Early Development of Xenopus laevis: A Laboratory Manual; Using Antibodies: A Laboratory Manual; At the Bench: A Laboratory Navigator; Cells: A Laboratory Manual; Methods in Yeast Genetics: A Laboratory Course Manual; Discovering Neurons The Experimental Basis of Neuroscience; Genome Analysis: A Laboratory Manual Series; Laboratory DNA Science; Strategies for Protein Purification and Characterization: A Laboratory Course Manual; Genetic Analysis of Pathogenic Bacteria: A Laboratory Manual; PCR Primer: A Laboratory Manual; Methods in Plant Molecular Biology: A Laboratory Course Manual; Manipulating the Mouse Embryo: A Laboratory Manual; Molecular Probes of the Nervous System; Experiments with Fission Yeast: A Laboratory Course Manual; A Short Course in Bacterial Genetics: A Laboratory Manual and Handbook for Escherichia coli and Related Bacteria; DNA Science: A First Course in Recombinant DNA Technology; Methods in Yeast Genetics: A Laboratory Course Manual; Molecular Biology of Plants: A Laboratory Course Manual.

We also incorporate by reference the large number of sequence analysis tools listed on the www DOT expasy.org/tools/webpage (DOT used to disable hyperlink).

All references cited herein, including journal articles or abstracts, published, corresponding, prior or otherwise related U.S. or foreign patent applications, issued U.S. or foreign patents, or any other references, are entirely incorporated by reference herein, including all data, tables, figures, and text presented in the cited references. Additionally, the entire contents of the references cited within the references cited herein are also entirely incorporated by reference.

Reference to known method steps, conventional methods steps, known methods or conventional methods is not in any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

Any description of a class or range as being useful or preferred in the practice of the invention shall be deemed a description of any subclass (e.g., a disclosed class with one or more disclosed members omitted) or subrange contained therein, as well as a separate description of each individual member or value in said class or range.

The description of preferred embodiments individually shall be deemed a description of any possible combination of such preferred embodiments, except for combinations which are impossible (e.g., mutually exclusive choices for an element of the invention) or which are expressly excluded by this specification.

If an embodiment of this invention is disclosed in the prior art, the description of the invention shall be deemed to include the invention as herein disclosed with such embodiment excised.

### REFERENCE LIST H

The following references were sources for sequences used in designing the algorithm used to predict proline hydroxylation and Hyp-glycosylation, and are incorporated by reference in their entirety.
1. Goodrum, L. J., Patel, A., Leykam, J. F., and Kieliszewski, M. J. (2000) Phytochem. 54, 99-106
2. Schultz, C. J., Ferguson, K. L., Lahnstein, J., and Bacic, A. (2004) J. Biol. Chem. 279, 1-48
3. Du, H., Simpson, R. J., Moritz, R. L., Clarke, A. E., and Bacic, A. (1994) Plant Cell 6, 1643-1653
4. Shpak, E., Barbar, E., Leykam, J. F., and Kieliszewski, M. J. (2001) J. Biol. Chem. 276, 11272-11278
5. Shpak, E., Leykam, J. F., and Kieliszewski, M. J. (1999) Proc. Natl. Acad. Sci. U.S.A. 96, 14736-14741
6. Tan, L., Leykam, J., and Kieliszewski, M. J. (2003) Plant Physiol. 132, 1362-1369
7. Shpak, Elena. Synthetic genes for the elucidation of hydroxyproline O-glycosylation codes. 179. 2000. University of Ohio.

### Ref Type: Thesis/Dissertation

8. Zhao, Z. D., Tan, L., Showalter, A. M., Lamport, D. T. A., and Kieliszewski, M. J. (2002) Plant J. 31, 431-444
9. Gao, M., Kieliszewski, M. J., Lamport, D. T. A., and Showalter, A. M. (1999) Plant J. 18, 43-55
10. Chen, C.-G., Pu, Z.-Y., Moritz, R. L., Simpson, R. J., Bacic, A., Clarke, A. E., and Mau, S.-L. (1994) Proc. Natl. Acad. Sci. 91, 10305-10309
11. Motose, H., Sugiyama, M., and Fukuda, H. (2004) Nature 429, 873-878
12. Lindstrom, J. T. and Vodkin, L. O. (1991) Plant Cell 3, 561-571
13. Hong, J. C., Nagao, R. T., and Key, J. L. (1987) J. Biol. Chem. 262, 8367-8376
14. Frueauf, J. B., Dolata, M., Leykam, J. F., Lloyd, E. A., Gonzales, M., VandenBosch, K., and Kieliszewski, M. J. (2000) Phytochem. 55, 429-438
15. Wilson, R. C., Long, F., Maruoka, E. M., and Cooper, J. B. (1994) Plant Cell 6, 1265-1275
16. Mann, K., Schafer, W., Thoenes, U., Messerschmidt, A., Mahrabian, Z., and Nalbandyan, R. (1992) FEBS Lett. 314, 220-223
17. van Driessche, G., Dennison, C., Sykes, A. G., and Van Beeumen, J. (1995) Protein Science 4, 209-227
18. Esquerre-Tugaye, M. T. and Lamport, D. T. A. (1979) Plant Physiol. 64, 314-319
19. Smith, J. J., Muldoon, E. P., Willard, J. J., and Lamport, D. T. A. (1986) Phytochem. 25, 1021-1030
20. Lamport, D. T. A. (1969) Biochemistry 8, 1155-1163
21. Pearce, G. and Ryan, C. A. (2003) Journal of Biological Chemistry 278, 30044-30050
22. Osiecka, B. I., Ziolkowski, P., Gamian, E., Lis-Nawara, A., Marszalik, P., White, S. G., and Bonnett, R. (2003) Polish Journal of Pathology 54, 117-121
23. Sticher, L., Hofsteenge, J., Milani, A., Neubaus, J.-M., and Meins, F. (1992) Science 257, 655-657
24. Kieliszewski, M. J., Showalter, A. M., and Leykam, J. F. (1994) Plant J. 5, 849-861
25. Van Damme, E. J. M., Barre, A., Rouge, P., and Peumans, W. J. (2004) Plant Journal 37, 34-45
26. Li, X.-B., Kieliszewski, M. J., and Lamport, D. T. A. (1990) Plant Physiol. 92, 327-333
27. Fong, C., Kieliszewski, M. J., de Zacks, R., Leykam, J. F., and Lamport, D. T. A. (1992) Plant Physiol. 99, 548-552
28. Kieliszewski, M. J., O'Neill, M., Leykam, J., and Orlando, R. (1995) J. Biol. Chem. 270, 2541-2549
29. Kieliszewski, M. J., Kamyab, A., Leykam, J. F., and Lamport, D. T. A. (1992) Plant Physiol. 99, 538-547
30. Kieliszewski, M. J., Leykam, J. F., and Lamport, D. T. A. (1990) Plant Physiol. 92, 316-326
31. Stiefel, V., Perez-Grau, L., Albericio, F., Giralt, E., Ruiz-Avila, L., Ludevid, M. D., and Puigdomenech, P. (1988) Plant Mol. Biol. 11, 483-493
32. Li, L. C., Bedinger, P. A., Volk, C., Jones, A. D., and Cosgrove, D. J. (2003) Plant Physiology 132, 2073-2085

We claim:

1. A non-naturally occurring protein which is a mutant of a parental protein, differing from said parental protein at least in that, if both the mutant protein and the parental protein are expressed and secreted in plant cells, the mutant protein has a greater number of actual Hyp-glycosylation sites and/or a greater number of predictable Hyp-glycosylation sites than does the parental protein,
and which protein is not any of the following:
(a) (Ser-Hyp)32-EGFP, a fusion of (Ser-Hyp)32, SEQ ID NO: 65, to enhanced green fluorescent protein, or (GAGP)3-EGFP, a fusion of (GAGP)3, SEQ ID NO:66, to enhanced green fluorescent protein.,
(b) fusions of (SPP)24 (SEQ ID NO:67), (SPPP)15 (SEQ ID NO:68) or (SPPPP)18 (SEQ ID NO:69) to enhanced green fluorescent protein,
(c) mutants of sweet potato sporamin selected from the group consisting of the deletion mutants delta23-26, delta27-30, delta31-34, and, in the delta25-30 background, single substitution mutants in which one of residues 31-35 or 37-41 was replaced with another amino acid, or
(d) a protein listed in Table Q whose name is italicized in that table.

2. The protein of claim 1 for which Hyp-glycosylation sites were predicted by the new standard method.

3. The protein of claim 2 for which Pro-hydroxylation sites were predicted by the standard qualitative method.

4. The protein of claim 2 for which Pro-hydroxylation sites were predicted by the quantitative standard method, using the default parameters.

5. The protein of claim 4 which is a mutant of a parental protein, differing from said parental protein at least in that
(A) it comprises at least one proline which has a higher Hyp-score than that of an aligned proline in the parental protein, and/or
(B) it comprises at least one proline, with a Hyp-score, given the default value (0.4) for the local composition factor baseline, which is greater than 0.5, for which the aligned amino acid, if any, in the parental protein is not a proline,
and which
(I) comprises a sequence which is at least 50% identical, according to the primary or secondary definition of percentage identity, to the amino acid sequence of said parental protein, and which protein either substantially retains at least one biological activity (other than an immunological activity) of said parental protein, or
(II) is specifically cleavable to release a second protein which comprises a sequence which is at least 50% identical, according to the primary or secondary definition of percentage identity, to the amino acid sequence of said parental protein and substantially retains at least one biological activity (other than an immunological activity) of said parental protein.

6. The protein of any one of the preceding claims in which the parental protein is a non-plant protein.

7. The protein of claim 6 in which the parental protein is a vertebrate protein.

8. The protein of claim 6 in which the parental protein is a mammalian protein.

9. The protein of claim 6 in which the parental protein is a human protein.

10. The protein of any one of claims 1-5 in which the parental protein is a plant protein which is not naturally secreted by plant cells.

11. The protein of any one of claims 1-5 in which the parental protein is a protein which does not possess any Hyp-glycosylation sites.

12. The protein of any one of claims 1-11 wherein the mature portion of the translated sequence of the secreted protein is at least 95% identical, according to the primary definition of percentage identity, to the mature portion of the translated sequence of the parental protein.

13. The protein of any one of claims 1-12, wherein the protein comprises at least one N-glycosylation site which does not occur in the parental protein.

14. The protein of claim 13, wherein the presence of said N-glycosylation site results in increased secretion in a suitable plant cell.

15. In a method of producing a protein, the improvement comprising expressing and secreting a protein according to any one of claims 1-14 in plant cells, wherein one or more of the prolines are hydroxylated, and one or more of the resulting hydroxyprolines is glycosylated.

16. In a method of producing a protein, comprising expressing and secreting a protein in a plant cell, the improvement comprising said protein being one which is not secreted by plant cells in nature, and which, when expressed in said plant cells, undergoes proline-hydroxylation and Hyp-glycosylation,
with the following exceptions:
(I) the expression and secretion, in tobacco cells, of
(a) (Ser-Hyp)32-EGFP, a fusion of (Ser-Hyp)32, SEQ ID NO: 65, to enhanced green fluorescent protein, or (GAGP)3-EGFP, a fusion of (GAGP)3, SEQ ID NO:66, to enhanced green fluorescent protein.,
(b) fusions of (SPP)24 (SEQ ID NO: 67), (SPPP)15 (SEQ ID NO:68) or (SPPPP)18 (SEQ ID NO:69) to enhanced green fluorescent protein,
(c) mutants of sweet potato sporamin selected from the group consisting of the deletion mutants, delta23-26, delta27-30, delta31-34, and, in the delta25-30 background, single substitution mutants in which one of residues 31-35 or 37-41 was replaced with another amino acid, and
(II) the expression and secretion of the mature form of one of the proteins set forth in column 1 of Table Q, in plant cells of the kind specified, for that protein, in column 3 of table Q, with the exception of foot and mouth disease virus VP1.

17. The method of claim 16 in which the protein is a one predisposed to Hyp-glycosylation.

18. The protein or method of any one of claims 1-17 wherein the secreted protein comprises at least two predicted and/or actual Hyp glycosylation sites.

19. The protein or method of any one of claims 1-18 wherein the secreted protein is not a disulfide bonded protein.

20. The protein or method of any one of claims 1-19 wherein the secreted protein comprises at least one substitution, deletion or internal insertion Hyp-glycomodule.

21. The protein or method of claim 20 wherein the secreted protein comprises at least one substitution Hyp-glycomodule.

22. The protein or method of any one of claims 1-21 wherein the secreted protein comprises at least one native Hyp-glycomodule.

23. The protein or method of any one of claims 20-22 wherein the secreted protein further comprises at least addition Hyp-glycomodule.

24. The protein or method of any one of claims 1-23, wherein the protein comprises at least one large Hyp block.

25. The protein or method of any one of claims 1-24, wherein the protein comprises at least one dipeptidyl Hyp block.

26. The protein or method of any one of claims 1-25, wherein the protein comprises at least one cluster of non-contiguous Hyp residues.

27. The protein or method of any one of claims 1-26, wherein the protein comprises at least one isolated Hyp residue.

28. The protein or method of any one of claims 1-27, wherein the protein comprises at least one arabinosylated Hyp residue.

29. The protein or method of any one of claims 1-28, wherein the protein comprises at least one arabinogalactosylated Hyp residue.

30. The method of any one of claims 15-29 wherein the level of secretion of the protein is at least 1% total secreted protein.

31. The protein of claim 1 which comprises at least one substitution Hyp-glycomodule.

32. The method of claim 15 wherein the mutant protein comprises at least one substitution Hyp-glycomodule.

33. The method of claim 32 wherein the level of secretion of the protein is at least 1% total secreted protein.

34. The method of claim 32 wherein the level of secretion of the protein is at least ten-fold greater than the level of secretion of the parental protein wider the same conditions, such conditions comprising the same signal peptide, the same promoter, and the same strain of plant cell.

35. The protein of claim 1 for which Hyp-glycosylation sites were predicted by the old standard method.

36. The method of claim 15 for which Hyp-glycosylation was predicted by the new standard method.

37. The method of claim 15 for which Hyp-glycosylation was predicted by the old standard method.

38. The method of claim 15, 36 or 37 for which Pro-hydroxylation was predicted by the standard quantitative method.
